(19) United States
(12) Patent Application Publication
(10) Pub. No.: US 2022/0227888 A1
(43) Pub. Date: Jul. 21, 2022

(54) BISPECIFIC BINDING CONSTRUCTS WITH SELECTIVELY CLEAVABLE LINKERS

(71) Applicant: AMGEN INC., Thousand Oaks, CA (US)

(72) Inventors: Johannes BROZY, Munich (DE); Pavan GHATTYVENKATAKRISHNA, Cambridge, MA (US); Brendan AMER, Los Angeles, CA (US)

(73) Assignee: AMGEN INC., Thousand Oaks, CA (US)

(21) Appl. No.: 17/616,575

(22) PCT Filed: Jun. 5, 2020
(86) PCT No.: PCT/US2020/036474

§ 371 Date: Dec. 3, 2021

Related U.S. Application Data
(60) Provisional application No. 62/858,509, filed on Jun. 7, 2019.
(60) Provisional application No. 62/858,630, filed on Jun. 7, 2019.

(51) Int. Cl. C07K 16/46; C07K 14/725; C07K 16/28; A61K 39/395; A61K 45/06; C07K 14/765

(52) U.S. Cl. CPC C07K 16/468; C07K 14/7051; C07K 16/2809; A61K 39/3955; A61K 45/06; C07K 2317/56; C07K 2317/31; C07K 2317/66; C07K 2317/622; C07K 2317/64; C07K 14/765

(57) ABSTRACT

New formats of bispecific binding constructs with protease cleavable linkers are described, as well as their methods of making. Additionally, uses in therapeutic indications are also described.

### CROSS-REFERENCE TO RELATED APPLICATIONS

This application claims priority to U.S. Provisional Application No. 62/858,509, filed Jun. 7, 2019 and U.S. Provisional Application No. 62/858,630, filed Jun. 7, 2019. The above-identified applications are each hereby incorporated herein by reference for all purposes.

### REFERENCE TO THE SEQUENCE LISTING

The instant application contains a Sequence Listing which has been submitted electronically in ASCII format and is hereby incorporated by reference in its entirety. Said ASCII copy, created on Jun. 4, 2020, is named A-2406-WO-PCT_SL.txt and is 164,621 bytes in size.

### FIELD

The invention is in the field of protein engineering.

### BACKGROUND

Bispecific binding constructs have shown therapeutic promise in recent years. For example, a bispecific binding construct that targets both CD3 and CD19 in a bispecific T cell Engager (BiTE®) format has shown impressive efficacy at low doses. Bargou et al. (2008), Science 321: 974-978. This BiTE® format comprises two scFv's, one of which targets CD3 and one of which targets a tumor antigen, CD19, joined by a flexible linker. This unique design allows the bispecific binding construct to bring activated T-cells into proximity with target cells, resulting in cytolytic killing of the target cells. See, for example, WO 99/54440A1 (U.S. Pat. No. 7,112,324 B1) and WO 2005/040220 (U.S. Patent Appl. Publ. No. 2013/0224205A1). Later developments were bispecific binding constructs binding to a context independent epitope at the N-terminus of the CD3ϵ chain (see WO 2008/119567; U.S. Patent Appl. Publ. No. 2016/0152707A1).

In the biopharmaceutical industry, molecules can exhibit undesirable, detrimental side effects in patients receiving treatment, particularly where the drug is active upon administration to the patient. In small molecule pharmaceuticals, for example, these side effects can be minimized by administering inactive prodrugs that become active once metabolized. Bispecific binding constructs that mediate cellular cytotoxicity can exhibit some of these undesirable side effects. Accordingly, there is a need in the art for bispecific therapeutics with favorable pharmacokinetic properties, as well as therapeutic efficacy, and a format that provides efficient production, increased stability, and minimized side effects.

### SUMMARY

Described herein are several new formats of bispecific binding constructs. In one embodiment, the invention provides a bispecific binding construct comprising a polypeptide chain comprising an amino acid sequence having the formula VH1-L1-VH2-L2-VL1-L3-VL2, wherein VH1 and VH2 comprise immunoglobulin heavy chain variable regions, VL1 and VL2 comprise immunoglobulin light chain variable regions, and L1, L2 and L3 are linkers, wherein L1 is at least 10 amino acids, L2 is at least 15 amino acids and L3 is at least 10 amino acids, wherein L1 or L3 comprises a protease cleavage site, and wherein the bispecific binding construct can bind to an immune effector cell and a target cell.

In another embodiment, the invention provides a bispecific binding construct comprising a polypeptide chain comprising an amino acid sequence having the formula VH1-L1-Fc-L2-VH2-L3-VL1-L4-Fc-L5-VL2, wherein VH1 and VH2 comprise immunoglobulin heavy chain variable regions, VL1 and VL2 comprise immunoglobulin light chain variable regions, Fc comprises an immunoglobulin heavy chain constant domain-2 and an immunoglobulin heavy chain constant domain-3, and L1, L2, L3, L4, and L5 are linkers, wherein L1 is at least 10 amino acids, L2 is at least 10 amino acids, L3 is at least 15 amino acids, L4 is at least 10 amino acids, and L5 is at least 10 amino acids, and wherein L1, L2, L4 and L5 further comprise a protease cleavage site of at least 5 amino acids, and wherein the bispecific binding construct can bind to an immune effector cell and a target cell.

In further embodiments, the invention provides a nucleic acid encoding the bispecific binding constructs described herein, and vectors comprising these nucleic acids. Further, the invention provides a host cell comprising the vectors described herein.

In yet other embodiments, the invention provides a method of manufacturing the bispecific binding constructs described herein comprising (1) culturing a host cell under conditions to express the bispecific binding construct and (2) recovering the bispecific binding construct from the cell mass or cell culture supernatant, wherein the host cell comprises one or more nucleic acid(s) encoding any of the bispecific binding constructs described herein.

In other embodiments, the invention provides a method of treating a cancer patient comprising administering to the patient a therapeutically effective amount of the bispecific binding constructs described herein.

In other embodiments, the invention provides a method of treating a patient having an infectious disease comprising administering to the patient a therapeutically effective amount of the bispecific binding constructs described herein.

In other embodiments, the invention provides a method of treating a patient having an autoimmune, inflammatory, or fibrotic condition comprising administering to the patient a therapeutically effective amount of the bispecific binding constructs described herein.

In another embodiment, the invention provides a pharmaceutical composition comprising the bispecific binding constructs described herein.

### BRIEF DESCRIPTION OF DRAWINGS

FIG. 1. A representative diagram of an exemplary embodiment of a HHLL formats A and B, and indicating where protease cleavage sites, cysteine clamps, and the optional CD3ϵ (for formats A and B) and scFc moieties (for format A) are located.

FIG. 2. A representative diagram of an exemplary embodiment of a HHLL formats C and D, and indicating where protease cleavage sites, cysteine clamps, and the optional CD3ϵ (for format C), the optional HSA-CD3_{a.a 1-6 or 1-27} (for format D), and scFc moieties (for formats C and D) are located.

FIG. 3. A representative diagram of an exemplary embodiment of a HHLL format E indicating where protease cleavage sites, cysteine clamps, and the optional scFc-CD3ϵ moiety is located.

FIG. 4. A chromatography readout indicating proper expression of bispecific construct N4J.

FIG. 5. A chromatography readout, and SDS-PAGE indicating proper expression of bispecific construct N7A.

FIG. 6. A chromatography readout, and SDS-PAGE indicating expression of bispecific construct V1E, but with a lower molecular weight than expected.

FIG. 7. A chromatography readout, and SDS-PAGE indicating proper expression of bispecific construct B1U.

FIG. 8. A chromatography readout, and SDS-PAGE indicating proper expression of bispecific construct Z9P.

FIG. 9. A chromatography readout, and SDS-PAGE indicating proper expression of bispecific construct O7H.

FIG. 10. A chromatography readout, and SDS-PAGE indicating proper expression of bispecific construct W9A.

FIG. 11. A chromatography readout, and SDS-PAGE indicating proper expression of bispecific construct B2P.

FIG. 12. A chromatography readout, and SDS-PAGE indicating proper expression of bispecific construct T7U.

FIG. 13. A chromatography readout, and SDS-PAGE indicating proper expression of bispecific construct L2G.

FIG. 14A. SDS-PAGE of bispecific constructs (N4J, W2K, N7A, W9A and B2P) in presence or absence of recombinant human MMP-9.

FIG. 14B. SDS-PAGE of bispecific constructs (W2K, Z9P, V1E, B1U, T7U and L2G) in presence or absence of recombinant human MMP-9.

FIGS. 15A and 15B. FACS analysis of binding to CD3 expressing cells (FIG. 15A) and mesothelin expressing cells (FIG. 15B) by bispecific construct N4J with protease activation and without protease activation.

FIG. 16. FACS analysis of binding to CD3 and mesothelin positive cells by bispecific construct N7A with protease activation and without protease activation.

FIG. 17. FACS analysis of binding to CD3 and mesothelin positive cells by bispecific constructs W2K, V1E without protease activation, B1U, Z9P with protease activation and without protease activation.

FIG. 18. FACS analysis of binding to CD3 positive cells by bispecific constructs B2P, W9A and N7A with protease activation and without protease activation.

FIG. 19. FACS analysis of binding to mesothelin positive cells by bispecific constructs B2P, W9A and N7A with protease activation and without protease activation.

FIG. 20. FACS-based in vitro cytotoxicity assay of bispecific constructs N4J and W2K with protease activation and without protease activation.

FIG. 21. FACS-based in vitro cytotoxicity assay of bispecific constructs N7A, W2K and neg. control with protease activation and without protease activation.

FIG. 22. FACS-based in vitro cytotoxicity assay of bispecific constructs Z9P, V1E, B1U and neg. control with protease activation and without protease activation.

FIG. 23. FACS-based in vitro cytotoxicity assay of bispecific constructs W9A, B2P, N7A with protease activation and without protease activation.

FIG. 24. FACS-based in vitro cytotoxicity assay of bispecific constructs N7A, O7H and B2P with protease activation and without protease activation.

FIG. 25. FACS-based in vitro cytotoxicity assay of bispecific constructs T7U, L2G, N7A and B2P with protease activation and without protease activation.

FIG. 26. Overview of EC₅₀ spans, shift factor of EC₅₀ values and number of in vitro cytotoxicity assays performed for each bispecific construct with protease activation and without protease activation.

### DETAILED DESCRIPTION

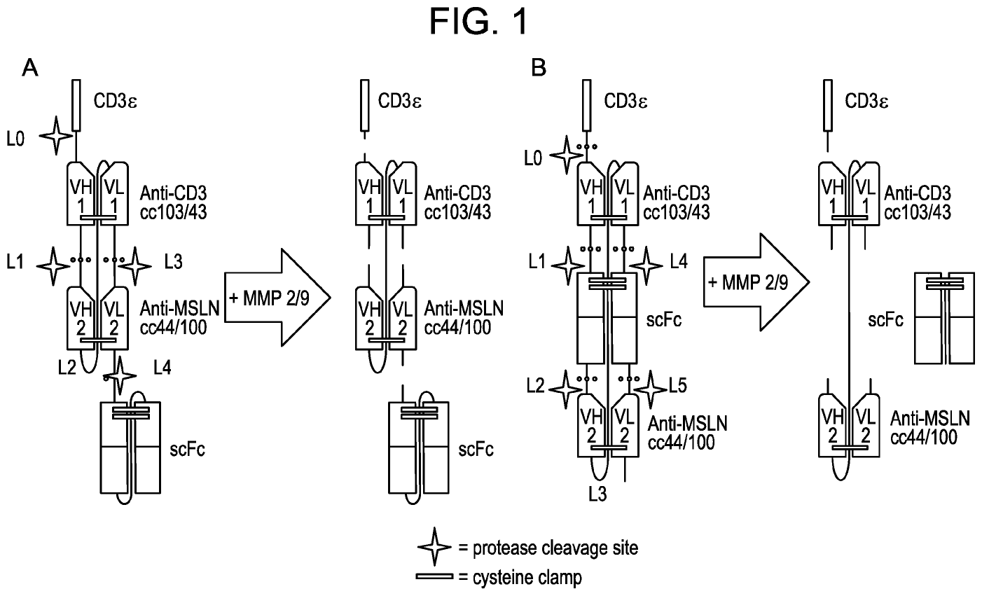

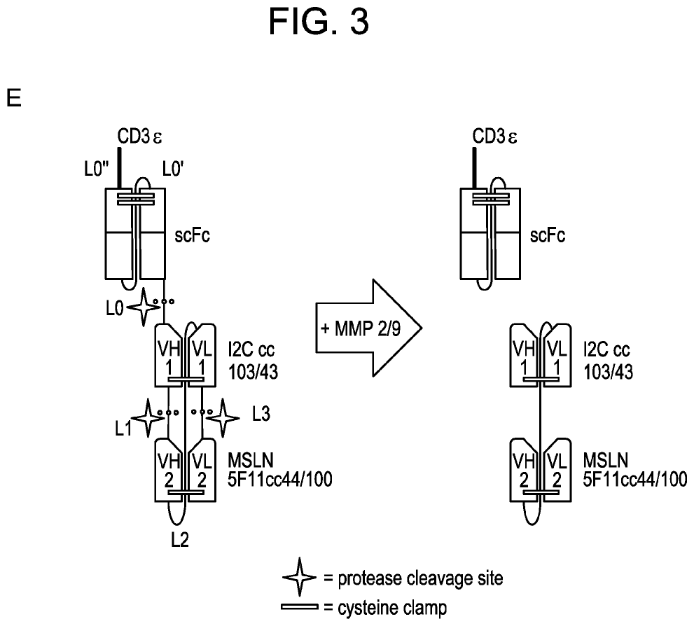

Described herein are novel formats for bispecific binding constructs. FIGS. 1-3 depict representative example formats (A-E) of these constructs. In one embodiment, this format comprises a single polypeptide chain that comprises two immunoglobulin variable heavy chain (VH) regions, two immunoglobulin variable light chain (VL) regions, a protease cleavage site, and optionally, and Fc region, arranged in the following order: VH1-VH2-VL1-VL2 (“HHLL”) and more specifically, in a first format VH1-linker-VH2-linker-VL1-linker-VL2, optionally with another linker after the VL2 and an scFc or other half-life extending moiety, and a second format VH1-linker-CH2-CH3-linker-VH2-linker-VL1-CH2-CH3-linker-VL2 . This bispecific construct HHLL format provides both enhanced stability and increased in vitro expression as compared to, for example, an HLHL format, yet it maintains the intended function of binding the desired targets on the immune effector cell and the target cell. Accordingly, the present HHLL format provides bispecific molecules that can be produced more efficiently and have greater stability, characteristics that are sought after in a pharmaceutical composition.

Specific numbered embodiments provided by the invention include, but are not limited to, the following:

1. A bispecific binding construct comprising a polypeptide chain comprising an amino acid sequence having the formula VH1-L1-VH2-L2-VL1-L3-VL2, wherein VH1 and VH2 comprise immunoglobulin heavy chain variable regions, VL1 and VL2 comprise immunoglobulin light chain variable regions, and L1, L2 and L3 are linkers, wherein L1 is at least 10 amino acids, L2 is at least 15 amino acids and L3 is at least 10 amino acids, wherein L1 or L3 comprises a protease cleavage site, and wherein the bispecific binding construct can bind to an immune effector cell and a target cell.

2. A bispecific binding construct comprising a polypeptide chain comprising an amino acid sequence having the formula VH1-L1-scFc_{Subdomain1}-L2-VH2-L3-VL1-L4-scFc_{Subdomain2}-L5-VL2, wherein VH1 and VH2 comprise immunoglobulin heavy chain variable regions, VL1 and VL2 comprise immunoglobulin light chain variable regions, scFc comprises subdomain 1 or subdomain 2 of an immunoglobulin heavy chain constant domain-2 and an immunoglobulin heavy chain constant domain-3, and L1, L2, L3, L4, and L5 are linkers, wherein L1 is at least 10 amino acids, L2 is at least 10 amino acids, L3 is at least 15 amino acids, L4 is at least 10 amino acids, and L5 is at least 10 amino acids, and wherein L1, L2, L4 and L5 further comprise a protease cleavage site of at least 5 amino acids, and wherein the bispecific binding construct can bind to an immune effector cell and a target cell.

3. The bispecific binding construct of embodiment 1, wherein the protease cleavage site is present in both L1 and L3.

4. The bispecific binding construct of embodiment 1 or 3, further comprising at least one cysteine clamp.

5. The bispecific binding construct of embodiment 4, wherein the cysteine clamp is located in a position to facilitate linkage between the VH1 and VL1 subunits, the VH2 and VL2 subunits, or the scFc subunits.

6. The bispecific binding construct of embodiment 2, further comprising at least one cysteine clamp.

7. The bispecific binding construct of embodiment 6, wherein the cysteine clamp is located in a position to facilitate linkage between the VH1 and VL1 subunits, the VH2 and VL2 subunits, and/or the scFc subunits.

8. The bispecific binding construct of any of embodiments 1-7, further comprising a half-life extending moiety linked to the VL2 domain.

9. The bispecific binding construct of embodiment 8, wherein the half-life extending moiety comprises an additional linker and a single chain immunoglobulin Fc region (scFc) encoding a human IgG1, IgG2, or IgG4 antibody.

10. The bispecific binding construct of embodiment 9, wherein the additional linker comprises a protease cleavage site.

11. The bispecific binding construct of embodiment 10, wherein the scFc polypeptide chain comprises one or more alterations that inhibit Fc gamma receptor (FcγR) binding and/or one or more alterations that extends half-life.

12. The bispecific binding construct of any of embodiments 1-11, wherein the VH1, VH2, VL1, and VL2 all have different sequences.

13. The bispecific binding construct of any of embodiments 1-12, wherein
a. the VH1 sequence comprises SEQ ID NO: 65 or 67, and the VL1 sequence comprises SEQ ID NO: 66 or 68, and the VH2 sequence comprises SEQ ID NO: 75 or 77, and the VL2 sequence comprises SEQ ID NO: 76 or 78, or
b. the VH1 sequence comprises SEQ ID NO: 75 or 77, and the VL1 sequence comprises SEQ ID NO: 76 or 78, and the VH2 sequence comprises SEQ ID NO: 65 or 67, and the VL2 sequence comprises SEQ ID NO: 66 or 68.

14. The bispecific binding construct of any of embodiments 1-13, further comprising an additional moiety linked to the VH1 with an additional linker (L0), wherein L0 is at least 5 amino acids in length.

15. The bispecific binding construct of embodiment 14, wherein the additional moiety is a CD3ϵ, or a human serum albumin-linker-CD3(a.a. 1-6) or a human serum albumin-linker-CD3(a.a. 1-27), or an scFc-linker-CD3ϵ.

16. The bispecific binding construct of embodiment 14 or 15, wherein L0 further comprises a protease site.

17. The bispecific binding construct of any of embodiments 1-16, wherein the linkers are different lengths.

18. The bispecific binding construct of any of embodiments 1-16, wherein the linkers are the same length.

19. The bispecific binding construct of any of embodiments 1-16, wherein L1 and L2 are the same length.

20. The bispecific binding construct of any of embodiments 1-16, wherein L1 and L3 are the same length.

21. The bispecific binding construct of any of embodiments 1-16, wherein L2 and L3 are the same length.

22. The bispecific binding construct of any of embodiments 1-16, wherein the amino acid sequence of L1 is at least 10 amino acids long, the amino acid sequence of L2 is at least 15 amino acids long, and the amino acid sequence of L3 is at least 15 amino acids long.

23. The bispecific binding construct of any of embodiments 1-22, wherein the effector cell expresses an effector cell protein that is part of a human T cell receptor (TCR)-CD3 complex.

24. The bispecific binding construct of any of embodiments 1-22, wherein the effector cell protein is the CD3ϵ chain

25. A nucleic acid encoding the bispecific binding construct of any of embodiments 1-24.

26. A vector comprising the nucleic acid of embodiment 25.

27. A host cell comprising the vector of embodiment 26.

28. A method of manufacturing the bispecific binding construct of any of embodiments 1-24 comprising (1) culturing a host cell under conditions so as to express the bispecific binding construct and (2) recovering the bispecific binding construct from the cell mass or cell culture supernatant, wherein the host cell comprises one or more nucleic acid(s) encoding bispecific binding construct of any of any of embodiments 1-24.

29. A method of treating a cancer patient comprising administering to the patient a therapeutically effective amount of the bispecific binding construct of any of embodiments 1-24.

30. The method of embodiment 29, wherein a chemotherapeutic agent, a non-chemotherapeutic anti-neoplastic agent, and/or radiation is administered to the patient concurrently with, before, or after administration of the bispecific binding construct.

31. A pharmaceutical composition comprising the bispecific binding construct of any of embodiments 1-24.

32. The use of the bispecific binding construct of any of embodiments 1-24 in the manufacture of a medicament for the prevention, treatment or amelioration of a disease.

33. The bispecific binding construct of any of embodiments 1-24, wherein the binding construct amino acid sequence comprises a sequence selected from SEQ ID NOs: 88, 89, 90, 91, 92, 93, 94, 95, 96, 97, or 98.

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references.

Polynucleotide and polypeptide sequences are indicated using standard one- or three-letter abbreviations. Unless otherwise indicated, polypeptide sequences have their amino termini at the left and their carboxy termini at the right, and single-stranded nucleic acid sequences, and the top strand of double-stranded nucleic acid sequences, have their 5′ termini at the left and their 3′ termini at the right. A particular section of a polypeptide can be designated by amino acid residue number such as amino acids 1 to 50, or by the actual residue at that site such as asparagine to proline. A particular polypeptide or polynucleotide sequence also can be described by explaining how it differs from a reference sequence.

### Definitions

The term “isolated” in reference to a molecule (where the molecule is, for example, a polypeptide, a polynucleotide, a bispecific binding construct, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In one embodiment, the nucleic acid molecules of the invention comprise a contiguous open reading frame encoding an antibody, or a fragment, derivative, mutein, or variant thereof, of the invention.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals).

A “host cell” is a cell that can be used to express a nucleic acid, e.g., a nucleic acid of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

A “single-chain variable fragment” (“scFv”) is a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., Science 242:423-26 (1988) and Huston et al., 1988, Proc. Natl. Acad. Sci. USA 85:5879-83 (1988)). When in the context of other additional moieties (e.g., an Fc region), the scFv can be arranged VH-linker-VL, or VL-linker-VH, for example.

The term “CDR” refers to the complementarity determining region (also termed “minimal recognition units” or “hypervariable region”) within antibody variable sequences. The CDRs permit the antibody or the bispecific binding construct to specifically bind to a particular antigen of interest and the bispecific binding constructs provided herein may comprise CDRs from the heavy chain and/or the light chain. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it a bispecific binding construct.

The term “human antibody” includes antibodies having antibody regions such as variable and constant regions or domains which correspond substantially to human germline immunoglobulin sequences known in the art, including, for example, those described by Kabat et al. (1991) (loc. cit.). The human antibodies referred to herein may include amino acid residues not encoded by human germline immunoglobulin sequences (e.g., mutations introduced by random or site-specific mutagenesis in vitro or by somatic mutation in vivo), for example in the CDRs, and in particular, in CDR3. The human antibodies can have at least one, two, three, four, five, or more positions replaced with an amino acid residue that is not encoded by the human germline immunoglobulin sequence. The definition of human antibodies as used herein also contemplates fully human antibodies, which include only non-artificially and/or genetically altered human sequences of antibodies as those can be derived by using technologies or systems known in the art, such as for example, phage display technology or transgenic mouse technology, including but not limited to the Xenomouse. In the context of the present invention, the variable regions from a human antibody can be used in the bispecific binding construct formats contemplated.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) hinge, CH2 and CH3 domains from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293. In the context of the present invention, the variable regions from a humanized antibody can be used in the bispecific binding construct formats contemplated.

The term “chimeric antibody” refers to an antibody that contains one or more regions from one antibody and one or more regions from one or more other antibodies. In one embodiment, one or more of the CDRs are derived from a human antibody. In another embodiment, all of the CDRs are derived from a human antibody. In another embodiment, the CDRs from more than one human antibodies are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also included are fragments of such antibodies that exhibit the desired biological activity. In the context of the present invention, the variable regions from a chimeric antibody can be used in the bispecific binding construct formats contemplated.

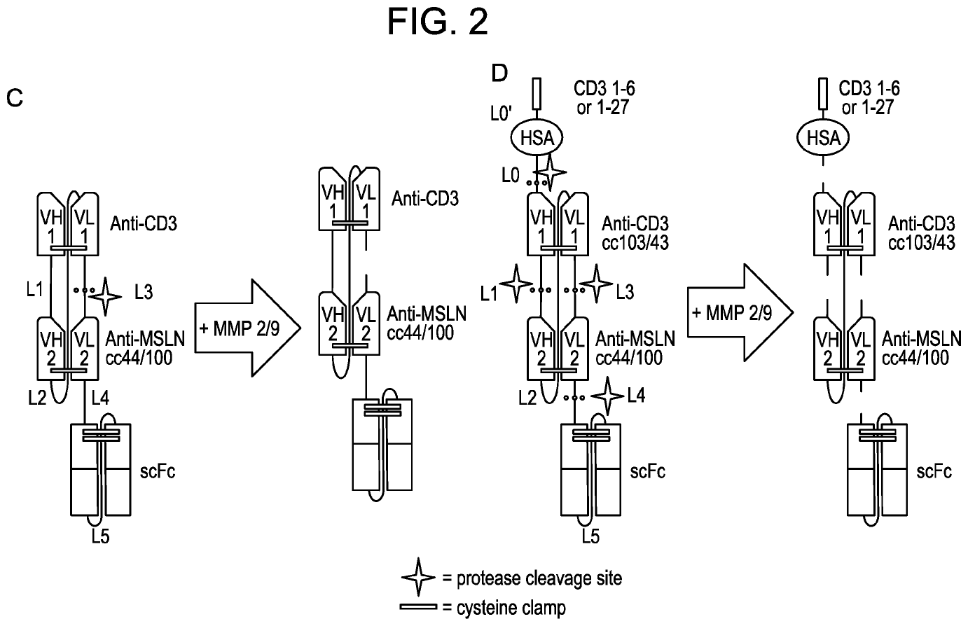

The invention provides bispecific binding constructs that comprise the HHLL format and further comprise linkers comprising protease cleavage sites. In the most general sense, a bispecific binding construct as described herein comprises several polypeptide chains having different amino acid sequences, which, when linked together, can bind to two different antigens. With the inclusion of a protease cleavage site in particular linkers (see, e.g., FIGS. 1 and 2), the binding construct in uncleaved form has reduced or no binding to a desired target. Once exposed to protease, the linkers are cleaved and the binding construct is then able to bind a desired target. Optionally, the HHLL molecules further comprise a half-life extending moiety. In some embodiments, the half-life extending moiety is an Fc polypeptide chain. In other embodiments, the half-life extending moiety is a single-chain Fc. In yet other embodiment, the half-life extending moiety is a hetero-Fc. In yet other embodiments, the half-life extending moiety is human albumin.

### Linkers

Between the immunoglobulin variable regions is a peptide linker, which can be the same linker or different linkers of different lengths. The linkers can play a role in the structure of the bispecific binding construct. If the linker is too short, it will not allow enough flexibility for the appropriate variable regions on a single polypeptide chain to interact to form an antigen binding site. If the linker is the appropriate length, it will allow a variable region to interact with another variable region on the same polypeptide chain to form an antigen binding site. In certain embodiments, the HHLL format comprises disulfide bonds—both intra-domain (within H1, L1) and inter-domain (between H1 and L1). In order to achieve proper expression and conformation of the bispecific binding constructs of the invention, in certain embodiments specific linkers are used between the various immunoglobulin regions (see, e.g., FIG. 1 herein). Exemplary linkers are provided in Table 1 herein. In certain embodiments, increasing linker length might result in increased protein clipping, an undesirable property. Accordingly, it is desirable to achieve the appropriate balance between linker length to allow proper polypeptide structure and activity, yet not result in increased clipping.

A “linker,” as meant herein, is a peptide that links two polypeptides. In certain embodiment, a linker can link two immunoglobulin variable regions in the context of a bispecific binding construct. A linker can be from 2-30 amino acids in length. In some embodiments, a linker can be 2-25, 2-20, or 3-18 amino acids long. In some embodiments, a linker can be a peptide no more than 14, 13, 12, 11, 10, 9, 8, 7, 6, or 5 amino acids long. In other embodiments, a linker can be 5-25, 5-15, 4-11, 10-20, or 20-30 amino acids long. In other embodiments, a linker can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long. Exemplary linkers include, for example, the amino acid sequences GGGGS (SEQ ID NO: 1), GGGGSGGGGS (SEQ ID NO: 2), GGGGSGGGGSGGGGS (SEQ ID NO: 3), GGGGSGGGGSGGGGSGGGGS (SEQ ID NO: 4), GGGGSGGGGSGGGGSGGGGSGGGGS (SEQ ID NO: 5), GGGGQ (SEQ ID NO: 6), GGGGQGGGGQ (SEQ ID NO: 7), GGGGQGGGGQGGGGQ (SEQ ID NO: 8), GGGGQGGGGQGGGGQGGGGQ (SEQ ID NO: 9), GGGGQGGGGQGGGGQGGGGQGGGGQ (SEQ ID NO: 10), GGGGSAAA (SEQ ID NO: 11), TVAAP (SEQ ID NO: 12), ASTKGP (SEQ ID NO: 13), and AAA (SEQ ID NO: 14), among others, including repeats of the aforementioned amino acid sequences or subunits of amino acid sequences (e.g., GGGGS (SEQ ID NO: 1) or GGGGQ (SEQ ID NO: 6) repeats).

In certain embodiments in the context of the HHLL molecules of the invention, the linker sequence of Linker 1 is at least 10 amino acids. In other embodiments, Linker 1 is at least 15 amino acids. In other embodiments, Linker 1 is at least 20 amino acids. In other embodiments, Linker 1 is at least 25 amino acids. In other embodiments, Linker 1 is at least 30 amino acids. In other embodiments, Linker 1 is 10-30 amino acids. In other embodiments, Linker 1 is 10, 11, 12, 13, 14, 15, 16, 17, 18, 19, 20, 21, 22, 23, 24, 25, 26, 27, 28, 29, or 30 amino acids. In yet other embodiments, Linker 1 is greater than 30 amino acids.

In certain embodiments in the context of the HHLL molecules of the invention, the linker sequence of Linker 2 is at least 15 amino acids. In other embodiments, Linker 2 is at least 20 amino acids. In other embodiments, Linker 2 is at least 25 amino acids. In other embodiments, Linker 2 is at least 30 amino acids. In other embodiments, Linker 2 is 15-30 amino acids. In other embodiments, Linker 2 is 15, 16, 17, 18, 19, 20, 21, 22, 23, 24, 25, 26, 27, 28, 29, or 30 amino acids. In yet other embodiments, Linker 2 is greater than 30 amino acids.

In certain embodiments in the context of the HHLL molecules of the invention, the linker sequence of Linker 3 is at least 15 amino acids. In other embodiments, Linker 3 is at least 20 amino acids. In other embodiments, Linker 3 is at least 25 amino acids. In other embodiments, Linker 3 is at least 30 amino acids. In other embodiments, Linker 3 is 15-30 amino acids. In other embodiments, Linker 3 is 15, 16, 17, 18, 19, 20, 21, 22, 23, 24, 25, 26, 27, 28, 29, or 30 amino acids. In yet other embodiments, Linker 3 is greater than 30 amino acids.

In certain embodiments in the context of the HHLL molecules of the invention, the linker sequence of Linker 4 is at least 5 amino acids. In other embodiments, Linker 4 is at least 10 amino acids. In other embodiments, Linker 4 is at least 15 amino acids. In other embodiments, Linker 4 is at least 20 amino acids. In other embodiments, Linker 4 is at least 25 amino acids. In other embodiments, Linker 4 is at least 30 amino acids. In other embodiments, Linker 4 is 5-30 amino acids. In other embodiments, Linker 4 is 5, 6, 7, 8, 9, 10, 11, 12, 13, 14, 15, 16, 17, 18, 19, 20, 21, 22, 23, 24, 25, 26, 27, 28, 29, or 30 amino acids. In yet other embodiments, Linker 4 is greater than 30 amino acids.

In certain embodiments in the context of the HHLL molecules of the invention, the linker sequences and positions are set forth in the following Table 1, with linker positions corresponding to those set forth in FIG. 1, and with Linker 4 being optionally used if an Fc region is also attached to the HHLL molecule.

TABLE 1
Linkers

| | SEQ | | SEQ | | SEQ | | SEQ |
|---|---|---|---|---|---|---|---|
| Linker 1 | ID NO: | Linker 2 | ID NO: | Linker 3 | ID NO: | Linker 4 | ID NO: |
| (GGGGS)₂ | 2 | (GGGGS)₃ | 3 | (GGGGS)₃ | 3 | GGGG | 100 |
| (GGGGS)₄ | 4 | (GGGGS)₄ | 4 | (GGGGS)₄ | 4 | GGGG | 100 |
| (GGGGS)₅ | 5 | (GGGGS)₅ | 5 | (GGGGS)₅ | 5 | GGGG | 100 |
| (GGGGS)₃ | 3 | (GGGGS)₅ | 5 | (GGGGS)₅ | 5 | GGGG | 100 |
| (GGGGS)₃ | 3 | (GGGGS)₃ | 3 | (GGGGS)₂ | 2 | GGGG | 100 |
| (GGGGS)₂₋₁₀ | 96 | (GGGGS)₃₋₁₀ | 98 | (GGGGS)₃₋₁₀ | 98 | (GGGG)₁₋₁₀ | 101 |
| (GGGGQ)₂ | 7 | (GGGGQ)₃ | 8 | (GGGGQ)₃ | 8 | GGGG | 100 |
| (GGGGQ)₄ | 9 | (GGGGQ)₄ | 9 | (GGGGQ)₄ | 9 | GGGG | 100 |
| (GGGGQ)₅ | 10 | (GGGGQ)₅ | 10 | (GGGGQ)₅ | 10 | GGGG | 100 |
| (GGGGQ)₃ | 8 | (GGGGQ)₅ | 10 | (GGGGQ)₅ | 10 | GGGG | 100 |
| (GGGGQ)₂₋₁₀ | 97 | (GGGGQ)₃₋₁₀ | 99 | (GGGGQ)₃₋₁₀ | 99 | (GGGG)₁₋₁₀ | 101 |

*numerical subscript indicates the number of repeats, e.g., (GGGGS)₂ =
GGGGSGGGGS (SEQ ID NO: 2)
Note that the 3-3-2 linker was purposefully designed with non-optimal lengths
to serve as a negative control.

### Protease Cleavage Sites

In certain therapeutic applications, it may be advantageous to design the bispecific binding construct in a manner such that it is only active in proximity to target cells or their local microenvironment. For example, in certain cancers, inflammatory diseases, fibrotic diseases, and neurodegenerative diseases that produce proteases into the microenvironment, the bispecific binding construct is then activated once present in the diseased cells microenvironment. See, e.g., Broder and Becker-Pauly (2013), Biochem. J. 450: pp.253-264. Also see, e.g., Metz et al. (2012), Protein Engineering, Design and Selection, Vol.25, Issue 10, pp.571-580. In this type of disease state, the bispecific binding construct can be activated in the presence of proteases produced by disease cells, but not in their absence. Thus, a bispecific binding construct as described herein can be specifically activated in a disease microenvironment and be less active or inactive in other areas of the body, which can result in fewer negative side effects experienced by the patient receiving the therapy.

Accordingly, in certain embodiments, the bispecific binding constructs comprise a protease cleavage site within the linkers that join certain domains, where this protease cleavage site can be cleaved by a protease that is produced by target cells, for example cancer cells or infected cells, or pathogens, and where this cleavage activates the molecule.

A “protease cleavage site” as meant herein, includes an amino acid sequence that can be cleaved by a protease, such as, for example, a metalloproteinase (e.g., a matrix metalloproteinase (MMP) such as MMP2, MMP9, MMP11, or others), a serine protease, a cysteine protease, a plasmin, or a plasminogen activator (such as urokinase-type plasminogen activator (u-PA) or tissue plasminogen activator (tPA)), fibroblast activation protein α (FAP α), or a furin among any others. Representative locations of protease cleavage sites within linkers are diagrammed in FIGS. 1 and 2 herein. Nonlimiting examples of amino acid sequences comprised by such protease cleavage sites include those listed in Table 2 herein.

In some embodiments, the protease cleavage sites can include, for example, sites cleaved by plasmin. The pro-enzyme plasminogen is activated by proteolytic cleavage by u-PA leading to its conversion to the active enzyme, plasmin. Plasmin, a serine protease, may play a role in metastasis due to its degradation of extracellular matrix and its activation of other enzymes, for example, type-IV collagenase. See, e.g., Kaneko et al. (2003), Cancer Sci. 94(1): 43-39.

The matrix metalloproteinases (MMPs) MMP-2 and MMP-9 are overexpressed in a variety of human tumors, including ovarian, breast, and prostate tumors, as well as in melanoma. Moreover, an association between aggressive tumor growth and high levels of MMP-2 and/or MMP-9 has been observed in both clinical and experimental studies. See, e.g., Roomi et al. (2009), Onc. Rep. 21: 1323-1333. An MMP-2 or MMP-9 cleavage site can be represented as P4-P3-P2-P1IP1′-P2′-P3′-P4′, where P1-P4 and P1′-P4′ are amino acids and the vertical line represents the cleavage site. Some generalizations can be made about an MMP-2 cleavage site. P1 is most likely to be glycine or proline. P2 is most likely to be proline, with alanine, valine, or isoleucine being somewhat less likely. P3 is mostly likely to be alanine, serine, or arginine. P4 is most likely to be alanine, glycine, asparagine, or serine. P1′ is most likely to be leucine, with isoleucine, phenylalanine, or tyrosine being somewhat less likely. P2′ is most likely to be lysine, with alanine, valine, isoleucine, or tyrosine being somewhat less likely. P3′ is most likely to be alanine, serine, or glycine. P4′ is most likely to be alanine, lysine, or aspartic acid. There are somewhat clearer preferences for MMP-9 cleavage sites. P4 is most likely to be glycine. P3 is most likely proline. P2 is most likely to be lysine. P1 is most likely to be glycine or proline. P1′ is most likely to be leucine, with isoleucine being somewhat less likely. P2′ is most likely to be lysine . P3′ is most likely to be glycine or alanine. P4′ is most likely to alanine, proline, or tyrosine. Any MMP-2 or MMP-9 cleavage site can be located within the bispecific binding constructs (e.g., in the linkers) described herein, including those disclosed in Table 2 or in, e.g., Metz et al. (2012), Protein Engineering, Design and Selection, Vol.25, Issue 10, pp.571-580 or e.g., Prudova et al. (2010), Mol. Cell. Proteomics 9(5): 894-911.

In some embodiments, the protease cleavage sites used in the linkers also include, for example, cleavage sites for the metalloproteases meprin a and meprin 13, which may be involved in diseases such as certain cancers, inflammatory bowel diseases, cystic fibrosis, kidney diseases, diabetic nephropathy, and dermal fibrotic tumors. The cleavage sites of meprins a and 13 are not limited to a single, defined sequence for each of these proteases. However, at certain amino acid positions relative to the cleavage site, there is a strong preference for one or a handful of specific amino acids. See, e.g., Becker-Pauly et al. (2011), Molecular and Cellular Proteomics 10(9):M111.009233. DOI:10.1074/mcp.M111.009233, the portions of which describe particular cleavage site, including the supplementary material, are incorporated herein by reference. A small selection of known cleavage sites for various proteases, including meprin a and meprin 13, are provided in Table 2 herein.

Higher-than-normal levels of u-PA are known to be associated with various cancers, including, for example colorectal cancer, breast cancer, monocytic and myelogenous leukemias, bladder cancer, thyroid cancer, liver cancer, gastric cancer, and cancers of the pleura, lung, pancreas, ovaries, and the head and neck. See, e.g., Skelly et al. (1997), Clin. Can. Res. 3: 1837-1840; Han et al. (2005), Oncol. Rep. 14(1): 105-112; Kaneko et al. (2003), Cancer Sci. 94(1): 43-49; Liu et al. (2001), J. Biol. Chem. 276(21): 17976-17984. In Table 2 herein a small sample of sites that can be cleaved by u-PA are reported. Accordingly, the bispecific binding constructs described herein can comprise a cleavage site for any serine protease, including u-PA and tissue plasminogen activator (tPA), and including any of those cleavage sites listed in Table 2.

Some cysteine proteases, such as cathepsin B, have been found to be overexpressed in tumor tissue and likely play a causative role in some cancers. See, e.g., Emmert-Buck et al. (1994), Am. J. Pathol. 145(6): 1285-1290; Biniosseek et al. (2011), J. Proteome Res. 10: 5363-5373. As with cleavage sites for meprin α and meprin β, there is a lot of heterogeneity in cathepsin B cleavage sites. A cleavage site for cathepsin B (as well as other proteases) can be represented as P3-P2-P1|P1′-P2′-P3′, where P1-P3 and P1′-P3′ are all amino acids and vertical line represents the cleavage site. Some generalizations apply to cathepsin B cleavage sites. P3 is most often G, F, L, or P (using one letter code for amino acids). P2 is most often A, V, Y, F, or I. P1 is most often G, A, M, Q, or T. P1′ is most often F, G, I, V, or L. P2′ is most often V, I, G, T, or A. P3′ is most often G. Further there is some subsite cooperatively. For example, if P2 is F, then P3 is most likely to be G and least likely to be L, and P1′ is most likely to be F and least likely to be L. This and other examples of subsite cooperativity are described in detail in Biniossek et al. (2011), J. Proteome Res. 10: 5363-5373. Accordingly, all cathepsin B cleavage sites, including without limitation those in Table 2 herein, can be comprised by the bispecific binding constructs described herein.

In some embodiments, the bispecific binding constructs comprise the protease cleavage site Gly-Gly-Pro-Leu-Gly-Met-Leu-Ser-Gln-Ser (SEQ ID NO: 45), Gly-Pro-Leu-Gly-Ile-Ala-Gly-Gln (SEQ ID NO: 44) or Ala-Val-Arg-Trp-Leu-Leu-Thr-Ala (SEQ ID NO: 102), which can be cleaved by metalloproteinases. Other examples of protease cleavage sites include Arg-Arg-Arg-Arg-Arg-Arg (SEQ ID NO: 54), which is cleaved by a furin.

Cleavage at the protease cleavage site can be assessed by various assays known in the art, e.g., by SDS-PAGE and/or Western blot. In certain embodiments, the binding constructs bind to a target more effectively when the protease cleavage sites are essentially completely cleaved, which can be assessed by, e.g., SDS-PAGE and/or Western blot.

TABLE 2
Examples of Protease Cleavage Sites

| Protease | Sequence of cleavage site* |
|---|---|
| meprin α | APMAIEGGG (SEQ ID NO: 17) |
| meprin β | EAQGIDKII (SEQ ID NO: 18) |
| | LAFSIDAGP (SEQ ID NO: 19) |
| | YVAIDAPK (SEQ ID NO: 20) |
| u-PA | SGRISA (SEQ ID NO: 21) |
| | GSGRISA (SEQ ID NO: 22) |
| | SGKISA (SEQ ID NO: 23) |
| u-PA | SGRISS (SEQ ID NO: 24) |
| | SGRIRA (SEQ ID NO: 25) |
| | SGRINA (SEQ ID NO: 26) |
| | SGRIKA (SEQ ID NO: 27) |
| tPA | QRGRISA (SEQ ID NO: 28) |
| cathepsin B | TQGIAAA (SEQ ID NO: 29) |
| | GAAIAAA (SEQ ID NO: 30) |
| | GAGIAAG (SEQ ID NO: 31) |
| | AAAIAAG (SEQ ID NO: 32) |
| | LCGIAAI (SEQ ID NO: 33) |
| | FAQIALG (SEQ ID NO: 34) |
| | LAAIANP (SEQ ID NO: 35) |
| | LLQIANP (SEQ ID NO: 36) |
| | LAAIANP (SEQ ID NO: 37) |
| | LYGIAQF (SEQ ID NO: 38) |
| | LSQIAQG (SEQ ID NO: 39) |
| | ASAIASG (SEQ ID NO: 40) |
| | FLGIASL (SEQ ID NO: 41) |
| | AYGIATG (SEQ ID NO: 42) |
| | LAQIATG (SEQ ID NO: 43) |
| MMP-2 | GPLGIIAGQ (SEQ ID NO: 44) |
| | GGPLGIMLSQS (SEQ ID NO: 45)** |
| | PLGILAG (SEQ ID NO: 46) |
| MMP-11 | AANILRN (SEQ ID NO: 47) |
| | AQAIYVK (SEQ ID NO: 48) |
| | AANIYMR (SEQ ID NO: 49) |
| | AAAILTR (SEQ ID NO: 50) |
| | AQNILMR (SEQ ID NO: 51) |
| | AANIYTK (SEQ ID NO: 52) |
| Furin | RRRRR (SEQ ID NO: 53) |
| | RRRRRR (SEQ ID NO: 54) |
| | GQSSRHRRAL (SEQ ID NO: 55) |

*vertical lines, when present, represent the predicted
cleavage site
**Note that this sequence is also cleaved by MMP-9

### Cysteine Clamps

A “cysteine clamp” involves the introduction of a cysteine into a polypeptide domain at a specific location, typically through replacing an existing amino acid at the specific location, so that when in proximity with another polypeptide domain, also having a cysteine introduced at a specific location, a disulfide bond (a “cysteine clamp”) may be formed between the two domains.

In some embodiments, a linker sequence comprising a protease cleavage site can result in a molecule that, once the protease cleavage site has been cleaved, does not yield the desired molecular structure due to a lack of a covalent link between appropriate polypeptide domains. Accordingly, in certain embodiments, covalent linkage is provided by one or more engineered disulfide bonds introduced at specified locations (a “cysteine clamp”). Nonlimiting examples of these cysteine clamps can be found in U.S. Pat. Appl. Publ. No. 2016/0193295A1, U.S. Pat. Appl. Publ. No. 2017/0306033A1, and U.S. Pat. Appl. Publ. No. 2018/0079790A1.

In certain embodiments, an antibody Fc domain may comprise the cysteine clamp(s), such as the CH2 and/or CH3 domains. See, for example, U.S. Pat. Appl. Publ. No. 2016/0193295A1. In a specific embodiment, an scFc comprises at least one cysteine clamp that results in a disulfide bond across both CH2 domains. In a further specific embodiment, an scFc comprises at least two cysteine clamps that results in a disulfide bond across both CH2 domains.

In certain embodiments, the amino acid residues where the CH2 sequence has been altered to create the cysteine clamp(s) may be selected from the following, where one or more amino acids are substituted with cysteine: R72C, V82C, R329C, R339C

In certain embodiments, specific pairs of residues are substituted such that they preferentially form a di-sulfide bond with each other, thus limiting or preventing di-sulfide bond scrambling. Nonlimiting examples of these specific pairs include, but are not limited to, 72 C-82 C, 329 C-339 C.

In other embodiments, a binding construct's VH and VL domains may comprise the cysteine clamp(s) to result in disulfide bond formation between the VH and VL domains. These cysteine clamps will stabilize the VH and VL domains in an antigen-binding configuration. See, for example, U.S. Pat. Appl. Publ. No. 2017/0306033A1.

In certain embodiments, the amino acid residues where the VH and VL sequence has been altered to create the cysteine clamp(s) may be selected from the following, where one or more amino acids are substituted with cysteine: Kabat VH44 VL100 for anti-MSLN and VH103 VL43 for anti-CD3.

In certain embodiments, specific pairs of residues are substituted such that they preferentially form a di-sulfide bond with each other, thus limiting or preventing di-sulfide bond scrambling. Nonlimiting examples of these specific pairs include, but are not limited to, MSLN VH44-VL100, anti-CD3 VH103-VL43.

### Amino Acid Sequences of Binding Regions

In the exemplary embodiments described herein, the bispecific binding constructs maintain desired binding to the various desired targets which results from their assuming the proper conformation to allow this binding. The immunoglobulin variable region comprises a VH and a VL domain, which associate to form the variable domain which binds the desired target.

The variable domains can be obtained from any immunoglobulin with the desired characteristics, and the methods to accomplish this are further described herein. In one embodiment, VH1 and VL1 associate and bind CDR, and VH2 and VL2 associate and bind a different target. In another embodiment, the VH2 and VL2 bind CD3ϵ and the VH1 and VL1 bind a different target.

In another embodiment, the light-chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a light chain variable domain listed herein.

In another embodiment, the light chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99% or 100% identical to the polynucleotide sequence listed herein. In another embodiment, the light chain variable domain comprises a sequence of amino acids that is encoded by a polynucleotide that hybridizes under moderately stringent conditions to the complement of a polynucleotide that encodes a light chain variable domain selected from the sequences listed herein. In another embodiment, the light chain variable domain comprises a sequence of amino acids that is encoded by a polynucleotide that hybridizes under stringent conditions to the complement of a polynucleotide that encodes a light chain variable domain selected from the group consisting of the sequences listed herein.

In another embodiment, the heavy chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a heavy chain variable domain selected from the sequences listed herein. In another embodiment, the heavy chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99% or 100% identical to a nucleotide sequence that encodes a heavy chain variable domain selected from the sequences listed herein. In another embodiment, the heavy chain variable domain comprises a sequence of amino acids that is encoded by a polynucleotide that hybridizes under moderately stringent conditions to the complement of a polynucleotide that encodes a heavy chain variable domain selected from the sequences listed herein. In another embodiment, the heavy chain variable domain comprises a sequence of amino acids that is encoded by a polynucleotide that hybridizes under stringent conditions to the complement of a polynucleotide that encodes a heavy chain variable domain selected from the sequences listed herein.

### Substitutions

It will be appreciated that a bispecific binding construct of the present invention may have at least one amino acid substitution, providing that the bispecific binding construct retains the same or better desired binding specificity (e.g., binding to CD3). Therefore, modifications to the bispecific binding construct structures are encompassed within the scope of the invention. In one embodiment, the bispecific binding construct comprises sequences that each independently differ by 5, 4, 3, 2, 1, or 0 single amino acid additions, substitutions, and/or deletions from a CDR sequence of those set forth herein. As used herein, a CDR sequence that differs by no more than a total of, for example, four amino acid additions, substitutions and/or deletions from a CDR sequence set forth herein refers to a sequence with 4, 3, 2, 1 or 0 single amino acid additions, substitutions, and/or deletions compared with the sequences set forth herein. These may include amino acid substitutions, which may be conservative or non-conservative that do not destroy the desired binding capability of a binding construct. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions may involve the exchange of a member of one class of amino acids or amino acid mimetics for a member from another class with different physical properties (e.g. size, polarity, hydrophobicity, charge). In certain embodiments, such substituted residues may be introduced into regions of a human antibody that are homologous with non-human antibodies, or into the non-homologous regions of the molecule.

Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A skilled artisan will be able to determine suitable variants of the bispecific binding construct as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides as has been describe herein. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

In some embodiments, one skilled in the art may identify residues that may be changed that result in enhanced properties as desired. For example, an amino acid substitution (conservative or non-conservative) may result in enhanced binding affinity to a desired target.

One skilled in the art can also analyze the three-dimensional structure and amino acid sequence in relation to that structure in similar polypeptides. In view of such information, one skilled in the art may predict the alignment of amino acid residues of an antibody with respect to its three-dimensional structure. In certain embodiments, one skilled in the art may choose not to make radical changes to amino acid residues predicted to be on the surface of the protein, since such residues may be involved in important interactions with other molecules. A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The growth of the protein structural database (PDB) has provided enhanced predictability of secondary structure, including the potential number of folds within a polypeptide's or protein's structure. See Holm et al., Nucl. Acid. Res., 27(1):244-247 (1999). Additional methods of predicting secondary structure include “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, variants of the bispecific binding construct include glycosylation variants wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional antibody variants include cysteine variants wherein one or more cysteine residues are deleted from or substituted for another amino acid (e.g., serine) as compared to the parent amino acid sequence. Cysteine variants may be useful when antibodies or bispecific binding constructs must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies or bispecific binding constructs to the target of interest, or to increase or decrease the affinity of the antibodies or bispecific binding constructs to the target of interest described herein.

According to certain embodiments, desired amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

### Half-Life Extension and Fc Regions

In certain embodiments, it is desirable to extend the in vivo half-life of the bispecific binding constructs of the invention. This can be accomplished by including a half-life extending moiety as part of the bispecific binding construct. Nonlimiting examples of half-life extending moieties include an Fc polypeptide, albumin, an albumin fragment, a moiety that binds to albumin or to the neonatal Fc receptor (FcRn), a derivative of fibronectin that has been engineered to bind albumin or a fragment thereof, a peptide, a single domain protein fragment, or other polypeptide that can increase serum half-life. In alternate embodiments, a half-life-extending moiety can be a non-polypeptide molecule such as, for example, polyethylene glycol (PEG).

The term “Fc polypeptide” as used herein includes native and mutein forms of polypeptides derived from the Fc region of an antibody. Truncated forms of such polypeptides containing the hinge region that promotes dimerization also are included. In addition to other properties described herein, polypeptides comprising Fc moieties offer the advantage of purification by affinity chromatography over, e.g., Protein A or Protein G columns.

In certain embodiments, the half-life extending moiety is an Fc region of an antibody. In certain embodiments, the Fc region is located at the N-terminal end of the HHLL bispecific binding construct. In other embodiments, the Fc region is located at the C-terminal end of the HHLL bispecific binding construct. In yet other embodiments, the Fc region can be located between the VH and VL subunits as shown in FIG. 2 herein. There can be, but need not be, a linker between the HHLL bispecific binding construct and the Fc region. As explained herein, an Fc polypeptide chain may comprise all or part of a hinge region followed by a CH2 and a CH3 region. The Fc polypeptide chain can be of mammalian (for example, human, mouse, rat, rabbit, dromedary, or new or old world monkey), avian, or shark origin. In addition, as explained herein, an Fc polypeptide chain can include a limited number of alterations. For example, an Fc polypeptide chain can comprise one or more heterodimerizing alterations, one or more alteration that inhibits or enhances binding to FcγR, or one or more alterations that increase binding to FcRn.

In a specific embodiment, the Fc utilized for half-life extension is a single chain Fc (“scFc”).

In some embodiments the amino acid sequences of the Fc polypeptides can be mammalian, for example a human, amino acid sequences. The isotype of the Fc polypeptide can be IgG, such as IgG1, IgG2, IgG3, or IgG4, IgA, IgD, IgE, or IgM. Table 3 below shows an alignment of the amino acid sequences of human IgG1, IgG2, IgG3, and IgG4 Fc polypeptide chains.

Sequences of human IgG1, IgG2, IgG3, and IgG4 Fc polypeptides that could be used are provided in SEQ ID NOs: 56 - 59. Variants of these sequences containing one or more heterodimerizing alterations, one or more Fc alteration that extends half life, one or more alteration that enhances ADCC, and/or one or more alteration that inhibits Fc gamma receptor (FcγR) binding are also contemplated, as are other close variants containing not more than 10 deletions, insertions, or substitutions of a single amino acid per 100 amino acids of sequence.

TABLE 3
Amino acid sequences of human IgGFc polypeptide chains

| | |
|---|---|
| IgG1 | ----------------------------------------------- |
| IgG2 | ----------------------------------------------- |
| IgG3 | ELKTPLGDTTHTCPRCPEPKSCDTPPPCPRCPEPKSCDTPPPCPRCP |
| IgG4 | ----------------------------------------------- |
| | 225 235 245 255 265 275 |
| | * * * * * * |
| IgG1 | EPKSCDKTHTCPPCPAPELLGGPSVFLFPPKPKDTLMISRTPEVTCVVVDVSHEDPEVKF |
| IgG2 | ERKCCVE---CPPCPAPPVA-GPSVFLFPPKPKDTLMISRTPEVTCVVVDVSHEDPEVQF |
| IgG3 | EPKSCDTPPPCPRCPAPELLGGPSVFLFPPKPKDTLMISRTPEVTCVVVDVSHEDPEVQF |
| IgG4 | ESKYG---PPCPSCPAPEFLGGPSVFLFPPKPKDTLMISRTPEVTCVVVDVSQEDPEVQF |
| | 285 295 305 315 325 335 |
| | * * * * * * |
| IgG1 | NWYVDGVEVHNAKTKPREEQYNSTYRVVSVLTVLHQDWLNGKEYKCKVSNKALPAPIEKT |
| IgG2 | NWYVDGMEVHNAKTKPREEQFNSTFRVVSVLTVVHQDWLNGKEYKCKVSNKGLPAPIEKT |
| IgG3 | KWYVDGVEVHNAKTKPREEQYNSTFRVVSVLTVLHQDWLNGKEYKCKVSNKALPAPIEKT |
| IgG4 | NWYVDGVEVHNAKTKPREEQFNSTYRVVSVLTVLHQDWLNGKEYKCKVSNKGLPSSIEKT |
| | 345 355 365 375 385 395 |
| | * * * * * * |
| IgG1 | ISKAKGQPREPQVYTLPPSREEMTKNQVSLTCLVKGFYPSDIAVEWESNGQPENNYKTTP |
| IgG2 | ISKTKGQPREPQVYTLPPSREEMTKNQVSLTCLVKGFYPSDIAVEWESNGQPENNYKTTP |
| IgG3 | ISKTKGQPREPQVYTLPPSREEMTKNQVSLTCLVKGFYPSDIAVEWESSGQPENNYNTTP |
| IgG4 | ISKAKGQPREPQVYTLPPSQEEMTKNQVSLTCLVKGFYPSDIAVEWESNGQPENNYKTTP |
| | 405 415 425 435 445 |
| | * * * * * |
| IgG1 | PVLDSDGSFFLYSKLTVDKSRWQQGNVFSCSVMHEALHNHYTQKSLSLSPGK (SEQ ID NO: 56) |
| IgG2 | PMLDSDGSFFLYSKLTVDKSRWQQGNVFSCSVMHEALHNHYTQKSLSLSPGK (SEQ ID NO: 57) |
| IgG3 | PMLDSDGSFFLYSKLTVDKSRWQQGNIFSCSVMHEALHNRFTQKSLSLSPGK (SEQ ID NO: 58) |
| IgG4 | PVLDSDGSFFLYSRLTVDKSRWQEGNVFSCSVMHEALHNHYTQKSLSLSLGK (SEQ ID NO: 59) |

The numbering shown in Table 3 is according the EU system of numbering, which is based on the sequential numbering of the constant region of an IgG1 antibody. Edelman et al. (1969), Proc. Natl. Acad. Sci. 63: 78-85. Thus, it does not accommodate the additional length of the IgG3 hinge well. It is nonetheless used here to designate positions in an Fc region because it is still commonly used in the art to refer to positions in Fc regions. The hinge regions of the IgG1, IgG2, and IgG4 Fc polypeptides extend from about position 216 to about 230. It is clear from the alignment that the IgG2 and IgG4 hinge regions are each three amino acids shorter than the IgG1 hinge. The IgG3 hinge is much longer, extending for an additional 47 amino acids upstream. The CH2 region extends from about position 231 to 340, and the CH3 region extends from about position 341 to 447.

Naturally occurring amino acid sequences of Fc polypeptides can be varied slightly. Such variations can include no more than 10 insertions, deletions, or substitutions of a single amino acid per 100 amino acids of sequence of a naturally occurring Fc polypeptide chain. If there are substitutions, they can be conservative amino acid substitutions, as defined herein. The Fc polypeptides on the first and second polypeptide chains can differ in amino acid sequence. In some embodiments, they can include “heterodimerizing alterations,” for example, charge pair substitutions, as defined herein, that facilitate heterodimer formation. Further, the Fc polypeptide portions of the PABP can also contain alterations that inhibit or enhance FcγR binding. Such mutations are described herein and in Xu et al. (2000), Cell Immunol. 200(1): 16-26, the relevant portions of which are incorporated herein by reference. The Fc polypeptide portions can also include an “Fc alteration that extends half life,” as described herein, including those described in, e.g., U.S. Pat. Nos. 7,037,784, 7,670,600, and 7,371,827, US Patent Application Publication 2010/0234575, and International Application PCT/US2012/070146, the relevant portions of all of which are incorporated herein by reference. Further, an Fc polypeptide can comprise “alterations that enhance ADCC,” as defined herein.

Another suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

The effector function of an antibody or binding construct can be increased, or decreased, by introducing one or more mutations into the Fc. Embodiments of the invention include IL-2 mutein Fc fusion proteins having an Fc engineered to increase effector function (U.S. Pat. No. 7,317,091 and Strohl, Curr. Opin. Biotech., 20:685-691, 2009; both incorporated herein by reference in its entirety). For certain therapeutic indications, it may be desirable to increase effector function. For other therapeutic indications, it may be desirable to decrease effector function.

Exemplary IgG1 Fc molecules having increased effector function include those having the following substitutions:
S239D/I332E
S239D/A330S/I332E
S239D/A330L/I332E
S298A/D333A/K334A
P2471/A339D
P2471/A339Q
D280H/K290S
D280H/K290S/S298D
D280H/K290S/S298V
F243L/R292P/Y300L
F243L/R292P/Y300L/P396L
F243L/R292P/Y300L/V3051/P396L
G236A/S239D/I332E
K326A/E333A
K326W/E333S
K290E/S298G/T299A
K290N/S298G/T299A
K290E/S298G/T299A/K326E
K290N/S298G/T299A/K326E

Another method of increasing effector function of IgG Fc-containing proteins is by reducing the fucosylation of the Fc. Removal of the core fucose from the biantennary complex-type oligosachharides attached to the Fc greatly increased ADCC effector function without altering antigen binding or CDC effector function. Several ways are known for reducing or abolishing fucosylation of Fc-containing molecules, e.g., antibodies. These include recombinant expression in certain mammalian cell lines including a FUT8 knockout cell line, variant CHO line Lec13, rat hybridoma cell line YB2/0, a cell line comprising a small interfering RNA specifically against the FUT8 gene, and a cell line coexpressing α-1,4-N-acetylglucosaminyltransferase III and Golgi α-mannosidase II. Alternatively, the Fc-containing molecule may be expressed in a non-mammalian cell such as a plant cell, yeast, or prokaryotic cell, e.g., E. coli.

In certain embodiments of the invention, the bispecific binding constructs comprise an Fc engineered to decrease effector function. Exemplary Fc molecules having decreased effector function include those having the following substitutions:
N297A or N297Q (IgG1)
L234A/L235A (IgG1)
V234A/G237A (IgG2)
L235A/G237A/E318A (IgG4)
H268Q/V309L/A330S/A331S (IgG2)
C220S/C226S/C229S/P238S (IgG1)
C226S/C229S/E233P/L234V/L235A (IgG1)
L234F/L235E/P331S (IgG1)
S267E/L328F (IgG1)

It is known that human IgG1 has a glycosylation site at N297 (EU numbering system) and glycosylation contributes to the effector function of IgG1 antibodies. An exemplary IgG1 sequence is provided in SEQ ID NO: 36. N297 can be mutated to make aglycosylated antibodies. For example, mutations can substitute N297 with amino acids that resemble asparagine in physiochemical nature such as glutamine (N297Q), or with alanine (N297A), which mimics asparagines without polar groups.

In certain embodiments, mutation of amino acid N297 of human IgG1 to glycine, i.e., N297G, provides far superior purification efficiency and biophysical properties over other amino acid substitutions at that residue. See, for example, U.S. Pat. Nos. 9,546,203 and 10,093,711. In a specific embodiment, the bispecific binding constructs of the invention comprise a human IgG1 Fc having an N297G substitution.

A bispecific binding construct of the invention comprising a human IgG1 Fc having the N297G mutation may also comprise further insertions, deletions, and substitutions. In certain embodiments the human IgG1 Fc comprises the N297G substitution and is at least 90% identical, at least 91% identical, at least 92% identical, at least 93% identical, at least 94% identical, at least 95% identical, at least 96% identical, at least 97% identical, at least 98% identical, or at least 99% identical to the amino acid sequence set forth in SEQ ID NO: 36. In a particularly preferred embodiment, the C-terminal lysine residue is substituted or deleted.

In certain instances, aglycosylated IgG1 Fc-containing molecules can be less stable than glycosylated IgG1 Fc-containing molecules. Accordingly, the Fc region may be further engineered to increase the stability of the aglycosylated molecule. In some embodiments, one or more amino acids are substituted to cysteine so to form di-sulfide bonds in the dimeric state. In specific embodiments, residues V259, A287, R292, V302, L306, V323, or 1332 of the amino acid sequence set forth in SEQ ID NOs: 56-59 may be substituted with cysteine. In other embodiments, specific pairs of residues are substitution such that they preferentially form a di-sulfide bond with each other, thus limiting or preventing di-sulfide bond scrambling. In specific embodiments, pairs include, but are not limited to, A287C and L306C, V259C and L306C, R292C and V302C, and V323C and I332C.

As discussed herein in the Linker section, in certain embodiments, the bispecific binding constructs of the invention comprise a linker between the Fc and the HHLL bispecific binding construct, specifically, linking the Fc to the VL2. In certain embodiments, one or more copies of a peptide consisting of GGGGS (SEQ ID NO: 1), GGNGT (SEQ ID NO: 15), or YGNGT (SEQ ID NO: 16) between the Fc and the HHLL polypeptide. In some embodiments, the polypeptide region between the Fc region and the HHLL polypeptide comprises a single copy of GGGGS (SEQ ID NO: 1), GGNGT (SEQ ID NO: 15), or YGNGT (SEQ ID NO: 16). In certain embodiments, the linkers GGNGT (SEQ ID NO: 15) or YGNGT (SEQ ID NO: 16) are glycosylated when expressed in the appropriate cells and such glycosylation may help stabilize the protein in solution and/or when administered in vivo. Accordingly, in certain embodiments, a bispecific binding construct of the invention comprises a glycosylated linker between the Fc region and the HHLL polypeptide.

### Nucleic Acids Encoding the Bispecific Binding Constructs

In another embodiment, the present invention provides isolated nucleic acid molecules that encode the bispecific binding constructs of the present invention. In addition, provided are vectors comprising the nucleic acids, cell comprising the nucleic acids, and methods of making the bispecific binding constructs of the invention. The nucleic acids comprise, for example, polynucleotides that encode all or part of bispecific binding construct, for example, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The nucleic acids can be any length as appropriate for the desired use or function, and can comprise one or more additional sequences, for example, regulatory sequences, and/or be part of a larger nucleic acid, for example, a vector. The nucleic acids can be single-stranded or double-stranded and can comprise RNA and/or DNA nucleotides, and artificial variants thereof (e.g., peptide nucleic acids).

Nucleic acids encoding polypeptides (e.g., heavy or light chain, variable domain only, or full length) may be isolated from B-cells of mice that have been immunized with antigen. The nucleic acid may be isolated by conventional procedures such as polymerase chain reaction (PCR).

Nucleic acid sequences encoding the variable regions of the heavy and light chain variable regions are included herein. The skilled artisan will appreciate that, due to the degeneracy of the genetic code, each of the polypeptide sequences disclosed herein is encoded by a large number of other nucleic acid sequences. The present invention provides each degenerate nucleotide sequence encoding each bispecific binding construct of the invention.

The invention further provides nucleic acids that hybridize to other nucleic acids under particular hybridization conditions. Methods for hybridizing nucleic acids are well-known in the art. See, e.g., Current Protocols in Molecular Biology, John Wiley & Sons, N.Y. (1989), 6.3.1-6.3.6. As defined herein, for example, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 98 or 99% identical to each other typically remain hybridized to each other. The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. Changes can be introduced by mutation into a nucleic acid, thereby leading to changes in the amino acid sequence of a polypeptide (e.g., a bispecific binding construct) that it encodes. Mutations can be introduced using any technique known in the art. In one embodiment, one or more particular amino acid residues are changed using, for example, a site-directed mutagenesis protocol. In another embodiment, one or more randomly selected residues is changed using, for example, a random mutagenesis protocol. However, it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. In one embodiment, a nucleotide sequence provided herein for of the binding constructs of the present invention, or a desired fragment, variant, or derivative thereof, is mutated such that it encodes an amino acid sequence comprising one or more deletions or substitutions of amino acid residues that are shown herein for the light chains of the binding constructs of the present invention or the heavy chains of the binding constructs of the present invention to be residues where two or more sequences differ. In another embodiment, the mutagenesis inserts an amino acid adjacent to one or more amino acid residues shown herein for the light chains of the binding constructs of the present invention or the heavy chains of the binding constructs of the present invention to be residues where two or more sequences differ. Alternatively, one or more mutations can be introduced into a nucleic acid that selectively change the biological activity of a polypeptide that it encodes.

In another embodiment, the present invention provides vectors comprising a nucleic acid encoding a polypeptide of the invention or a portion thereof. Examples of vectors include, but are not limited to, plasmids, viral vectors, non-episomal mammalian vectors and expression vectors, for example, recombinant expression vectors.

The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems (see id.). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

In another embodiment, the present invention provides host cells into which a recombinant expression vector of the invention has been introduced. A host cell can be any prokaryotic cell or eukaryotic cell. Prokaryotic host cells include gram negative or gram positive organisms, for example E. coli or bacilli. Higher eukaryotic cells include insect cells, yeast cells, and established cell lines of mammalian origin. Examples of suitable mammalian host cell lines include Chinese hamster ovary (CHO) cells or their derivatives such as Veggie CHO and related cell lines which grow in serum-free media (see Rasmussen et al., 1998, Cytotechnology 28:31) or CHO strain DXB-11, which is deficient in DHFR (see Urlaub et al., 1980, Proc. Natl. Acad. Sci. USA 77:4216-20). Additional CHO cell lines include CHO-K1 (ATCC#CCL-61), EM9 (ATCC #CRL-1861), and UV20 (ATCC #CRL-1862). Additional host cells include the COS-7 line of monkey kidney cells (ATCC CRL 1651) (see Gluzman et al., 1981, Cell 23:175), L cells, C127 cells, 3T3 cells (ATCC CCL 163), AM-1/D cells (described in U.S. Pat. No. 6,210,924), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Appropriate cloning and expression vectors for use with bacterial, fungal, yeast, and mammalian cellular hosts are described by Pouwels et al. (Cloning Vectors: A Laboratory Manual, Elsevier, New York, 1985).

Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Additional selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

The transformed cells can be cultured under conditions that promote expression of the polypeptide, and the polypeptide recovered by conventional protein purification procedures. Polypeptides contemplated for use herein include substantially homogeneous recombinant mammalian polypeptides substantially free of contaminating endogenous materials.

Cells containing the nucleic acid encoding the bispecific binding constructs of the present invention also include hybridomas. The production and culturing of hybridomas are discussed herein.

In some embodiments, a vector comprising a nucleic acid molecule as described herein is provided. In some embodiments, the invention comprises a host cell comprising a nucleic acid molecule as described herein.

In some embodiments, a nucleic acid molecule encoding the bispecific binding constructs as described herein is provided.

In some embodiments, a pharmaceutical composition comprising at least one bispecific binding construct described herein is provided.

### Methods of Producing

The bispecific binding constructs of the invention can be produced by any method known in the art for the synthesis of proteins (e.g., antibodies), in particular, by chemical synthesis or preferably, by recombinant expression techniques.

Recombinant expression of the bispecific binding constructs requires construction of an expression vector containing a polynucleotide that encodes the bispecific binding construct. Once a polynucleotide encoding the bispecific binding construct has been obtained, the vector for the production of the bispecific binding construct may be produced by recombinant DNA technology. An expression vector is constructed containing the bispecific binding construct coding sequences and appropriate transcriptional and translational control signals. These methods include, for example, in vitro recombinant DNA techniques, synthetic techniques, and in vivo genetic recombination.

The expression vector is transferred to a host cell by conventional techniques and the transfected cells are then cultured by conventional techniques to produce a bispecific binding construct of the invention.

A variety of host-expression vector systems may be utilized and readily adapted to express the bispecific binding constructs of the invention. Such host-expression systems represent vehicles by which the coding sequences of interest may be produced and subsequently purified, but also represent cells which may, when transformed or transfected with the appropriate nucleotide coding sequences, express a molecule of the invention in situ. Bacterial cells such as E. coli, and eukaryotic cells are commonly used for the expression of a recombinant antibody molecule, especially for the expression of whole recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, COS, 293, 3T3, or myeloma cells.

For long-term, high-yield production of recombinant proteins, stable expression is preferred. For example, cell lines which stably express the molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the molecule. Such engineered cell lines may be particularly useful in screening and evaluation of compounds that interact directly or indirectly with the molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk, hgprt or aprt-cells, respectively. Also, antimetabolite resistance can be used as the basis of selection for the following genes: dhfr, which confers resistance to methotrexate (Wigler et al., Proc. Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of a molecule can be increased by vector amplification (for a review, see Bebbington and Hentschel, “The use of vectors based on gene amplification for the expression of cloned genes in mammalian cells” (DNA Cloning, Vol. 3. Academic Press, New York, 1987)). When a marker in the vector system expressing molecule is amplifiable, increase in the level of inhibitor present in culture of host cell will increase the number of copies of the marker gene. Since the amplified region is associated with the antibody gene, production of the molecule will also increase (Crouse et al., Mol. Cell. Biol. 3:257 (1983)).

The host cell may be co-transfected with multiple expression vectors of the invention. The vectors may contain identical selectable markers which enable equal expression of the expressed polypeptides. Alternatively, a single vector may be used which encodes, and is capable of expressing, for example, the polypeptides of the invention. The coding sequences may comprise cDNA or genomic DNA.

Once a molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and size-exclusion chromatography), centrifugation, differential solubility, or by any other standard technique for the purification of proteins. In addition, the binding constructs of the present invention or fragments thereof can be fused to heterologous polypeptide sequences described herein or otherwise known in the art, to facilitate purification. The purification techniques may be varied, depending on whether an Fc region (e.g., an scFC) is attached to the bispecific binding constructs of the invention.

In some embodiments, the present invention encompasses binding constructs recombinantly fused or chemically conjugated (including both covalently and non-covalently conjugations) to a polypeptide. Fused or conjugated binding constructs of the present invention may be used for ease in purification. See e.g., Harbor et al., supra, and PCT publication WO 93/21232; EP 439,095; Naramura et al., Immunol. Lett. 39:91-99 (1994); U.S. Pat. No. 5,474,981; Gillies et al., Proc. Natl. Acad. Sci. 89:1428-1432 (1992); Fell et al., J. Immunol. 146:2446-2452 (1991).

Moreover, the binding constructs or fragments thereof of the present invention can be fused to marker sequences, such as a peptide to facilitate purification. In preferred embodiments, the marker amino acid sequence is a hexa-histidine peptide (SEQ ID NO: 103), such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine (SEQ ID NO: 103) provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

### Generation of Bispecific Binding Constructs

The bispecific binding constructs of the invention, in a general sense, are constructed by selecting VH and VL regions from desired antibodies and linking them using polypeptide linkers as described herein to form the HHLL bispecific binding construct, optionally with an Fc region attached. More specifically, the nucleic acids encoding the VH, VL and linkers, and optionally the Fc, are combined to create the HHLL nucleic acid constructs that encode the bispecific binding constructs of the invention.

### Generation of Antibodies

In certain embodiments, prior to generation of the bispecific binding constructs of the invention, monospecific antibodies are first generated with binding specificities to desired targets.

Antibodies to be used to generate the bispecific binding molecules of the invention may be prepared by techniques that are well known to those skilled in the art. For example, by immunizing an animal (e.g., a mouse or rat or rabbit) and then by immortalizing spleen cells harvested from the animal after completion of the immunization schedule. The spleen cells can be immortalized using any technique known in the art, e.g., by fusing them with myeloma cells to produce hybridomas. See, for example, Antibodies; Harlow and Lane, Cold Spring Harbor Laboratory Press, 1st Edition, e.g. from 1988, or 2nd Edition, e.g. from 2014).

In one embodiment, a humanized monoclonal antibody to be used to generate the bispecific binding molecules of the invention comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557, Padlan et al., 1995, FASEB J. 9:133-39, Tamura et al., 2000, J. Immunol. 164:1432-41, Zhang, W., et al., Molecular Immunology. 42(12):1445-1451, 2005; Hwang W. et al., Methods. 36(1):35-42, 2005; Dall'Acqua W F, et al., Methods 36(1):43-60, 2005; and Clark, M., Immunology Today. 21(8):397-402, 2000.

An antibody of the present invention may also be a fully human monoclonal antibody to be used to generate the bispecific binding molecules of the invention. Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue.

Antibodies produced in the animal incorporate human immunoglobulin polypeptide chains encoded by the human genetic material introduced into the animal. In one embodiment, a non-human animal, such as a transgenic mouse, is immunized with a suitable immunogen.

Examples of techniques for production and use of transgenic animals for the production of human or partially human antibodies are described in U.S. Pat. Nos. 5,814,318, 5,569,825, and 5,545,806, Davis et al., Production of human antibodies from transgenic mice in Lo, ed. Antibody Engineering: Methods and Protocols, Humana Press, NJ:191-200 (2003), Kellermann et al., 2002, Curr Opin Biotechnol. 13:593-97, Russel et al., 2000, Infect Immun. 68:1820-26, Gallo et al., 2000, Eur J Immun. 30:534-40, Davis et al., 1999, Cancer Metastasis Rev. 18:421-25, Green, 1999, J Immunol Methods. 231:11-23, Jakobovits, 1998, Advanced Drug Delivery Reviews 31:33-42, Green et al., 1998, J Exp Med. 188:483-95, Jakobovits A, 1998, Exp. Opin. Invest. Drugs. 7:607-14, Tsuda et al., 1997, Genomics. 42:413-21, Mendez et al., 1997, Nat Genet. 15:146-56, Jakobovits, 1994, Curr Biol. 4:761-63, Arbones et al., 1994, Immunity. 1:247-60, Green et al., 1994, Nat Genet. 7:13-21, Jakobovits et al., 1993, Nature. 362:255-58, Jakobovits et al., 1993, Proc Natl Acad Sci U S A. 90:2551-55. Chen, J., M. Trounstine, F. W. Alt, F. Young, C. Kurahara, J. Loring, D. Huszar. “Immunoglobulin gene rearrangement in B-cell deficient mice generated by targeted deletion of the JH locus.” International Immunology 5 (1993): 647-656, Choi et al., 1993, Nature Genetics 4: 117-23, Fishwild et al., 1996, Nature Biotechnology 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Internal Review of Immunology 13: 65-93, Neuberger, 1996, Nature Biotechnology 14: 826, Taylor et al., 1992, Nucleic Acids Research 20: 6287-95, Taylor et al., 1994, International Immunology 6: 579-91, Tomizuka et al., 1997, Nature Genetics 16: 133-43, Tomizuka et al., 2000, Proceedings of the National Academy of Sciences USA 97: 722-27, Tuaillon et al., 1993, Proceedings of the National Academy of Sciences USA 90: 3720-24, and Tuaillon et al., 1994, Journal of Immunology 152: 2912-20.; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N.Y. Acad. Sci. 764:525-35. In addition, protocols involving the XenoMouse® (Abgenix, now Amgen, Inc.) are described, for example in U.S. 05/0118643 and WO 05/694879, WO 98/24838, WO 00/76310, and U.S. Pat. No. 7,064,244.

Lymphoid cells from the immunized transgenic mice are fused with myeloma cells for example to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in such fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and 5194/5XX0 Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells but not unfused myeloma cells. One selection media is HAT (hypoxanthine, aminopterin, thymidine). After a sufficient time, usually about one to two weeks, colonies of cells are observed. Single colonies are isolated, and antibodies produced by the cells may be tested for binding activity to desired targets using any one of a variety of immunoassays known in the art and described herein. The hybridomas are cloned (e.g., by limited dilution cloning or by soft agar plaque isolation) and positive clones that produce an antibody specific to a desired target is selected and cultured. The monoclonal antibodies from the hybridoma cultures may be isolated from the supernatants of hybridoma cultures. Thus, the present invention provides hybridomas that comprise polynucleotides encoding the bispecific binding constructs of the invention in the chromosomes of the cell. These hybridomas can be cultured according to methods described herein and known in the art.

Another method for generating human antibodies to be used to generate the bispecific binding molecules of the invention includes immortalizing human peripheral blood cells by EBV transformation. See, e.g., U.S. Pat. No. 4,464,456. Such an immortalized B-cell line (or lymphoblastoid cell line) producing a monoclonal antibody that specifically binds to a desired target can be identified by immunodetection methods as provided herein, for example, an ELISA, and then isolated by standard cloning techniques. The stability of the lymphoblastoid cell line producing an antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B-cells with antigen, followed by fusion of primed B-cells with a heterohybrid fusion partner. See, e.g., Boerner et al., 1991 J. Immunol. 147:86-95.

In certain embodiments, a B-cell that is producing a desired antibody is selected and the light chain and heavy chain variable regions are cloned from the B-cell according to molecular biology techniques known in the art (WO 92/02551; U.S. Pat. No. 5,627,052; Babcook et al., Proc. Natl. Acad. Sci. USA 93:7843-48 (1996)) and described herein. B-cells from an immunized animal may be isolated from the spleen, lymph node, or peripheral blood sample by selecting a cell that is producing a desired antibody. B-cells may also be isolated from humans, for example, from a peripheral blood sample. Methods for detecting single B-cells that are producing an antibody with the desired specificity are well known in the art, for example, by plaque formation, fluorescence-activated cell sorting, in vitro stimulation followed by detection of specific antibody, and the like. Methods for selection of specific antibody-producing B-cells include, for example, preparing a single cell suspension of B-cells in soft agar that contains antigen. Binding of the specific antibody produced by the B-cell to the antigen results in the formation of a complex, which may be visible as an immunoprecipitate. After the B-cells producing the desired antibody are selected, the specific antibody genes may be cloned by isolating and amplifying DNA or mRNA according to methods known in the art and described herein and can be used to generate the bispecific binding molecules of the invention.

An additional method for obtaining antibodies to be used to generate the bispecific binding molecules of the invention is by phage display. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to TGF-beta binding protein or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting-Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λImmunoZap™ (H) and λImmunoZap™ (L) vectors (Stratagene, La Jolla, Calif.). Briefly, mRNA is isolated from a B-cell population, and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers, and these genes can be used to generate the bispecific binding molecules of the invention. These primers may be synthesized by one of ordinary skill in the art, or may be purchased from commercially available sources. (See, e.g., Stratagene (La Jolla, Calif.), which sells primers for mouse and human variable regions including, among others, primers for VHa, VHb, VHc, VHd, CH1, VL and CL regions.) These primers may be used to amplify heavy or light chain variable regions, which may then be inserted into vectors such as ImmunoZAP™ H or ImmunoZAP™ L (Stratagene), respectively. These vectors may then be introduced into E. coli, yeast, or mammalian-based systems for expression. Large amounts of a single-chain protein containing a fusion of the VH and VL domains may be produced using these methods (see Bird et al., Science 242:423-426, 1988).

In certain embodiments, the antibodies to be used to generate the bispecific binding molecules of the invention are obtained from transgenic animals (e.g., mice) that produce “heavy chain only” antibodies or “HCAbs.” HCAbs are analogous to naturally occurring camel and llama single-chain VHH antibodies. See, for example, U.S. Pat. Nos. 8,507,748 and 8,502,014, and U.S. Patent Application Publication Nos. US2009/0285805A1, US2009/0169548A1, US2009/0307787A1, US2011/0314563A1, US2012/0151610A1, W02008/122886A2, and W02009/013620A2.

Once cells producing molecules according to the invention have been obtained using any of the above-described immunization and other techniques, the specific antibody genes may be cloned by isolating and amplifying DNA or mRNA therefrom according to standard procedures as described herein and then used to generate the bispecific binding constructs of the present invention. The antibodies produced therefrom may be sequenced and the CDRs identified and the DNA coding for the CDRs may be manipulated as described previously to generate other bispecific binding constructs according to the invention.

Molecular evolution of the complementarity determining regions (CDRs) in the center of the antibody binding site also has been used to isolate antibodies with increased affinity, for example, those as described by Schier et al., 1996, J. Mol. Biol. 263:551. Accordingly, such techniques are useful in preparing binding constructs of the invention.

Although human, partially human, or humanized antibodies will be suitable for many applications, particularly those of the present invention, other types of bispecific binding constructs will be suitable for certain applications. These non-human antibodies can be, for example, derived from any antibody-producing animal, such as mouse, rat, rabbit, goat, donkey, or non-human primate (for example, monkey such as cynomologous or rhesus monkey) or ape (e.g., chimpanzee)). An antibody from a particular species can be made by, for example, immunizing an animal of that species with the desired immunogen or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species. Then, the desired binding region sequences can be used to generate the bispecific binding constructs of the present invention.

Where it is desired to improve the affinity of binding constructs according to the invention containing one or more of the above-mentioned CDRs can be obtained by a number of affinity maturation protocols including maintaining the CDRs (Yang et al., J. Mol. Biol., 254, 392-403, 1995), chain shuffling (Marks et al., Bio/Technology, 10, 779-783, 1992), use of mutation strains of E. coli. (Low et al., J. Mol. Biol., 250, 350-368, 1996), DNA shuffling (Patten et al., Curr. Opin. Biotechnol., 8, 724-733, 1997), phage display (Thompson et al., J. Mol. Biol., 256, 7-88, 1996) and additional PCR techniques (Crameri, et al., Nature, 391, 288-291, 1998). All of these methods of affinity maturation are discussed by Vaughan et al. (Nature Biotechnology, 16, 535-539, 1998).

In certain embodiments, to generate the HHLL bispecific binding constructs of the present invention it may first be desirable to generate a more typical single chain antibody which may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87. These single chain antibodies are distinct from and differ from the bispecific binding constructs of the invention.

Antigen binding fragments derived from an antibody can also be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)2. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

In certain embodiments, the bispecific binding constructs comprise one or more complementarity determining regions (CDRs) of an antibody. CDRs can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). The antibody fragment further may comprise at least one variable region domain of an antibody described herein. Thus, for example, the V region domain may be monomeric and be a VH or VL domain, which is capable of independently binding a desired target (e.g., human CD3) with an affinity at least equal to 10-7M or less as described herein.

The variable region may be any naturally occurring variable domain or an engineered version thereof. By engineered version is meant a variable region that has been created using recombinant DNA engineering techniques. Such engineered versions include those created, for example, from a specific antibody variable region by insertions, deletions, or changes in or to the amino acid sequences of the specific antibody. One of ordinary skill in the art can use any known methods for identifying amino acid residues appropriate for engineering. Additional examples include engineered variable regions containing at least one CDR and optionally one or more framework amino acids from a first antibody and the remainder of the variable region domain from a second antibody. Engineered versions of antibody variable domains may be generated by any number of techniques with which those having ordinary skill in the art will be familiar. Once these domains are generated, they can further be used to generate the bispecific binding molecules of the invention

The variable region may be covalently attached at a C-terminal amino acid to at least one other antibody domain or a fragment thereof. Thus, for example, a VH that is present in the variable region may be linked to an immunoglobulin CH1 domain. Similarly a VL domain may be linked to a CK domain. In this way, for example, the construct may be a Fab fragment wherein the antigen binding domain contains associated VH and VL domains covalently linked at their C-termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

### Binding Specificity

An antibody or a bispecific binding construct “specifically binds” to an antigen if it binds to the antigen with a tight binding affinity as determined by an equilibrium dissociation constant (KD, or corresponding KD, as defined below) value of 10-7 M or less.

Affinity can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet® analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody. These assays can be readily adapted to the bispecific binding constructs of the invention.

Further embodiments of the invention provide bispecific binding constructs that bind to desired targets with an equilibrium dissociation constant or KD (koff/kon) of less than 10-7 M, or of less than 10-8 M, or of less than 10-9 M, or of less than 10-10 M, or of less than 10-11 M, or of less than 10-12 M, or of less than 10-13 M, or of less than 5×10-13 M (lower values indicating tighter binding affinity). Yet further embodiments of the invention are bispecific binding constructs that bind to desired targets with an with an equilibrium dissociation constant or KD (koff/kon) of less than about 10-7 M, or of less than about 10-8 M, or of less than about 10-9 M, or of less than about 10-10 M, or of less than about 10-11 M, or of less than about 10-12 M, or of less than about 10-13 M, or of less than about 5×10-13 M.

In still another embodiment, bispecific binding constructs that bind to desired targets have an equilibrium dissociation constant or KD (koff/kon) of between about 10-7 M and about 10-8 M, between about 10-8 M and about 10-9 M, between about 10-9 M and about 10-10 M, between about 10-10 M and about 10-11 M, between about 10-11 M and about 10-12 M, between about 10-12 M and about 10-13 M. In still another embodiment, a binding construct of the invention have an equilibrium dissociation constant or KD (koff/kon) of between 10-7 M and 10-8 M, between 10-8 M and 10-9 M, between 10-9 M and 10-10 M, between 10-10 M and 10-11 M, between 10-11 M and 10-12 M, between 10-12 M and 10-13 M.

### Molecule Stability

Various aspects of molecule stability may be desired, particularly in the context of a biopharmaceutical therapeutic molecule. For example, stability at various temperatures (“thermostability”) may be desired. In some embodiments, this can encompass stability at physiologic temperature ranges, e.g., at or about 37° C., or from 32° C. to 42° C. In other embodiments, this can encompass stability at higher temperature ranges, e.g., 42° C. to 60° C. In other embodiments, this can encompass stability at cooler temperature ranges, e.g. 20° C. to 32° C. In yet other embodiments, this can encompass stability while in the frozen state, e.g. 0° C. or lower.

Assays to determine thermostability of protein molecules are known in the art. For example, the fully automated UNcle platform (Unchained Labs) which allowed for simultaneous acquisition of intrinsic protein fluorescence and static light scattering (SLS) data during thermal ramp was used and is further described in the Examples. Additionally, thermal stability and aggregation assays described herein in the Examples, such as differential scanning fluorimetry (DSF) and static light scattering (SLS), can also be used to measure both thermal melting (Tm) and thermal aggregation (Tagg) respectively.

Alternatively, accelerated stress studies can be performed on the molecules. Briefly, this involves incubating the protein molecules at a particular temperature (e.g., 40° C.) and then measuring aggregation by size exclusion chromatography (SEC) at various timepoints, where lower levels of aggregation indicate better protein stability.

Alternatively, the thermostability parameter can be determined in terms of molecule aggregation temperature as follows: Molecule solution at a concentration 250 μg/ml is transferred into a single use cuvette and placed in a Dynamic Light Scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the molecule.

Alternatively, temperature melting curves can be determined by Differential Scanning calorimetry (DSC) to determine intrinsic biophysical protein stabilities of the binding constructs. These experiments are performed using a MicroCal LLC (Northampton, Mass., U.S.A) VP-DSC device. The energy uptake of a sample containing a binding construct is recorded from 20° C. to 90° C. compared to a sample containing only the formulation buffer. The binding constructs are adjusted to a final concentration of 250 μg/ml e.g. in SEC running buffer. For recording of the respective melting curve, the overall sample temperature is increased stepwise. At each temperature T energy uptake of the sample and the formulation buffer reference is recorded. The difference in energy uptake Cp (kcal/mole/° C.) of the sample minus the reference is plotted against the respective temperature. The melting temperature is defined as the temperature at the first maximum of energy uptake.

In a further embodiment the bispecific binding constructs according to the invention is stable at or about physiologic pH, i.e., about pH 7.4. In other embodiments, the bispecific binding constructs are stable at a lower pH, e.g., down to pH 6.0. In other embodiments, the bispecific binding constructs are stable at a higher pH, e.g., up to pH 9.0. In one embodiment, the bispecific binding constructs are stable at a pH of 6.0 to 9.0. In another embodiment, the bispecific binding constructs are stable at a pH of 6.0 to 8.0. In another embodiment, the bispecific binding constructs are stable at a pH of 7.0 to 9.0.

In certain embodiments, the more tolerant the bispecific binding construct is to unphysiologic pH (e.g., pH 6.0), the higher the recovery of the binding construct eluted from an ion exchange column is relative to the total amount of loaded protein. In one embodiment, recovery of the binding construct from an ion (e.g., cation) exchange column is ≥30%. In another embodiment, recovery of the binding construct from an ion (e.g., cation) exchange column is ≥40%. In another embodiment, recovery of the binding construct from an ion (e.g., cation) exchange column is ≥50%. In another embodiment, recovery of the binding construct from an ion (e.g., cation) exchange column is ≥60%. In another embodiment, recovery of the binding construct from an ion (e.g., cation) exchange column is ≥70%. In another embodiment, recovery of the binding construct from an ion (e.g., cation) exchange column is ≥80%. In another embodiment, recovery of the binding construct from an ion (e.g., cation) exchange column is ≥90%. In another embodiment, recovery of the binding construct from an ion (e.g., cation) exchange column is ≥95%. In another embodiment, recovery of the binding construct from an ion (e.g., cation) exchange column is ≥99%.

In certain embodiments, it may be desired to determine the chemical stability of the molecules. Determination of bispecific binding construct chemical stability can be carried out via isothermal chemical denaturation (“ICD”) by monitoring intrinsic protein fluorescence, as further described herein in the Examples. ICD yields C1/2 and ΔG which can be good metrics for protein stability. C1/2 is the amount of chemical denaturant required to denature 50% of the protein and is used to derive ΔG (or unfolding energy).

Clipping of protein chains is another critical product quality attribute that is carefully monitored and reported for biologic drugs. Typically, a longer and/or a less structured linker is expected to result in increased clipping as a function of incubation time and temperature. Clipping is a critical issue for bispecific binding constructs as clips to linkers connecting either the target or T-cell engaging domains have terminal detrimental impact on drug potency and efficacy. Clips to additional sites including the scFc may impact pharmaco-dynamic/kinetic properties. Increased clipping is an attribute to be avoided in a pharmaceutical product. Accordingly, in certain embodiments, protein clipping can be assayed as described herein in the Examples.

### Immune Effector Cells and Effector Cell Proteins

A bispecific binding construct can bind to a molecule expressed on the surface of an immune effector cell (called “effector cell protein” herein) and to another molecule expressed on the surface of a target cell (called a “target cell protein” herein). The immune effector cell can be a T cell, an NK cell, a macrophage, or a neutrophil. In some embodiments the effector cell protein is a protein included in the T cell receptor (TCR)-CD3 complex. The TCR-CD3 complex is a heteromultimer comprising a heterodimer comprising TCRα and TCRβ or TCRγ and TCRδ plus various CD3 chains from among the CD3 zeta (CD3ζ) chain, CD3 epsilon (CD3ϵ) chain, CD3 gamma (CD3γ) chain, and CD3 delta (CD3δ) chain.

The CD3 receptor complex is a protein complex and is composed of four chains. In mammals, the complex contains a CD3γ (gamma) chain, a CD3δ (delta) chain, and two CD3ε (epsilon) chains. These chains associate with the T cell receptor (TCR) and the so-called ζ (zeta) chain to form the T cell receptor CD3 complex and to generate an activation signal in T lymphocytes. The CD3γ (gamma), CD3δ (delta), and CD3ε(epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. The most preferred epitope of CD3 epsilon is comprised within amino acid residues 1-27 of the human CD3 epsilon extracellular domain. It is envisaged that the bispecific binding constructs according to the present invention typically and advantageously show less unspecific T cell activation, which is not desired in specific immunotherapy. This translates to a reduced risk of side effects.

In some embodiments the effector cell protein can be the human CD3 epsilon (CD3ϵ) chain (the mature amino acid sequence of which is disclosed in SEQ ID NO: 40), which can be part of a multimeric protein. Alternatively, the effector cell protein can be human and/or cynomolgus monkey TCRα, TCRβ, TCRδ, TCRγ, CD3 beta (CD3β) chain, CD3 gamma (CD3γ) chain, CD3 delta (CD3δ) chain, or CD3 zeta (CD3ζ) chain.

Moreover, in some embodiments, a bispecific binding construct can also bind to a CD3ϵ chain from a non-human species, such as mouse, rat, rabbit, new world monkey, and/or old world monkey species. Such species include, without limitation, the following mammalian species: Mus musculus; Rattus rattus; Rattus norvegicus; the cynomolgus monkey, Macaca fascicularis; the hamadryas baboon, Papio hamadryas; the Guinea baboon, Papio papio; the olive baboon, Papio anubis; the yellow baboon, Papio cynocephalus; the Chacma baboon, Papio ursinus; Callithrix jacchus; Saguinus Oedipus; and Saimiri sciureus. The mature amino acid sequence of the CD3ϵ chain of cynomolgus monkey is provided in SEQ ID NO: 41. Having a therapeutic molecule that has comparable activity in humans and species commonly used for preclinical testing, such as mice and monkeys, can simplify, accelerate, and ultimately provide improved outcomes in drug development. In the long and expensive process of bringing a drug to market, such advantages can be critical.

In certain embodiments, the bispecific binding construct can bind to an epitope within the first 27 amino acids of the CD3ϵ chain (SEQ ID NO: 43), which may be a human CD3ϵ chain or a CD3ϵ chain from different species, particularly one of the mammalian species listed herein. The epitope can contain the amino acid sequence Gln-Asp-Gly-Asn-Glu (SEQ ID NO; 104). The advantages of a binding construct that binds such an epitope are explained in detail in U.S. Patent Application Publication 2010/0183615A1, the relevant portions of which are incorporated herein by reference. The epitope to which an antibody or bispecific binding construct binds can be determined by alanine scanning, which is described in, e.g., U.S. Patent Application Publication 2010/0183615A1, the relevant portions of which are incorporated herein by reference. In other embodiments, the bispecific binding construct can bind to an epitope within the extracellular domain of CD3ϵ (SEQ ID NO: 42).

In embodiments where a T cell is the immune effector cell, effector cell proteins to which a bispecific binding construct can bind include, without limitation, the CD3ϵ chain, the CD3γ, the CD3δ chain, the CD3ζ chain, TCRα, TCRβ, TCRγ, and TCRδ. In embodiments where an NK cell or a cytotoxic T cell is an immune effector cell, NKG2D, CD352, NKp46, or CD16a can, for example, be an effector cell protein. In embodiments where a CD8+ T cell is an immune effector cell, 4-1BB or NKG2D, for example, can be an effector cell protein. Alternatively, in other embodiments a bispecific binding construct could bind to other effector cell proteins expressed on T cells, NK cells, macrophages, or neutrophils.

### Target Cells and Target Cell Proteins Expressed on Target Cells

As explained herein, a bispecific binding construct can bind to an effector cell protein and a target cell protein. The target cell protein can, for example, be expressed on the surface of a cancer cell, a cell infected with a pathogen, or a cell that mediates a disease, for example an inflammatory, autoimmune, and/or fibrotic condition. In some embodiments, the target cell protein can be highly expressed on the target cell, although high levels of expression are not necessarily required.

Where the target cell is a cancer cell, a bispecific binding construct as described herein can bind to a cancer cell antigen as described herein. A cancer cell antigen can be a human protein or a protein from another species. For example, a bispecific binding construct may bind to a target cell protein from a mouse, rat, rabbit, new world monkey, and/or old world monkey species, among many others. Such species include, without limitation, the following species: Mus musculus; Rattus rattus; Rattus norvegicus; cynomolgus monkey, Macaca fascicularis; the hamadryas baboon, Papio hamadryas; the Guinea baboon, Papio papio; the olive baboon, Papio anubis; the yellow baboon, Papio cynocephalus; the Chacma baboon, Papio ursinus, Callithrix jacchus, Saguinus oedipus, and Saimiri sciureus.

In some examples, the target cell protein can be a protein selectively expressed on an infected cell. For example, in the case of an HBV or HCV infection, the target cell protein can be an envelope protein of HBV or HCV that is expressed on the surface of an infected cell. In other embodiments, the target cell protein can be gp120 encoded by human immunodeficiency virus (HIV) on HIV-infected cells.

In other aspects, a target cell can be a cell that mediates an autoimmune or inflammatory disease. For example, human eosinophils in asthma can be target cells, in which case, EGF-like module containing mucin-like hormone receptor (EMR1), for example, can be a target cell protein. Alternatively, excess human B cells in a systemic lupus erythematosus patient can be target cells, in which case CD19 or CD20, for example, can be a target cell protein. In other autoimmune conditions, excess human Th2 T cells can be target cells, in which case CCR4 can, for example, be a target cell protein. Similarly, a target cell can be a fibrotic cell that mediates a disease such as atherosclerosis, chronic obstructive pulmonary disease (COPD), cirrhosis, scleroderma, kidney transplant fibrosis, kidney allograft nephropathy, or a pulmonary fibrosis, including idiopathic pulmonary fibrosis and/or idiotypic pulmonary hypertension. For such fibrotic conditions, fibroblast activation protein alpha (FAP alpha) can, for example, be a target cell protein.

### Therapeutic Methods and Compositions

Bispecific binding constructs can be used to treat a wide variety of conditions including, for example, various forms of cancer, infections, autoimmune or inflammatory conditions, and/or fibrotic conditions.

Another embodiment provides the use of the binding construct of the invention (or of the binding construct produced according to the process of the invention) in the manufacture of a medicament for the prevention, treatment or amelioration of a disease.

Provided herein are pharmaceutical compositions comprising bispecific binding constructs. These pharmaceutical compositions comprise a therapeutically effective amount of a bispecific binding construct and one or more additional components such as a physiologically acceptable carrier, excipient, or diluent. In some embodiments, these additional components can include buffers, carbohydrates, polyols, amino acids, chelating agents, stabilizers, and/or preservatives, among many possibilities.

In some embodiments, a bispecific binding construct can be used to treat cell proliferative diseases, including cancer, which involve the unregulated and/or inappropriate proliferation of cells, sometimes accompanied by destruction of adjacent tissue and growth of new blood vessels, which can allow invasion of cancer cells into new areas, i.e. metastasis. Included within conditions treatable with a bispecific binding construct are non-malignant conditions that involve inappropriate cell growth, including colorectal polyps, cerebral ischemia, gross cystic disease, polycystic kidney disease, benign prostatic hyperplasia, and endometriosis. A bispecific binding construct can be used to treat a hematologic or solid tumor malignancy. More specifically, cell proliferative diseases that can be treated using a bispecific binding construct are, for example, cancers including mesotheliomas, squamous cell carcinomas, myelomas, osteosarcomas, glioblastomas, gliomas, carcinomas, adenocarcinomas, melanomas, sarcomas, acute and chronic leukemias, lymphomas, and meningiomas, Hodgkin's disease, Sézary syndrome, multiple myeloma, and lung, non-small cell lung, small cell lung, laryngeal, breast, head and neck, bladder, ovarian, skin, prostate, cervical, vaginal, gastric, renal cell, kidney, pancreatic, colorectal, endometrial, and esophageal, hepatobiliary, bone, skin, and hematologic cancers, as well as cancers of the nasal cavity and paranasal sinuses, the nasopharynx, the oral cavity, the oropharynx, the larynx, the hypolarynx, the salivary glands, the mediastinum, the stomach, the small intestine, the colon, the rectum and anal region, the ureter, the urethra, the penis, the testis, the vulva, the endocrine system, the central nervous system, and plasma cells.

Among the texts providing guidance for cancer therapy is Cancer, Principles and Practice of Oncology, 4th Edition, DeVita et al., Eds. J. B. Lippincott Co., Philadelphia, Pa. (1993). An appropriate therapeutic approach is chosen according to the particular type of cancer, and other factors such as the general condition of the patient, as is recognized in the pertinent field. A bispecific binding construct can be added to a therapy regimen using other anti-neoplastic agents in treating a cancer patient.

In some embodiments, a bispecific binding construct can be administered concurrently with, before, or after a variety of drugs and treatments widely employed in cancer treatment such as, for example, chemotherapeutic agents, non-chemotherapeutic, anti-neoplastic agents, and/or radiation. For example, chemotherapy and/or radiation can occur before, during, and/or after any of the treatments described herein. Examples of chemotherapeutic agents are discussed herein and include, but are not limited to, cisplatin, taxol, etoposide, mitoxantrone (Novantrone®), actinomycin D, cycloheximide, camptothecin (or water soluble derivatives thereof), methotrexate, mitomycin (e.g., mitomycin C), dacarbazine (DTIC), anti-neoplastic antibiotics such as adriamycin (doxorubicin) and daunomycin, and all the chemotherapeutic agents mentioned herein.

A bispecific binding construct can also be used to treat infectious disease, for example a chronic hepatis B virus (HBV) infection, a hepatis C virus (HCV) infection, a human immunodeficiency virus (HIV) infection, an Epstein-Barr virus (EBV) infection, or a cytomegalovirus (CMV) infection, among many others.

A bispecific binding construct can find further use in other kinds of conditions where it is beneficial to deplete certain cell types. For example, depletion of human eosinophils in asthma, excess human B cells in systemic lupus erythematosus, excess human Th2 T cells in autoimmune conditions, or pathogen-infected cells in infectious diseases can be beneficial. In a fibrotic condition, it can be useful to deplete cells forming fibrotic tissue.

Therapeutically effective doses of a bispecific binding construct can be administered. The amount of bispecific binding construct that constitutes a therapeutically dose may vary with the indication treated, the weight of the patient, the calculated skin surface area of the patient. Dosing of a bispecific binding construct can be adjusted to achieve the desired effects. In many cases, repeated dosing may be required.

A bispecific binding construct, or a pharmaceutical composition containing such a molecule, can be administered by any feasible method. Protein therapeutics will ordinarily be administered by a parenteral route, for example by injection, since oral administration, in the absence of some special formulation or circumstance, would lead to hydrolysis of the protein in the acid environment of the stomach. Subcutaneous, intramuscular, intravenous, intraarterial, intralesional, or peritoneal bolus injection are possible routes of administration. A bispecific binding construct can also be administered via infusion, for example intravenous or subcutaneous infusion. Topical administration is also possible, especially for diseases involving the skin. Alternatively, a bispecific binding construct can be administered through contact with a mucus membrane, for example by intra-nasal, sublingual, vaginal, or rectal administration or administration as an inhalant. Alternatively, certain appropriate pharmaceutical compositions comprising a bispecific binding construct can be administered orally.

The term “treatment” encompasses alleviation of at least one symptom or other embodiment of a disorder, or reduction of disease severity, and the like. A bispecific binding construct according to the present invention need not effect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method comprising administering to a patient a bispecific binding construct of the invention in an amount and for a time sufficient to induce a sustained improvement over baseline of an indicator that reflects the severity of the particular disorder.

The term “prevention” encompasses prevention of at least one symptom or other embodiment of a disorder, and the like. A prophylactically administered treatment incorporating a bispecific binding construct according to the present invention need not be completely effective in preventing the onset of a condition in order to constitute a viable prophylactic agent. Simply reducing the likelihood that the disease will occur or worsen in a subject, is sufficient.

As is understood in the pertinent field, pharmaceutical compositions comprising the bispecific binding construct are administered to a subject in a manner appropriate to the indication and the composition. Pharmaceutical compositions may be administered by any suitable technique, including but not limited to parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Delivery by inhalation includes, for example, nasal or oral inhalation, use of a nebulizer, inhalation of the bispecific binding construct in aerosol form, and the like. Other alternatives include oral preparations including pills, syrups, or lozenges.

The bispecific binding constructs can be administered in the form of a composition comprising one or more additional components such as a physiologically acceptable carrier, excipient or diluent. Optionally, the composition additionally comprises one or more physiologically active agents. In various particular embodiments, the composition comprises one, two, three, four, five, or six physiologically active agents in addition to one or more bispecific binding constructs.

Kits for use by medical practitioners are provided including one or more bispecific binding construct and a label or other instructions for use in treating any of the conditions discussed herein. In one embodiment, the kit includes a sterile preparation of one or more bispecific binding constructs which may be in the form of a composition as disclosed herein, and may be in one or more vials.

Dosages and the frequency of administration may vary according to such factors as the route of administration, the particular bispecific binding construct employed, the nature and severity of the disease to be treated, whether the condition is acute or chronic, and the size and general condition of the subject.

Having described the invention in general terms above, the following examples are offered by way of illustration and not limitation.

### EXAMPLES

### Example 1

### Generation and Expression of Bispecific HHLL Binding Constructs with Protease Cleavage Sites

The open reading frames of the different formats (FIG. 1-3) were ordered as gene syntheses and subcloned into a mammalian expression vector containing an IgG derived signal peptide for secreted expression into the cell culture supernatant. Sequence verified plasmid clones were transfected transiently into 293 HEK cells or stably transfected into CHO cells, cell culture supernatant was harvested after 3 days of transient expression or 6 days for stable transfectants. Cell culture supernatant was stored at −80° C. until protein purification.

FIGS. 1-3 show the single chain pro-bispecific binding construct formats (i.e., without protease cleavage) in absence of MMP2/9 and resulting fragments in presence of MMP2/9. Format A contains the following domains from N- to C-terminus: CD3ϵ (a.a. 1-6 or a.a. 1-27) peptide-L0-Anti CD3 VH-L1-Anti MSLN VH-L2-Anti CD3 VL-L3-Anti MSLN VL-L4-HLE domain1-L5-HLE domain2, in which the anti-CD3 and anti-MSLN variable domains contain an engineered disulfide bridge building a covalent bond between the specific VH and VL domains. In this format L0, L1, L3 and L4 contain a MMP2/9 restriction site (SEQ ID NO: 45). Format B contains an N-terminal CD3ϵ (a.a. 1-6 or a.a. 1-27) peptide-L0-Anti CD3 VH-L1-HLE domain1-L2-Anti MSLN VH-L3-Anti CD3 VL-L4-HLE domain2-L5-Anti MSLN VL, in which the anti-CD3 and anti-MSLN variable domains contain an engineered disulfide bridge building a covalent bond between the specific VH and VL domains. In this format L0, L1, L2, L4 and L5 contain a MMP2/9 restriction site. L3 linker length was varied between the constructs V1E (G4S)3, B1U (G4S)6, Z9P (G4S)12. Format C contains the following domains: N-terminal anti CD3 VH-L1-Anti MSLN VH-L2-Anti CD3 VL-L3-Anti MSLN VL-L4-HLE domain1-L5-HLE domain2, in which the anti-CD3 and anti-MSLN variable domains contain an engineered disulfide bridge building a covalent bond between the specific VH and VL domains. In this format L3 contains a MMP2/9 restriction site. Format D contains an N-terminal CD3ε peptide-L0-Human Serum Albumin-L1-anti CD3 VH-L2-Anti MSLN VH-L3-Anti CD3 VL-L4-Anti MSLN VL-L5-HLE domain1-L6-HLE domain2. CD3ε peptide was used in two different lengths (G2P AA1-6, W9A AA1-27), where L0 is an SG linker and L5 is a G4 linker. In this format L1, L2, L4 and L5 contain a MMP2/9 restriction site. A second construct of this format was generated omitting the N-terminal CD3 peptide (O7H). Format E contains an N-terminal CD3 peptide (AA1-6 or AA1-27)-L0-HLE domain1-L1-HLE domain2-L2-anti CD3 VH-L3-anti MSLN VH-L4-anti CD3 VL-L5-anti MSLN VL. In this format L2, L3 and L5 contain a MMP2/9 restriction site. A second construct of this format was generated omitting the N-terminal CD3 peptide (T7U).

### Example 2

### Chromatography Analysis

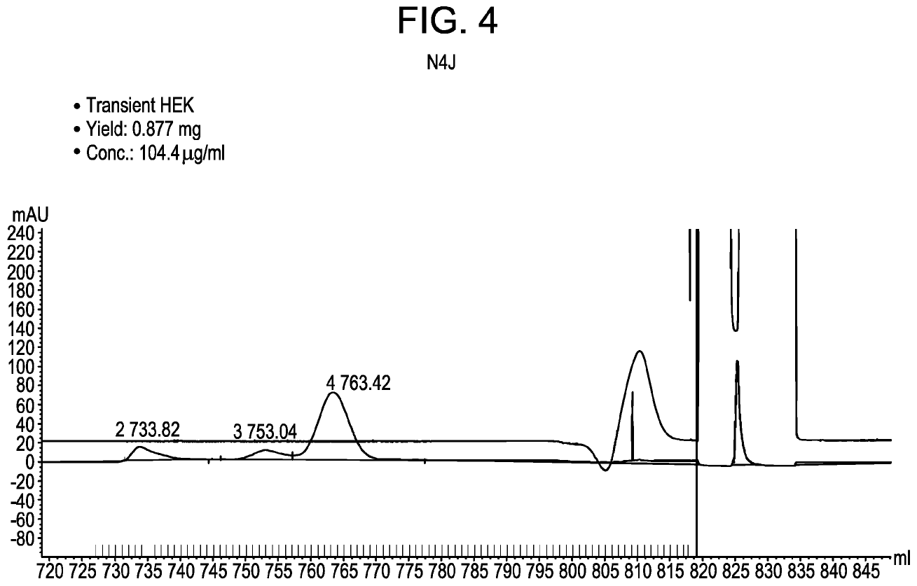

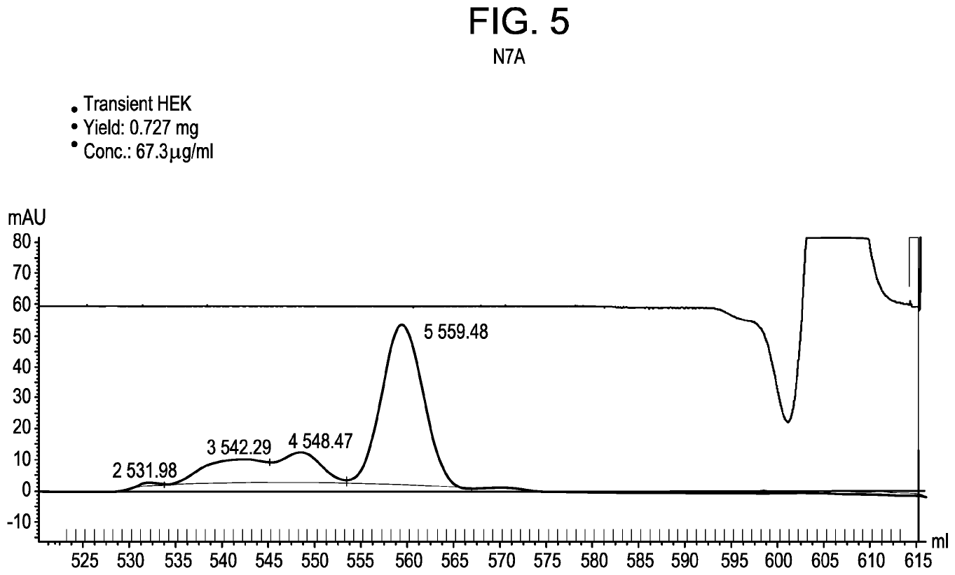

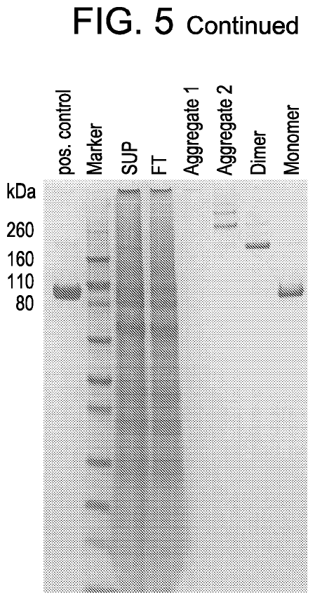

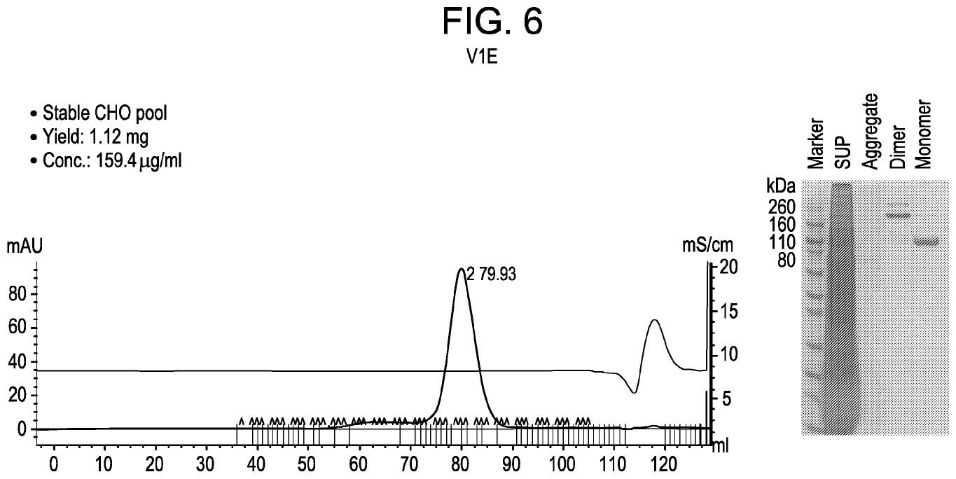

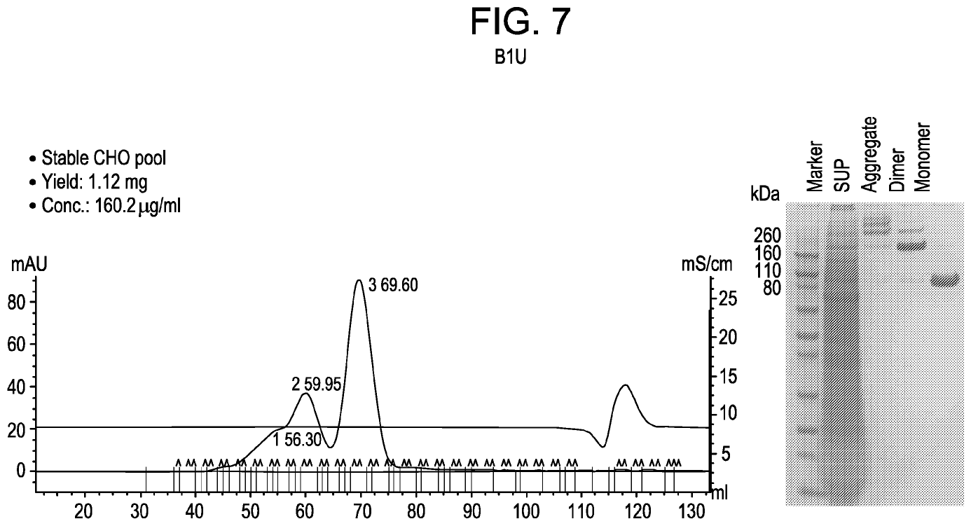

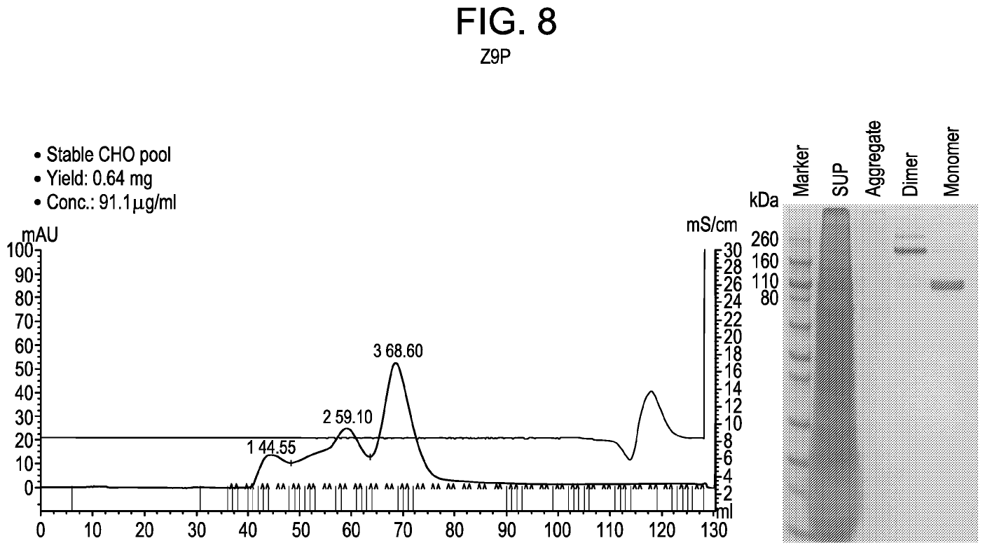

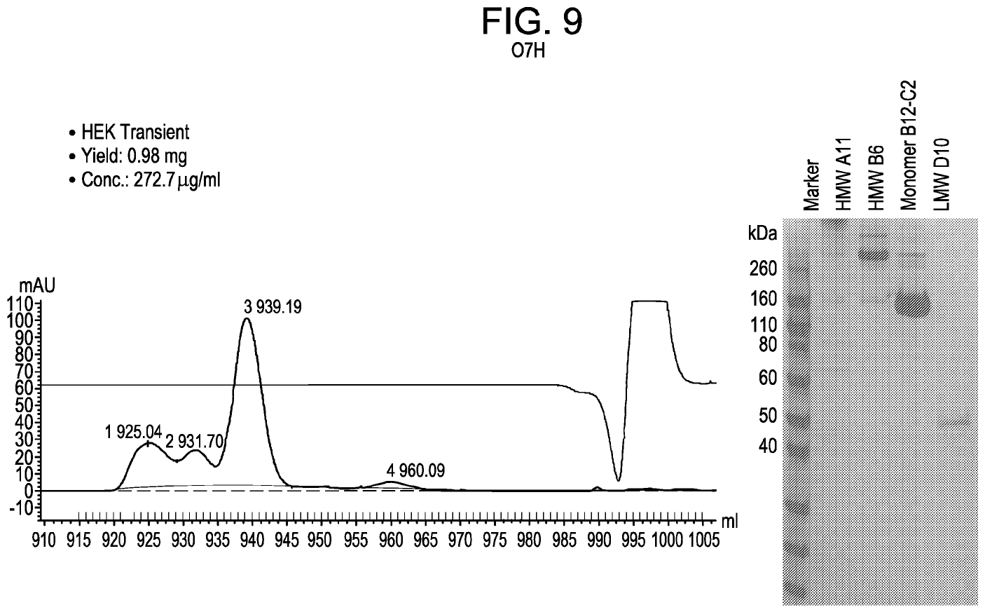

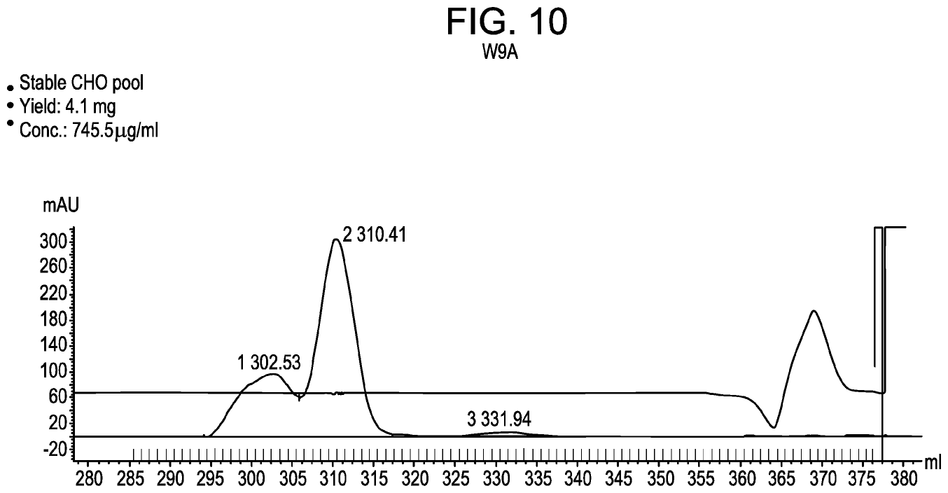

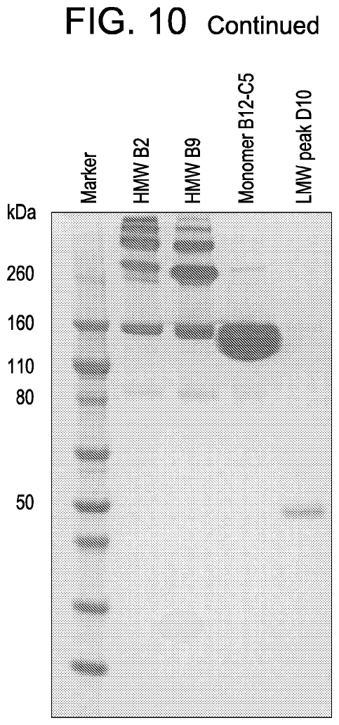

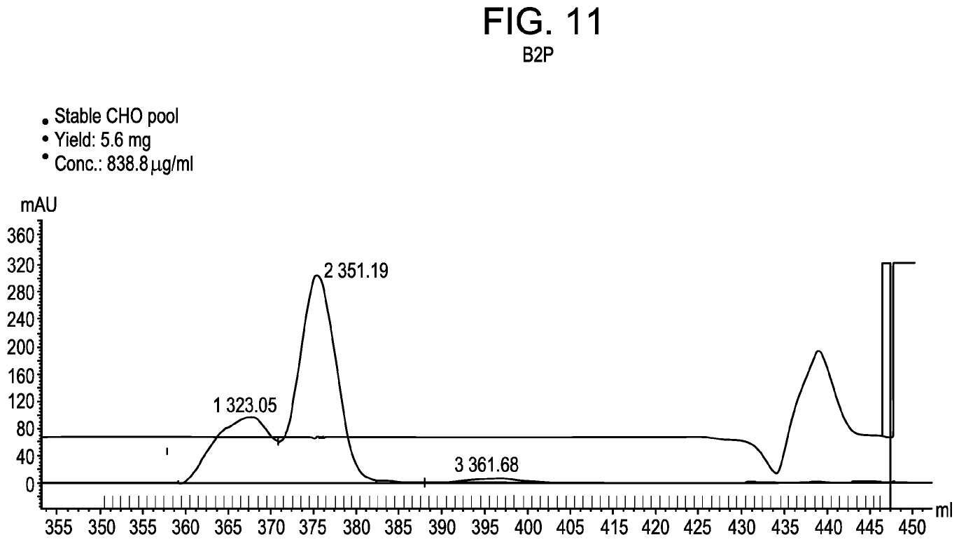

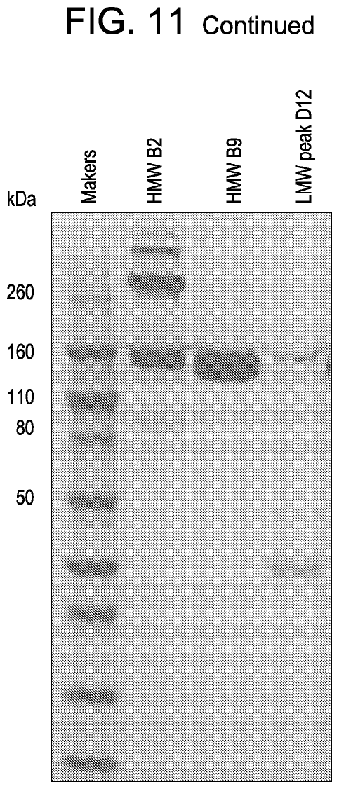

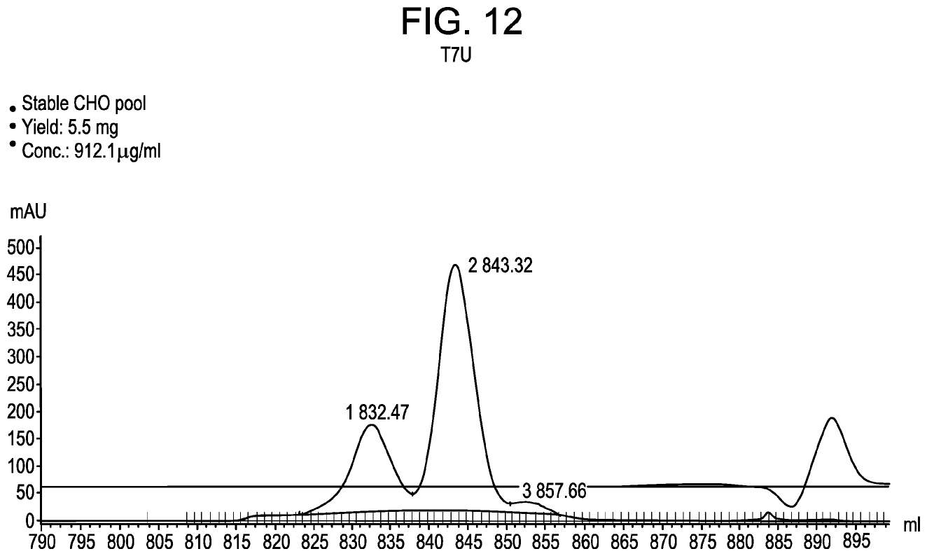

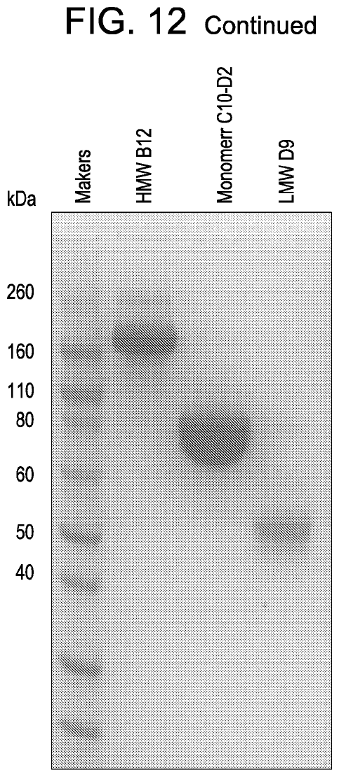

Protein purification was done by Protein A affinity chromatography followed by size exclusion chromatography (Error! Reference source not found. es 3-12). According to the OD280 nm signal (blue) peaks were pooled and MW was analyzed by SDS-PAGE. Protein monomer peaks were formulated in 10 mM Citrate, 75 mM Lysine, 4% Trehalose for aliquoted storage at −80° C. Results for the following constructs are depicted in FIGS. 4-13, respectively: N4J, N7A, V1E, B1U, Z9P, 07H, W9A, B2P, T7U, L2G, indication expression of the various constructs.

### Example 3

### Gel/Blot Size Analysis to Determine if Cleavage Sites are Functional In Vitro

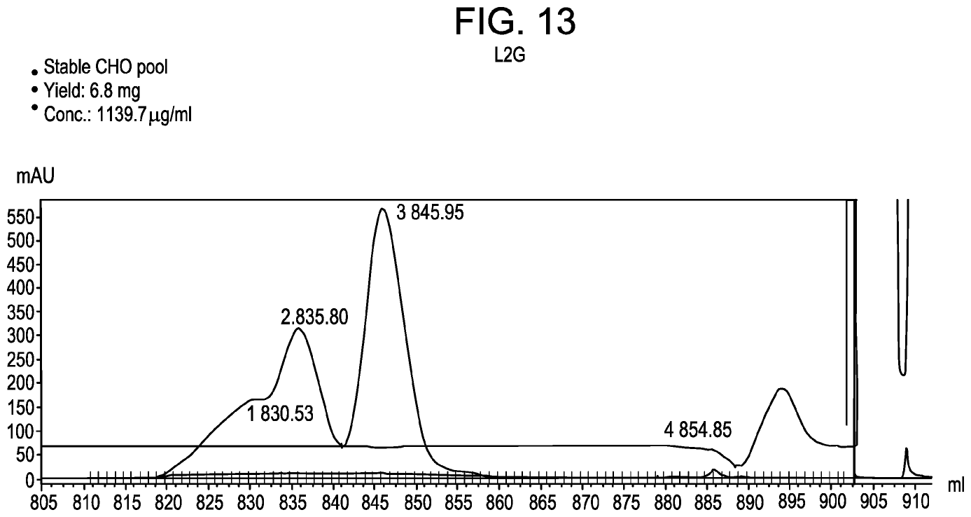

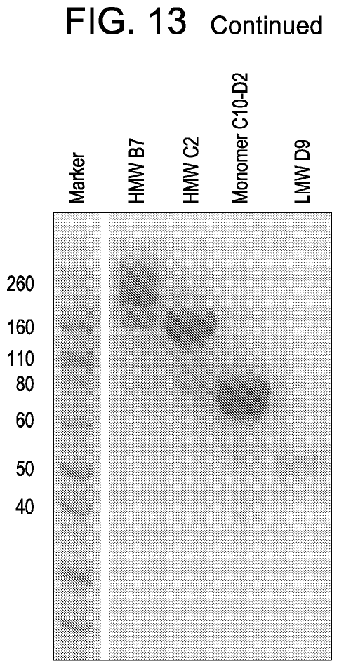

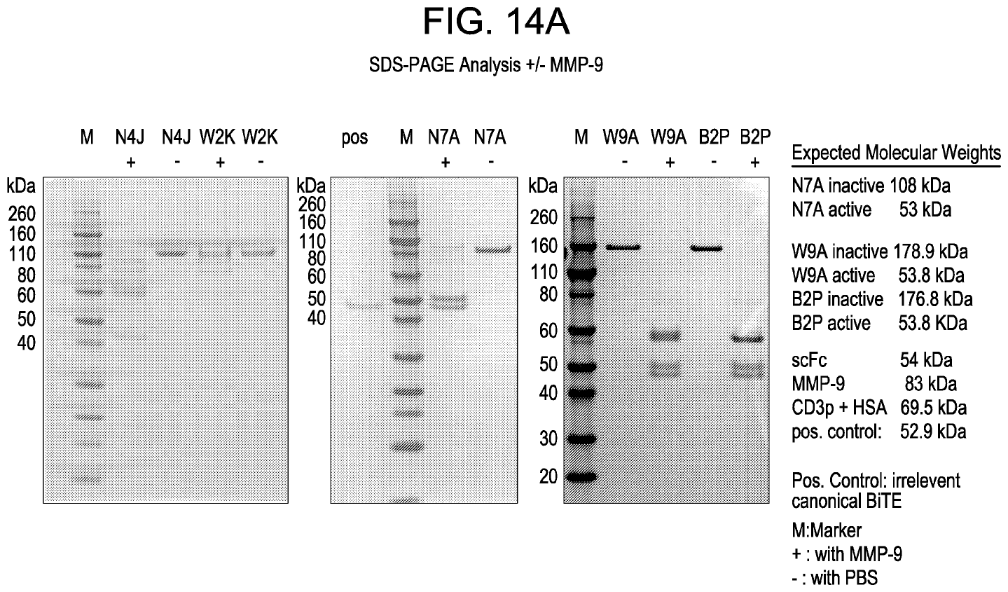

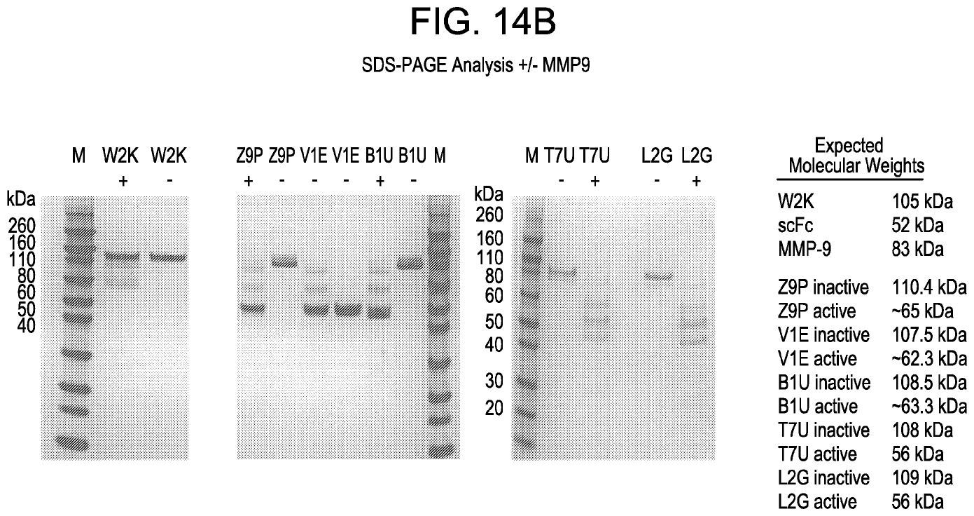

To determine in vitro cleavage of the bispecific binding constructs, purified bispecific binding constructs were incubated with recombinant MMP-9 at a 1:1 molar ratio for 18h at 37° C. (or PBS as a control). Then, samples were denatured by 95° C. for 5 min and applied to non-reducing SDS-PAGE (FIGS. 13-14). The expected MW of the bispecific binding construct in their pro- (i.e., without protease cleavage) conformation in absence of MMP9 (-MMP9) and in their active form (i.e., cleaved by protease) in presence of MMP9 (+MMP9) is shown. Samples incubated with MMP9 were not purified subsequently, indicated by additional MMP9 specific bands (67, 82 kDa). V1E (-MMP9) showed lower MW than expected and no difference to its activated (+MMP9) conformation. Results of this are depicted in FIGS. 14A and 14B.

### Example 4

### In Vitro FACS Binding Analysis

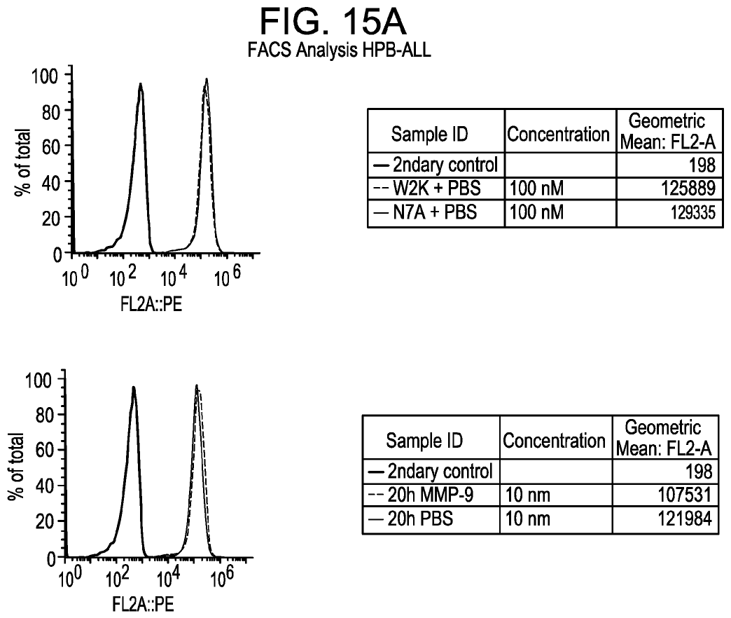

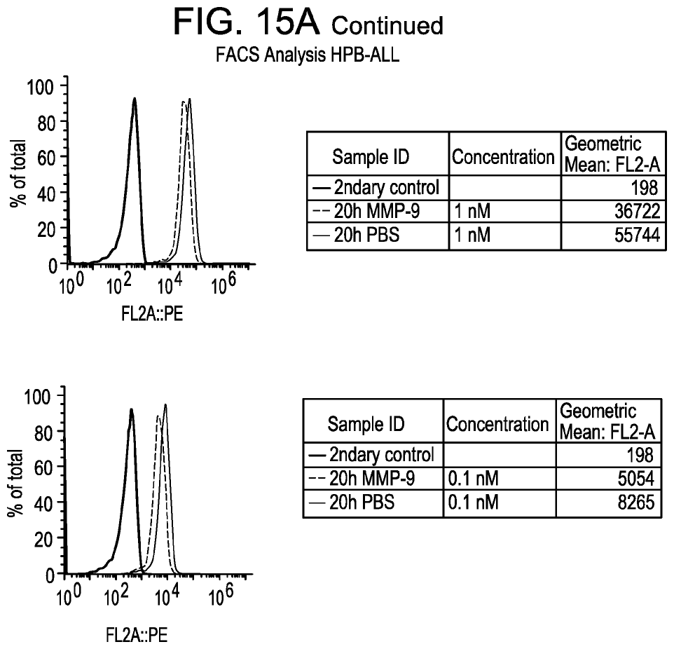

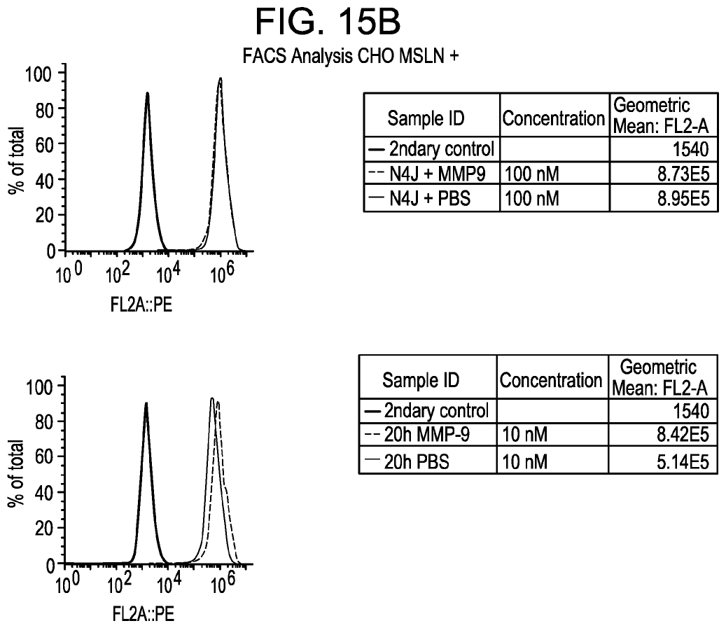

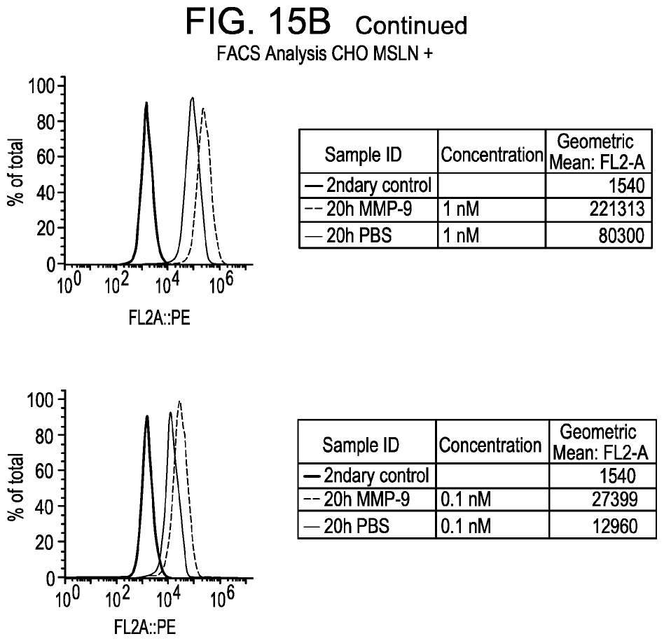

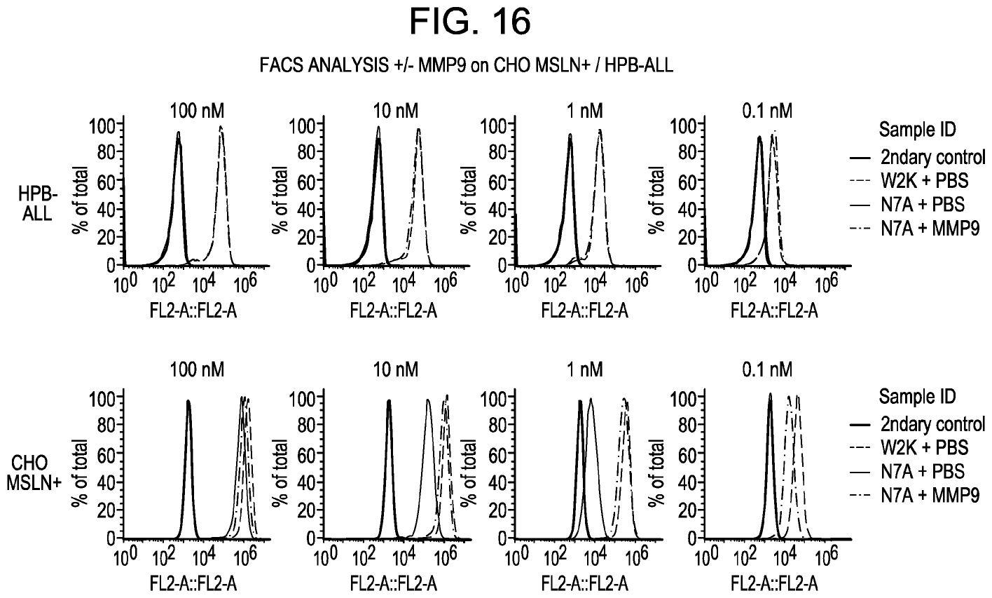

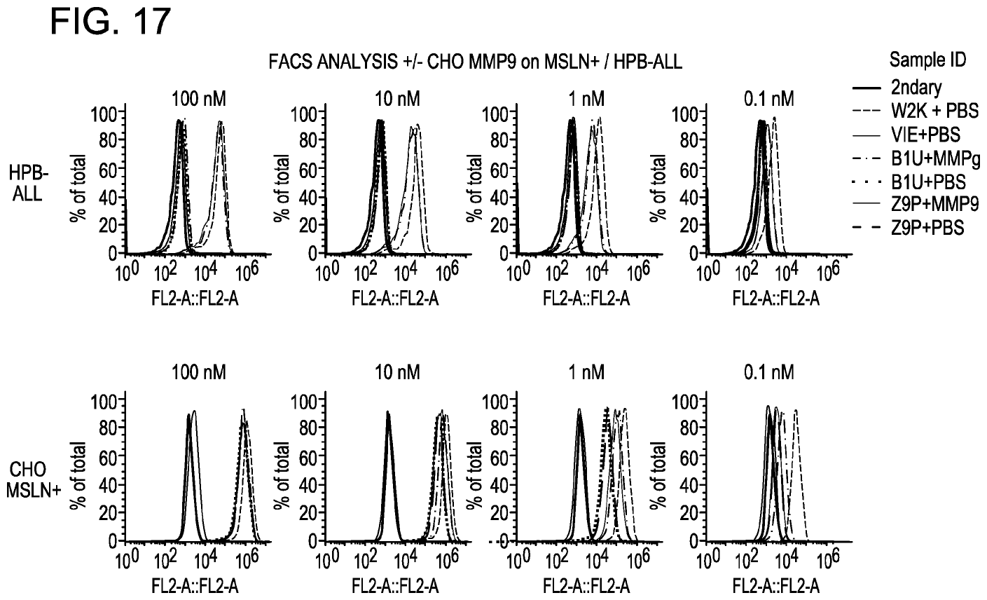

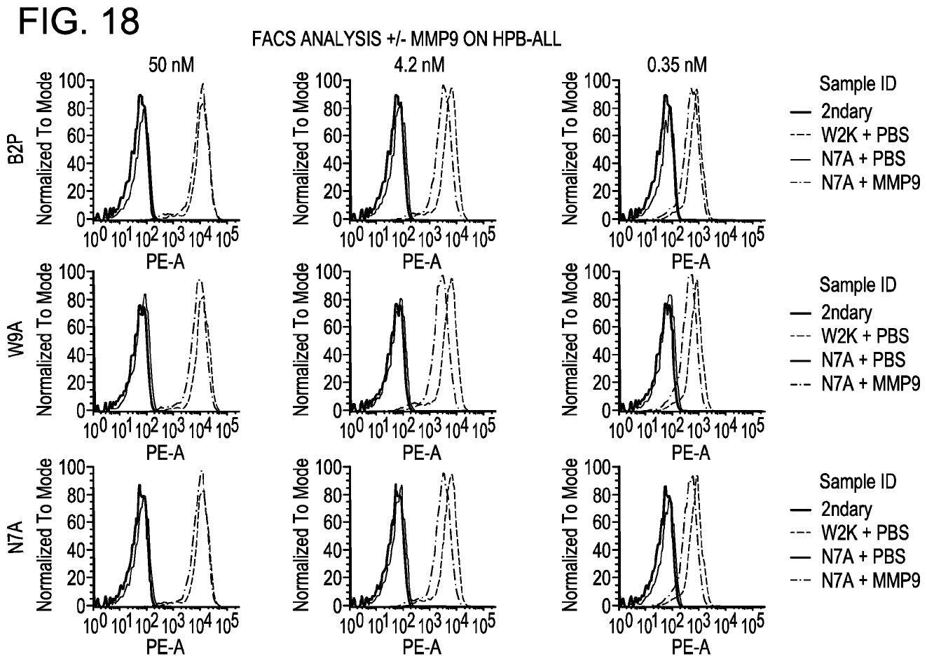

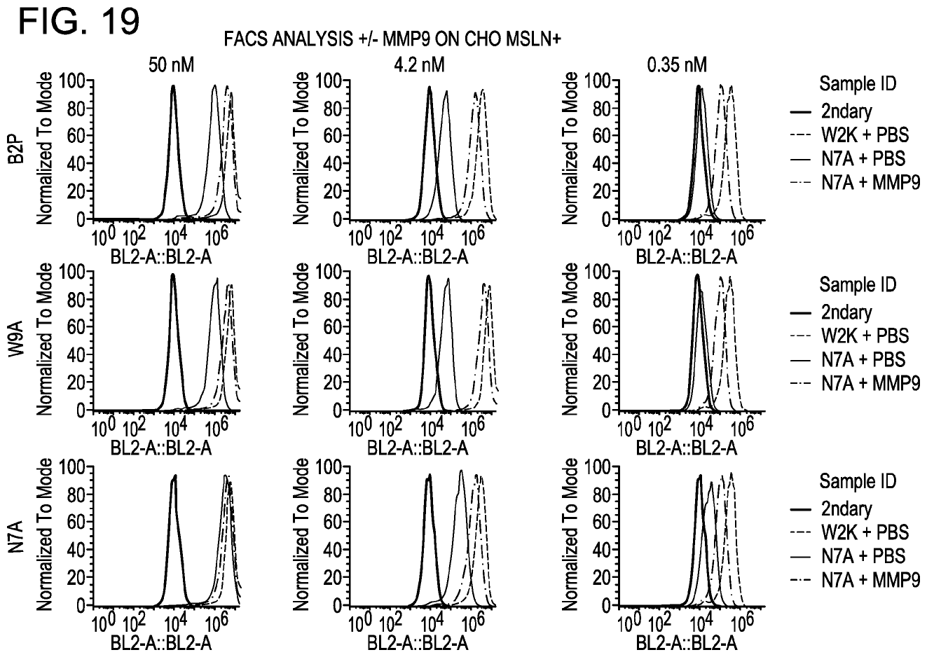

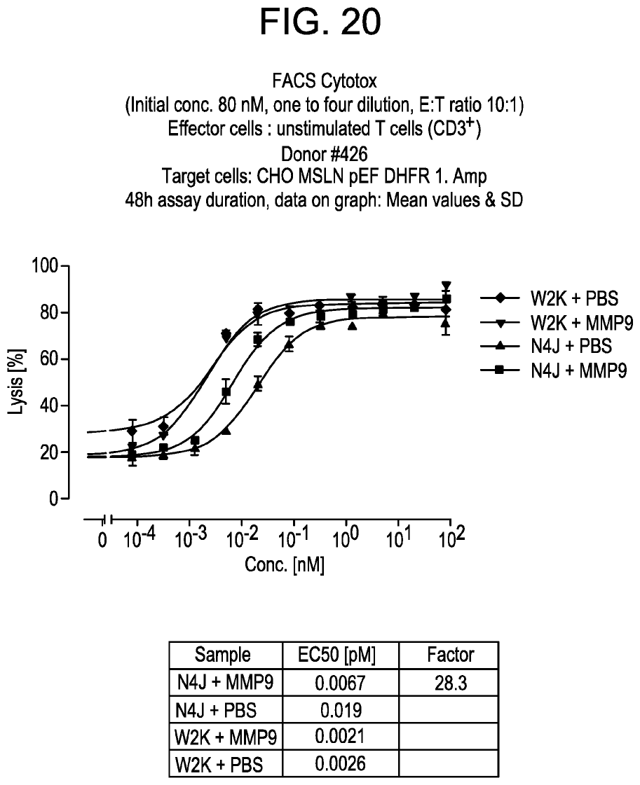

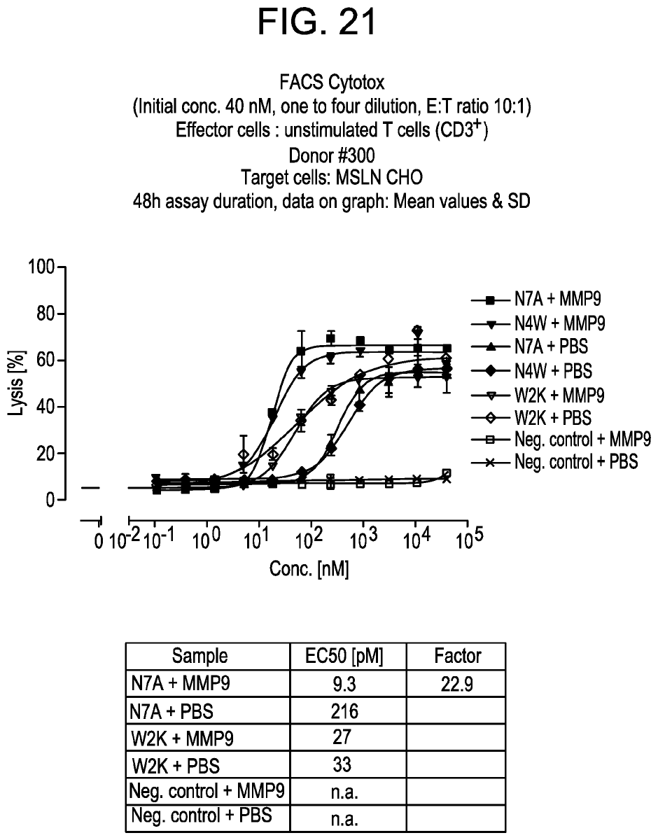

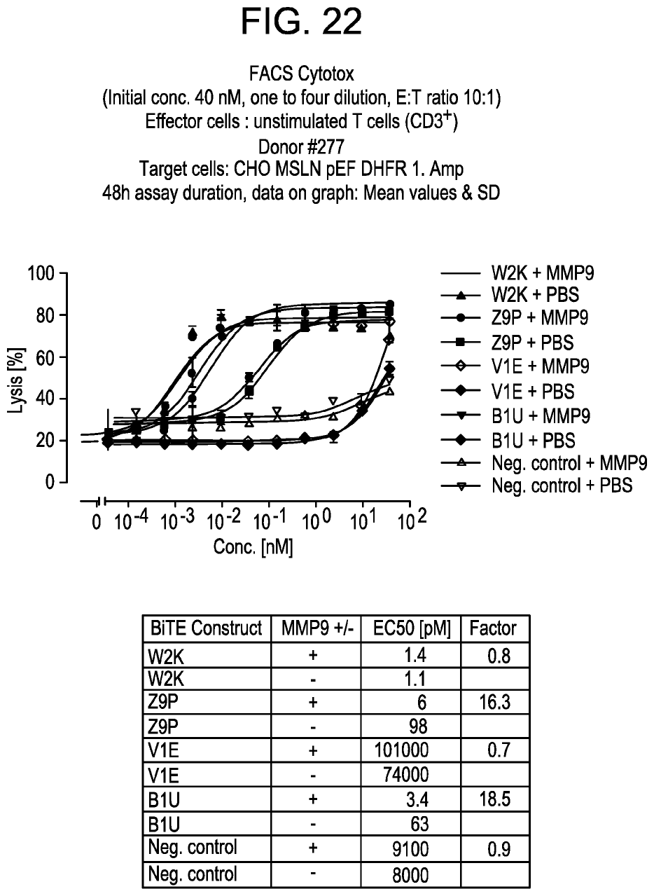

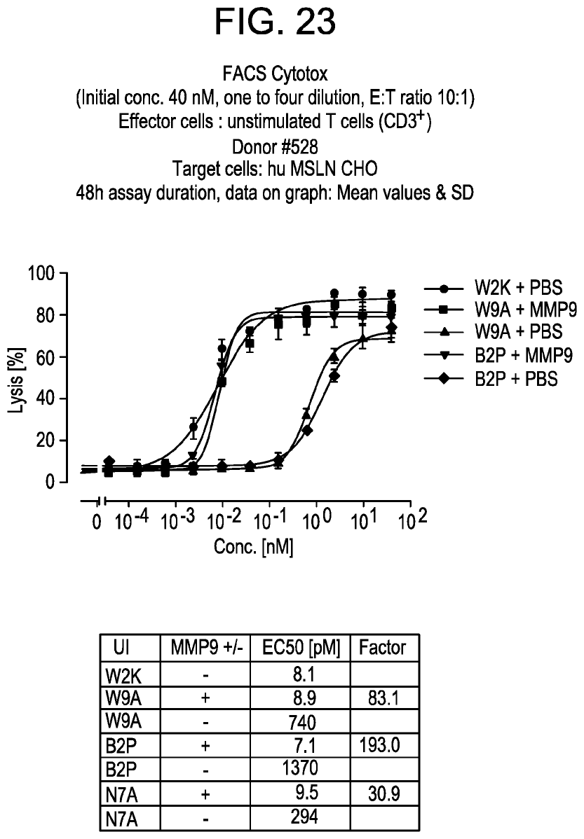

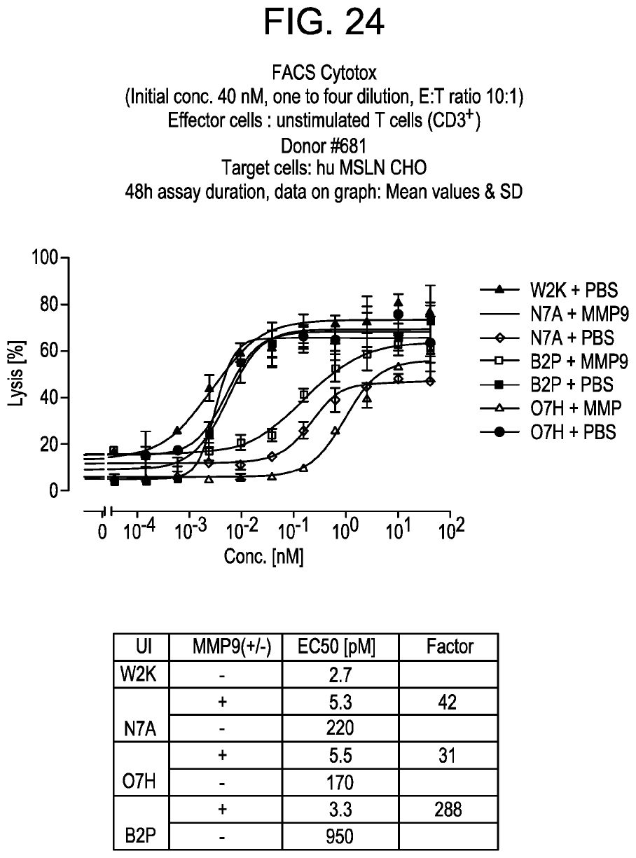

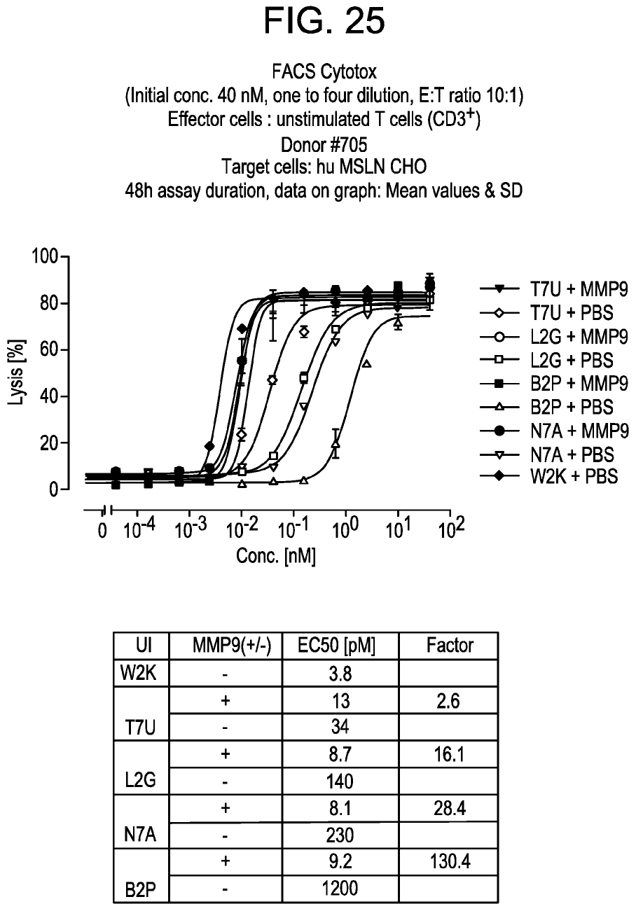

Purified bispecific binding constructs were applied to flow cytometry to determine binding to target antigen transfected CHO cells (MSLN+CHO) or a human CD3 positive T cell line (HPB-ALL). Non-digested and MMP-9 digested bispecific binding constructs and a BiTE®-HLE construct (W2K) were compared for binding signals at both conditions (Figures Error! Reference source not found. 15-19). The N4J bispecific binding construct was pre-incubated at 1:1 molar ratio with huMMP-9 or PBS for 20 h at 37° C. In FIGS. 15-17, bispecific molecules were stained using a 3E5A5 mouse anti-(anti-CD3 scFv) Ab (5 μ/ml) and PE anti mouse IgG (1:200). Assay was run at 100/10/1/0.1 nM bispecific binding constructs for 30 minutes at 4° C. Staining was referenced to cells only stained by the secondary anti-mouse Fc-specific PE-conjugated polyclonal Ab. In FIGS. 18 and 19, the bispecific binding constructs were pre-incubated at a 1:1 molar ratio with huMMP-9 or PBS for 18 hours at 37° C. and the assay was run at 50/4.2/1/0.35 nM bispecific binding constructs for 30 minutes at 4° C.

### Example 5

### FACS-Based In Vitro Cytotoxicity Assays

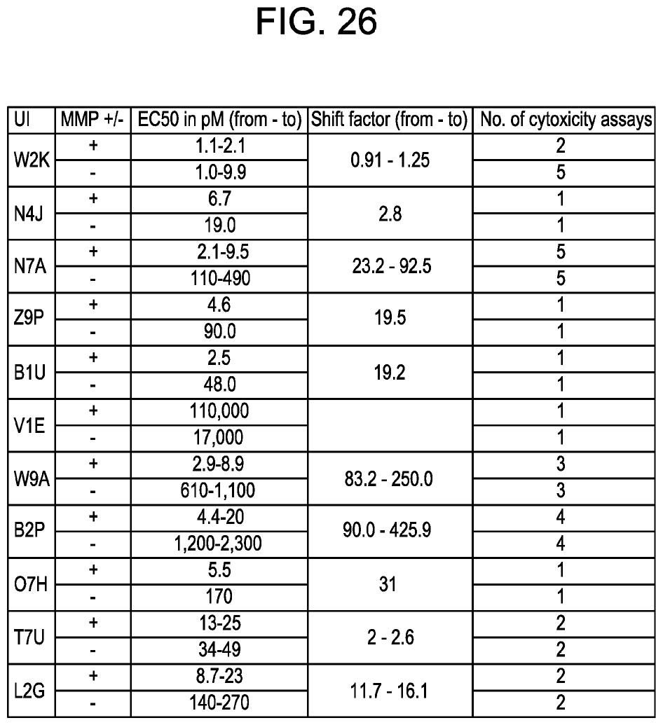

Bispecific binding constructs were applied to in vitro TDCC assays to determine the activity difference between the non-digested constructs versus the MMP-9 digested bispecific binding constructs (Error! Reference source not found. s 20-25). Bispecific binding constructs were incubated with recombinant MMP-9 at a 1:1 molar ratio for 18 h at 37° C. (or PBS as a control). CHO cells transfected with the target antigen (target cells) were labeled using Vybrant DiO prior to assay setup and human pan T cells (effector cells) were isolated using a Pan T-cell isolation kit (Miltenyl) from human PBMCs donated by voluntary, healthy donors. Bispecific binding construct dilution series in combination with target and effector cell populations were incubated at an effector:target ratio of 10:1 and incubated for 48 hours at 37° C., 5% CO 2, 95% humidity. After 48 hours cells were centrifuged, stained with propidium idodide (PI) and applied to flow cytometry. The percentage of cells positive for Vybrant DiO and propidium iodide (PI) were plotted against the corresponding bispecific binding construct concentration to determine the EC₅₀ value of the dose-response curves for activity comparison. EC₅₀ values and the factor (fold potency difference) was calculated by dividing the EC₅₀ of the MMP9-incubated bispecific binding construct by the EC₅₀ of the PBS-incubated bispecific binding construct. The range of EC₅₀ values, number of assays and factors (fold difference between PBS and MMP9 incubated bispecific binding constructs) is shown in FIG. 26. A non-MMP9 cleavable bispecific binding construct (W2K) was used as a reference.

Each and every reference cited herein is incorporated herein by reference in its entirety for all purposes.

The present invention is not to be limited in scope by the specific embodiments described herein, which are intended as single illustrations of individual embodiments of the invention, and functionally equivalent methods and components of the invention. Indeed, various modifications of the invention, in addition to those shown and described herein will become apparent to those skilled in the art from the foregoing description and accompanying drawings. Such modifications are intended to fall within the scope of the claims.

SEQUENCES

```
Exemplary Linker Sequences
GGGGS (SEQ ID NO: 1)
GGGGSGGGGS (SEQ ID NO: 2)
GGGGSGGGGSGGGGS (SEQ ID NO: 3)
GGGGSGGGGSGGGGSGGGGS (SEQ ID NO: 4)
GGGGSGGGGSGGGGSGGGGSGGGGS (SEQ ID NO: 5)
GGGGQ (SEQ ID NO: 6)
GGGGQGGGGQ (SEQ ID NO: 7)
GGGGQGGGGQGGGGQ (SEQ ID NO: 8)
GGGGQGGGGQGGGGQGGGGQ (SEQ ID NO: 9)
GGGGQGGGGQGGGGQGGGGQGGGGQ (SEQ ID NO: 10)
GGGGSAAA (SEQ ID NO: 11)
TVAAP (SEQ ID NO: 12)
ASTKGP (SEQ ID NO: 13)
AAA (SEQ ID NO: 14)
GGNGT (SEQ ID NO: 15)
YGNGT (SEQ ID NO: 16)
Fc Regions (SEQ ID NOs: 56-59)
IgG1 -----------------------------------------------
IgG2 -----------------------------------------------
IgG3 ELKTPLGDTTHTCPRCPEPKSCDTPPPCPRCPEPKSCDTPPPCPRCP
IgG4 -----------------------------------------------
                          225 235 245 255 265 275
                            * * * * * *
IgG1 EPKSCDKTHTCPPCPAPELLGGPSVFLFPPKPKDTLMISRTPEVTCVVVDVSHEDPEVKF
IgG2 ERKCCVE---CPPCPAPPVA-GPSVFLFPPKPKDTLMISRTPEVTCVVVDVSHEDPEVQF
IgG3 EPKSCDTPPPCPRCPAPELLGGPSVFLFPPKPKDTLMISRTPEVTCVVVDVSHEDPEVQF
IgG4 ESKYG---PPCPSCPAPEFLGGPSVFLFPPKPKDTLMISRTPEVTCVVVDVSQEDPEVQF
                          285 295 305 315 325 335
                            * * * * * *
IgG1 NWYVDGVEVHNAKTKPREEQYNSTYRVVSVLTVLHQDWLNGKEYKCKVSNKALPAPIEKT
IgG2 NWYVDGMEVHNAKTKPREEQFNSTFRVVSVLTVVHQDWLNGKEYKCKVSNKGLPAPIEKT
IgG3 KWYVDGVEVHNAKTKPREEQYNSTFRVVSVLTVLHQDWLNGKEYKCKVSNKALPAPIEKT
IgG4 NWYVDGVEVHNAKTKPREEQFNSTYRVVSVLTVLHQDWLNGKEYKCKVSNKGLPSSIEKT
                          345 355 365 375 385 395
                            * * * * * *
IgG1 ISKAKGQPREPQVYTLPPSREEMTKNQVSLTCLVKGFYPSDIAVEWESNGQPENNYKTTP
IgG2 ISKTKGQPREPQVYTLPPSREEMTKNQVSLTCLVKGFYPSDIAVEWESNGQPENNYKTTP
IgG3 ISKTKGQPREPQVYTLPPSREEMTKNQVSLTCLVKGFYPSDIAVEWESSGQPENNYNTTP
IgG4 ISKAKGQPREPQVYTLPPSQEEMTKNQVSLTCLVKGFYPSDIAVEWESNGQPENNYKTTP
                          405 415 425 435 445
                            * * * * *
IgG1 PVLDSDGSFFLYSKLTVDKSRWQQGNVFSCSVMHEALHNHYTQKSLSLSPGK (SEQ ID NO: 56)
IgG2 PMLDSDGSFFLYSKLTVDKSRWQQGNVFSCSVMHEALHNHYTQKSLSLSPGK (SEQ ID NO: 57)
IgG3 PMLDSDGSFFLYSKLTVDKSRWQQGNIFSCSVMHEALHNRFTQKSLSLSPGK (SEQ ID NO: 58)
IgG4 PVLDSDGSFFLYSRLTVDKSRWQEGNVFSCSVMHEALHNHYTQKSLSLSLGK (SEQ ID NO: 59)
SEQ ID NO: 60 Amino acid sequence of the mature human CD3ϵ
QDGNEEMGG ITQTPYKVSI SGTTVILTCP QYPGSEILWQ HNDKNIGGDE DDKNIGSDE DHLSLKEFSEL
EQSGYYVCYP RGSKPEDANF YLYLRARVCE NCMEMDVMSV ATIVIVDIC ITGGLLLLVYY WSKNRKAKAK
PVTRGAGAGG RQRGQNKERP PPVPNPDYEP IRKGQRDLYS GLNQRRI
SEQ ID NO: 61 Amino acid sequence of the mature CD3ϵ of cynomolgus monkey
QDGNEEMGS ITQTPYQVSI SGTTVILTCS QHLGSEAQWQ HNGKNKGDSG DQLFLPEFSE MEQSGYYVCY
PRGSNPEDAS HHLYLKARVC ENCMEMDVMA VATIVIVDIC ITLGLLLLVY YWSKNRKAKA KPVTRGAGAG
GRQRGQNKER PPPVPNPDYE PIRKGQQDLY SGLNQRRI
SEQ ID NO: 62 Amino acid sequence of the extracellular domain of human CD3ϵ
QDGNEEMGG ITQTPYKVSI SGTTVILTCP QYPGSEILWQ HNDKNIGGDE DDKNIGSDED HLSLKEFSEL
EQSGYYVCYP RGSKPEDANF YLYLRARVCE NCMEMDVMS
SEQ ID NO: 63 Amino acids 1-27 of human CD3ϵ
QDGNEEMGGITQTPYKVSISGTTVILT
SEQ ID NO: 64 Amino acids 1-6 of human CD3ϵ
QDGNEE
Anti-CD3 VH (SEQ ID NO: 65)
EVQLVESGGGLVQPGGSLKLSCAASGFTFNKYAMNWVRQAPGKGLEWVARIRSKYNNYATYYADSVKDRFTISRDD
SKNTAYLQMNNLKTEDTAVYYCVRHGNFGNSYISYWAYWGQGTLVIVSS
Anti-CD3 VL (SEQ ID NO: 66)
QTVVTQEPSLTVSPGGTVTLTCGSSTGAVTSGNYPNWVQQKPGQAPRGLIGGTKFLAPGTPARFSGSLLGGKAALT
LSGVQPEDEAEYYCVLWYSNRWVFGGGTKLTVL
Anti-CD3 VH including W103C cysteine clamp (Kabat) (SEQ ID NO: 67)
EVQLVESGGGLVQPGGSLKLSCAASGFTFNKYAMNWVRQAPGKGLEWVARIRSKYNNYATYYADSVKDRFTISRDD
SKNTAYLQMNNLKTEDTAVYYCVRHGNFGNSYISYWAYCGQGTLVTVSS
Anti-CD3 VL including A43C cysteine clamp (Kabat) (SEQ ID NO: 68)
QTVVTQEPSLTVSPGGTVTLTCGSSTGAVTSGNYPNWVQQKPGQCPRGLIGGTKFLAPGTPARFSGSLLGGKAALT
LSGVQPEDEAEYYCVLWYSNRWVFGGGTKLTVL
Anti-CD3 VH CDR1 (SEQ ID NO: 69)
KYAMN
Anti-CD3 VH CDR2 (SEQ ID NO: 70)
RIRSKYNNYATYYADSVKD
Anti-CD3 VH CDR3 (SEQ ID NO: 71)
HGNFGNSYISYWAY
Anti-CD3 VL CDR1 (SEQ ID NO: 72)
GSSTGAVTSGNYPN
Anti-CD3 VL CDR2 (SEQ ID NO: 73)
GTKFLAP
Anti-CD3 VL CDR3 (SEQ ID NO: 74)
VLWYSNRWV
Anti-MSLN VH (SEQ ID NO: 75)
QVQLVESGGGLVKPGGSLRLSCAASGFTFSDYYMTWIRQAPGKGLEWLSYISSSGSTIYYADSVKGRFTISRDNAK
NSLFLQMNSLRAEDTAVYYCARDRNSHFDYWGQGTLVTVSS
Anti-MSLN VL (SEQ ID NO: 76)
DIQMTQSPSSVSASVGDRVTITCRASQGINTWLAWYQQKPGKAPKWYGASGLQSGVPSRFSGSGSGTDFTLTISSL
QPEDFATYYCQQAKSFPRTFGQGTKVEIK
Anti-MSLN VH including G44C cysteine clamp (Kabat) (SEQ ID NO: 77)
QVQLVESGGGLVKPGGSLRLSCAASGFTFSDYYMTWIRQAPGKCLEWLSYISSSGSTIYYADSVKGRFTISRDNAK
NSLFLQMNSLRAEDTAVYYCARDRNSHFDYWGQGTLVTVSS
Anti-MSLN VL including Q100C cysteine clamp (Kabat) (SEQ ID NO: 78)
DIQMTQSPSSVSASVGDRVTITCRASQGINTWLAWYQQKPGKAPKWYGASGLQSGVPSRFSGSGSGTDFTLTISSL
QPEDFATYYCQQAKSFPRTFGCGTKVEIK
Anti-MSLN VH CDR1 (SEQ ID NO: 79)
DYYMT
Anti-MSLN VH CDR2 (SEQ ID NO: 80)
YISSSGSTIYYADSVKG
Anti-MSLN VH CDR3 (SEQ ID NO: 81)
DRNSHFDY
Anti-MSLN VL CDR1 (SEQ ID NO: 82)
RASQGINTWLA
Anti-MSLN VL CDR2 (SEQ ID NO: 83)
GASGLQS
Anti-MSLN VL CDR3 (SEQ ID NO: 84)
QQAKSFPRT
scFc (SEQ ID NO: 85)
DKTHTCPPCPAPELLGGPSVFLFPPKPKDTLMISRTPEVTCVVVDVSHEDPEVKFNWYVDGVEVHNAKTKPCEEQY
GSTYRCVSVLTVLHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNHYTQKSLSLSPGKG
GGGSGGGGSGGGGSGGGGSGGGGSGGGGSDKTHTCPPCPAPELLGGPSVFLFPPKPKDTLMISRTPEVTCVVVDVS
HEDPEVKFNWYVDGVEVHNAKTKPCEEQYGSTYRCVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLIVDKSRWQQ
GNVFSCSVMHEALHNHYTQKSLSLSPGK
scFc subdomain1 (SEQ ID NO: 86)
DKTHTCPPCPAPELLGGPSVFLFPPKPKDTLMISRTPEVTCVVVDVSHEDPEVKFNWYVDGVEVHNAKTKPCEEQY
GSTYRCVSVLTVLHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNHYTQKSLSLSPGK
scFc subdomain2 (SEQ ID NO: 87)
DKTHTCPPCPAPELLGGPSVFLFPPKPKDTLMISRTPEVTCVVVDVSHEDPEVKFNWYVDGVEVHNAKTKPCEEQY
GSTYRCVSVLTVLHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNHYTQKSLSLSPGK
W2K (SEQ ID NO: 88)
QVQLVESGGGLVKPGGSLRLSCAASGFTFSDYYMTWIRQAPGKGLEWLSYISSSGSTIYYADSVKGRFTISRDNAK
NSLFLQMNSLRAEDTAVYYCARDRNSHFDYWGQGTLVTVSSGGGGSGGGGSGGGGSDIQMTQSPSSVSASVGDRVT
ITCRASQGINTWLAWYQQKPGKAPKLLIYGASGLQSGVPSRFSGSGSGTDFTLTISSLQPEDFATYYCQQAKSFPR
TFGQGTKVEIKSGGGGSEVQLVESGGGLVQPGGSLKLSCAASGFTFNKYAMNWVRQAPGKGLEWVARIRSKYNNYA
TYYADSVKDRFTISRDDSKNTAYLQMNNLKTEDTAVYYCVRHGNFGNSYISYWAYWGQGTLVTVSSGGGGSGGGGS
GGGGSQTVVTQEPSLTVSPGGIVTLTCGSSTGAVTSGNYPNWVQQKPGQAPRGLIGGTKFLAPGTPARFSGSLLGG
KAALTLSGVQPEDEAEYYCVLWYSNRWVFGGGTKLTVLGGGGDKTHTCPPCPAPELLGGPSVFLFPPKPKDTLMIS
RTPEVTCVVVDVSHEDPEVKFNWYVDGVEVHNAKTKPCEEQYGSTYRCVSVLIVLHQDWLNGKEYKCKVSNKALPA
PIEKTISKAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSGGGGSGGGGSDKTHT
CPPCPAPELLGGPSVFLFPPKPKDTLMISRTPEVTCVVVDVSHEDPEVKFNWYVDGVEVHNAKTKPCEEQYGSTYR
CVSVLIVLHQDWLNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSLSLSPGK
N4J (SEQ ID NO: 89)
EVQLVESGGGLVQPGGSLKLSCAASGFTFNKYAMNWVRQAPGKGLEWVARIRSKYNNYATYYADSVKDRFTISRDD
SKNTAYLQMNNLKTEDTAVYYCVRHGNFGNSYISYWAYWGQGTLVTVSSGGGGSGGGGSGGGGSGGGGSQVQLVES
GGGLVKPGGSLRLSCAASGFTFSDYYMTWIRQAPGKCLEWLSYISSSGSTIYYADSVKGRFTISRDNAKNSLFLQM
NSLRAEDTAVYYCARDRNSHFDYWGQGTLVTVSSGGGGSGGGGSGGGGSGGGGSQTVVTQEPSLTVSPGGTVTLTC
GSSTGAVTSGNYPNWVQQKPGQAPRGLIGGTKFLAPGTPARFSGSLLGGKAALTLSGVQPEDEAEYYCVLWYSNRW
VFGGGTKLTVLGGGGSGGPLGMLSQSGGGGSDIQMTQSPSSVSASVGDRVTITCRASQGINTWLAWYQQKPGKAPK
LLIYGASGLQSGVPSRFSGSGSGTDFTLTISSLQPEDFATYYCQQAKSFPRTFGCGTKVEIKLTVLGGGGDKTHTC
PPCPAPELLGGPSVFLFPPKPKDTLMISRTPEVTCVVVDVSHEDPEVKFNWYVDGVEVHNAKTKPCEEQYGSTYRC
VSVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPGKGGGGSGG
GGSGGGGSGGGGSGGGGSGGGGSDKTHTCPPCPAPELLGGPSVFLFPPKPKDTLMISRTPEVTCVVVDVSHEDPEV
KFNWYVDGVEVHNAKTKPCEEQYGSTYRCVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGK
N7A (SEQ ID NO: 90)
QDGNEEGGPLGMLSQSGEVQLVESGGGLVQPGGSLKLSCAASGFTFNKYAMNWVRQAPGKGLEWVARIRSKYNNYA
TYYADSVKDRFTISRDDSKNTAYLQMNNLKTEDTAVYYCVRHGNFGNSYISYWAYCGQGTLVTVSSGGPLGMLSQS
GQVQLVESGGGLVKPGGSLRLSCAASGFTFSDYYMTWIRQAPGKCLEWLSYISSSGSTIYYADSVKGRFTISRDNA
KNSLFLQMNSLRAEDTAVYYCARDRNSHFDYWGQGTLVTVSSGGGGSGGGGSGGGGSQTVVTQEPSLTVSPGGTVT
LTCGSSTGAVTSGNYPNWVQQKPGQCPRGLIGGTKFLAPGTPARFSGSLLGGKAALTLSGVQPEDEAEYYCVLWYS
NRWVFGGGTKLTVLSGGGPLGMLSQSGGGDIQMTQSPSSVSASVGDRVTITCRASQGINTWLAWYQQKPGKAPKLL
IYGASGLQSGVPSRFSGSGSGTDFTLTISSLQPEDFATYYCQQAKSFPRTFGCGTKVEIKSGPLGMLSQSGDKTHT
CPPCPAPELLGGPSVFLFPPKPKDTLMISRTPEVTCVVVDVSHEDPEVKFNWYVDGVEVHNAKTKPCEEQYGSTYR
CVSVLTVLHQDWLNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSLSLSPGKGGGGSG
GGGSGGGGSGGGGSGGGGSGGGGSDKTHTCPPCPAPELLGGPSVFLFPPKPKDTLMISRTPEVTCVVVDVSHEDPE
VKFNWYVDGVEVHNAKTKPCEEQYGSTYRCVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
B1U (SEQ ID NO: 91)
QDGNEESGGPLGMLSQSGEVQLVESGGGLVQPGGSLKLSCAASGFTFNKYAMNWVRQAPGKGLEWVARIRSKYNNY
ATYYADSVKDRFTISRDDSKNTAYLQMNNLKTEDTAVYYCVRHGNFGNSYISYWAYCGQGTLVTVSSSGGPLGMLS
QSGDKTHTCPPCPAPELLGGPSVFLFPPKPKDTLMISRTPEVTCVVVDVSHEDPEVKFNWYVDGVEVHNAKTKPCE
EQYGSTYRCVSVLIVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEALHNHYTQKSLSLSP
GKSGGPLGMLSQSGQVQLVESGGGLVKPGGSLRLSCAASGFTFSDYYMTWIRQAPGKCLEWLSYISSSGSTIYYAD
SVKGRFTISRDNAKNSLFLQMNSLRAEDTAVYYCARDRNSHFDYWGQGTLVTVSSGGGGSGGGGSGGGGSGGGGSG
GGGSGGGGSQTVVTQEPSLTVSPGGTVTLTCGSSTGAVTSGNYPNWVQQKPGQCPRGLIGGTKFLAPGTPARFSGS
LLGGKAALTLSGVQPEDEAEYYCVLWYSNRWVFGGGTKLTVLSGGPLGMLSQSGDKTHTCPPCPAPELLGGPSVFL
FPPKPKDTLMISRTPEVTCVVVDVSHEDPEVKFNWYVDGVEVHNAKTKPCEEQYGSTYRCVSVLTVLHQDWLNGKE
YKCKVSNKALPAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGKSGGPLGMLSQSGDIQMTQSPSSV
SASVGDRVTITCRASQGINTWLAWYQQKPGKAPKLLIYGASGLQSGVPSRFSGSGSGTDFTLTISSLQPEDFATYY
CQQAKSFPRTFGCGTKVEIK
Z9P (SEQ ID NO: 92)
QDGNEESGGPLGMLSQSGEVQLVESGGGLVQPGGSLKLSCAASGFTFNKYAMNWVRQAPGKGLEWVARIRSKYNNY
ATYYADSVKDRFTISRDDSKNTAYLQMNNLKTEDTAVYYCVRHGNFGNSYISYWAYCGQGTLVTVSSSGGPLGMLS
QSGDKTHTCPPCPAPELLGGPSVFLFPPKPKDTLMISRTPEVTCVVVDVSHEDPEVKFNWYVDGVEVHNAKTKPCE
EQYGSTYRCVSVLIVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEALHNHYTQKSLSLSP
GKSGGPLGMLSQSGQVQLVESGGGLVKPGGSLRLSCAASGFTFSDYYMTWIRQAPGKCLEWLSYISSSGSTIYYAD
SVKGRFTISRDNAKNSLFLQMNSLRAEDTAVYYCARDRNSHFDYWGQGTLVTVSSGGGGSGGGGSGGGGSGGGGSG
GGGSGGGGSGGGGSGGGGSGGGGSGGGGSGGGGSGGGGSQTVVTQEPSLTVSPGGTVTLTCGSSTGAVTSGNYPNW
VQQKPGQCPRGLIGGTKFLAPGTPARFSGSLLGGKAALTLSGVQPEDEAEYYCVLWYSNRWVFGGGTKLTVLSGGP
LGMLSQSGDKTHTCPPCPAPELLGGPSVFLFPPKPKDTLMISRTPEVTCVVVDVSHEDPEVKFNWYVDGVEVHNAK
TKPCEEQYGSTYRCVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYTQKS
LSLSPGKSGGPLGMLSQSGDIQMTQSPSSVSASVGDRVTITCRASQGINTWLAWYQQKPGKAPKLLIYGASGLQSG
VPSRFSGSGSGTDFTLTISSLQPEDFATYYCQQAKSFPRTFGCGTKVEIK
V1E (SEQ ID NO: 93)
QDGNEESGGPLGMLSQSGEVQLVESGGGLVQPGGSLKLSCAASGFTFNKYAMNWVRQAPGKGLEWVARIRSKYNNY
ATYYADSVKDRFTISRDDSKNTAYLQMNNLKTEDTAVYYCVRHGNFGNSYISYWAYCGQGTLVTVSSSGGPLGMLS
QSGDKTHTCPPCPAPELLGGPSVFLFPPKPKDTLMISRTPEVTCVVVDVSHEDPEVKFNWYVDGVEVHNAKTKPCE
EQYGSTYRCVSVLIVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEALHNHYTQKSLSLSP
GKSGGPLGMLSQSGQVQLVESGGGLVKPGGSLRLSCAASGFTFSDYYMTWIRQAPGKCLEWLSYISSSGSTIYYAD
SVKGRFTISRDNAKNSLFLQMNSLRAEDTAVYYCARDRNSHFDYWGQGTLVTVSSGGGGSGGGGSGGGGSQTVVTQ
EPSLTVSPGGTVTLTCGSSTGAVTSGNYPNWVQQKPGQCPRGLIGGTKFLAPGTPARFSGSLLGGKAALTLSGVQP
EDEAEYYCVLWYSNRWVFGGGTKLTVLSGGPLGMLSQSGDKTHTCPPCPAPELLGGPSVFLFPPKPKDTLMISRTP
EVTCVVVDVSHEDPEVKFNWYVDGVEVHNAKTKPCEEQYGSTYRCVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKSGGPLGMLSQSGDIQMTQSPSSVSASVGDRVTITCRAS
QGINTWLAWYQQKPGKAPKLLIYGASGLQSGVPSRFSGSGSGTDFTLTISSLQPEDFATYYCQQAKSFPRTFGCGT
KVEIK
B2P (SEQ ID NO: 94)
QDGNEESGDAHKSEVAHRFKDLGEENFKALVLIAFAQYLQQCPFEDHVKLVNEVTEFAKTCVADESAENCDKSLHT
LFGDKLCTVATLRETYGEMADCCAKQEPERNECFLQHKDDNPNLPRLVRPEVDVMCTAFHDNEETFLKKYLYEIAR
RHPYFYAPELLFFAKRYKAAFTECCQAADKAACLLPKLDELRDEGKASSAKQRLKCASLQKFGERAFKAWAVARLS
QRFPKAEFAEVSKLVTDLTKVHTECCHGDLLECADDRADLAKYICENQDSISSKLKECCEKPLLEKSHCIAEVEND
EMPADLPSLAADFVESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVGSKCCKHPEAKRMPC
AEDYLSVVLNQLCVLHEKTPVSDRVTKCCTESLVNRRPCFSALEVDETYVPKEFNAETFTFHADICTLSEKERQIK
KQTALVELVKHKPKATKEQLKAVMDDFAAFVEKCCKADDKETCFAEEGKKLVAASQAALGLGGGGSGGGGSGGGGS
GGGGSGGGGSGGGGSGGPLGMLSQSGEVQLVESGGGLVQPGGSLKLSCAASGFTFNKYAMNWVRQAPGKGLEWVAR
IRSKYNNYATYYADSVKDRFTISRDDSKNTAYLQMNNLKTEDTAVYYCVRHGNFGNSYISYWAYCGQGTLVTVSSG
GPLGMLSQSGQVQLVESGGGLVKPGGSLRLSCAASGFTFSDYYMTWIRQAPGKCLEWLSYISSSGSTIYYADSVKG
RFTISRDNAKNSLFLQMNSLRAEDTAVYYCARDRNSHFDYWGQGTLVTVSSGGGGSGGGGSGGGGSQTVVTQEPSL
TVSPGGTVTLTCGSSTGAVTSGNYPNWVQQKPGQCPRGLIGGTKFLAPGTPARFSGSLLGGKAALTLSGVQPEDEA
EYYCVLWYSNRWVFGGGTKLTVLSGGGPLGMLSQSGGGDIQMTQSPSSVSASVGDRVTITCRASQGINTWLAWYQQ
KPGKAPKLLIYGASGLQSGVPSRFSGSGSGTDFTLTISSLQPEDFATYYCQQAKSFPRTFGCGTKVEIKSGPLGML
SQSGDKTHTCPPCPAPELLGGPSVFLFPPKPKDTLMISRTPEVTCVVVDVSHEDPEVKFNWYVDGVEVHNAKTKPC
EEQYGSTYRCVSVLIVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEALHNHYTQKSLSLS
PGKGGGGSGGGGSGGGGSGGGGSGGGGSGGGGSDKTHTCPPCPAPELLGGPSVFLFPPKPKDTLMISRTPEVTCVV
VDVSHEDPEVKFNWYVDGVEVHNAKTKPCEEQYGSTYRCVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
W9A (SEQ ID NO: 95)
QDGNEEMGGITQTPYKVSISGTTVILTSGDAHKSEVAHRFKDLGEENFKALVLIAFAQYLQQCPFEDHVKLVNEVT
EFAKTCVADESAENCDKSLHTLFGDKLCTVATLRETYGEMADCCAKQEPERNECFLQHKDDNPNLPRLVRPEVDVM
CTAFHDNEETFLKKYLYEIARRHPYFYAPELLFFAKRYKAAFTECCQAADKAACLLPKLDELRDEGKASSAKQRLK
CASLQKFGERAFKAWAVARLSQRFPKAEFAEVSKLVTDLTKVHTECCHGDLLECADDRADLAKYICENQDSISSKL
KECCEKPLLEKSHCIAEVENDEMPADLPSLAADFVESKDVCKNYAEAKDVFLGMFLYEYARRHPDYSVVLLLRLAK
TYETTLEKCCAAADPHECYAKVFDEFKPLVEEPQNLIKQNCELFEQLGEYKFQNALLVRYTKKVPQVSTPTLVEVS
RNLGKVGSKCCKHPEAKRMPCAEDYLSVVLNQLCVLHEKTPVSDRVTKCCTESLVNRRPCFSALEVDETYVPKEFN
AETFTFHADICTLSEKERQIKKQTALVELVKHKPKATKEQLKAVMDDFAAFVEKCCKADDKETCFAEEGKKLVAAS
QAALGLGGGGSGGGGSGGGGSGGGGSGGGGSGGGGSGGPLGMLSQSGEVQLVESGGGLVQPGGSLKLSCAASGFTF
NKYAMNWVRQAPGKGLEWVARIRSKYNNYATYYADSVKDRFTISRDDSKNTAYLQMNNLKTEDTAVYYCVRHGNFG
NSYISYWAYCGQGTLVTVSSGGPLGMLSQSGQVQLVESGGGLVKPGGSLRLSCAASGFTFSDYYMTWIRQAPGKCL
EWLSYISSSGSTIYYADSVKGRFTISRDNAKNSLFLQMNSLRAEDTAVYYCARDRNSHFDYWGQGTLVTVSSGGGG
SGGGGSGGGGSQTVVTQEPSLTVSPGGIVTLTCGSSTGAVTSGNYPNWVQQKPGQCPRGLIGGTKFLAPGTPARFS
GSLLGGKAALTLSGVQPEDEAEYYCVLWYSNRWVFGGGTKLTVLSGGGPLGMLSQSGGGDIQMTQSPSSVSASVGD
RVTITCRASQGINTWLAWYQQKPGKAPKLLIYGASGLQSGVPSRFSGSGSGTDFTLTISSLQPEDFATYYCQQAKS
FPRTFGCGTKVEIKSGPLGMLSQSGDKTHTCPPCPAPELLGGPSVFLFPPKPKDTLMISRTPEVTCVVVDVSHEDP
EVKFNWYVDGVEVHNAKTKPCEEQYGSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVF
SCSVMHEALHNHYTQKSLSLSPGKGGGGSGGGGSGGGGSGGGGSGGGGSGGGGSDKTHTCPPCPAPELLGGPSVFL
FPPKPKDTLMISRTPEVTCVVVDVSHEDPEVKFNWYVDGVEVHNAKTKPCEEQYGSTYRCVSVLTVLHQDWLNGKE
YKCKVSNKALPAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
L2G (SEQ ID NO: 96)
QDGNEESGDKTHTCPPCPAPELLGGPSVFLFPPKPKDTLMISRTPEVTCVVVDVSHEDPEVKFNWYVDGVEVHNAK
TKPCEEQYGSTYRCVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYTQKS
LSLSPGKGGGGSGGGGSGGGGSGGGGSGGGGSGGGGSDKTHTCPPCPAPELLGGPSVFLFPPKPKDTLMISRTPEV
TCVVVDVSHEDPEVKFNWYVDGVEVHNAKTKPCEEQYGSTYRCVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGKGGGGSGGGGSGGGGSGGGGSGGGGSGGGGSGGPLGMLSQS
GEVQLVESGGGLVQPGGSLKLSCAASGFTFNKYAMNWVRQAPGKGLEWVARIRSKYNNYATYYADSVKDRFTISRD
DSKNTAYLQMNNLKTEDTAVYYCVRHGNFGNSYISYWAYCGQGTLVTVSSGGPLGMLSQSGQVQLVESGGGLVKPG
GSLRLSCAASGFTFSDYYMTWIRQAPGKCLEWLSYISSSGSTIYYADSVKGRFTISRDNAKNSLFLQMNSLRAEDT
AVYYCARDRNSHFDYWGQGTLVTVSSGGGGSGGGGSGGGGSQTVVTQEPSLTVSPGGTVTLTCGSSTGAVTSGNYP
NWVQQKPGQCPRGLIGGTKFLAPGTPARFSGSLLGGKAALTLSGVQPEDEAEYYCVLWYSNRWVFGGGTKLTVLSG
GGPLGMLSQSGGGDIQMTQSPSSVSASVGDRVTITCRASQGINTWLAWYQQKPGKAPKLLIYGASGLQSGVPSRFS
GSGSGTDFTLTISSLQPEDFATYYCQQAKSFPRTFGCGTKVEIK
T7U (SEQ ID NO: 97)
DKTHTCPPCPAPELLGGPSVFLFPPKPKDTLMISRTPEVTCVVVDVSHEDPEVKFNWYVDGVEVHNAKTKPCEEQY
GSTYRCVSVLTVLHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNHYTQKSLSLSPGKG
GGGSGGGGSGGGGSGGGGSGGGGSGGGGSDKTHTCPPCPAPELLGGPSVFLFPPKPKDTLMISRTPEVTCVVVDVS
HEDPEVKFNWYVDGVEVHNAKTKPCEEQYGSTYRCVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDGSFFLYSKLIVDKSRWQQ
GNVFSCSVMHEALHNHYTQKSLSLSPGKGGGGSGGGGSGGGGSGGGGSGGGGSGGGGSGGPLGMLSQSGEVQLVES
GGGLVQPGGSLKLSCAASGFTFNKYAMNWVRQAPGKGLEWVARIRSKYNNYATYYADSVKDRFTISRDDSKNTAYL
QMNNLKTEDTAVYYCVRHGNFGNSYISYWAYCGQGTLVTVSSGGPLGMLSQSGQVQLVESGGGLVKPGGSLRLSCA
ASGFTFSDYYMTWIRQAPGKCLEWLSYISSSGSTIYYADSVKGRFTISRDNAKNSLFLQMNSLRAEDTAVYYCARD
RNSHFDYWGQGTLVTVSSGGGGSGGGGSGGGGSQTVVTQEPSLTVSPGGTVTLTCGSSTGAVTSGNYPNWVQQKPG
QCPRGLIGGTKFLAPGTPARFSGSLLGGKAALTLSGVQPEDEAEYYCVLWYSNRWVFGGGTKLTVLSGGGPLGMLS
QSGGGDIQMTQSPSSVSASVGDRVTITCRASQGINTWLAWYQQKPGKAPKLLIYGASGLQSGVPSRFSGSGSGTDF
TLTISSLQPEDFATYYCQQAKSFPRTFGCGTKVEIK
O7H (SEQ ID NO: 98)
DAHKSEVAHRFKDLGEENFKALVLIAFAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTAFHDNEETFLKKYLYEIARRHPYFYAP
ELLFFAKRYKAAFTECCQAADKAACLLPKLDELRDEGKASSAKQRLKCASLQKFGERAFKAWAVARLSQRFPKAEF
AEVSKLVTDLTKVHTECCHGDLLECADDRADLAKYICENQDSISSKLKECCEKPLLEKSHCIAEVENDEMPADLPS
LAADFVESKDVCKNYAEAKDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCKHPEAKRMPCAEDYLSVV
LNQLCVLHEKTPVSDRVTKCCTESLVNRRPCFSALEVDETYVPKEFNAETFTFHADICTLSEKERQIKKQTALVEL
VKHKPKATKEQLKAVMDDFAAFVEKCCKADDKETCFAEEGKKLVAASQAALGLGGGGSGGGGSGGGGSGGGGSGGG
GSGGGGSGGPLGMLSQSGEVQLVESGGGLVQPGGSLKLSCAASGFTFNKYAMNWVRQAPGKGLEWVARIRSKYNNY
ATYYADSVKDRFTISRDDSKNTAYLQMNNLKTEDTAVYYCVRHGNFGNSYISYWAYCGQGTLVTVSSGGPLGMLSQ
SGQVQLVESGGGLVKPGGSLRLSCAASGFTFSDYYMTWIRQAPGKCLEWLSYISSSGSTIYYADSVKGRFTISRDN
AKNSLFLQMNSLRAEDTAVYYCARDRNSHFDYWGQGTLVTVSSGGGGSGGGGSGGGGSQTVVTQEPSLTVSPGGTV
TLTCGSSTGAVTSGNYPNWVQQKPGQCPRGLIGGTKFLAPGTPARFSGSLLGGKAALTLSGVQPEDEAEYYCVLWY
SNRWVFGGGTKLTVLSGGGPLGMLSQSGGGDIQMTQSPSSVSASVGDRVTITCRASQGINTWLAWYQQKPGKAPKL
LIYGASGLQSGVPSRFSGSGSGTDFTLTISSLQPEDFATYYCQQAKSFPRTFGCGTKVEIKSGPLGMLSQSGDKTH
TCPPCPAPELLGGPSVFLFPPKPKDTLMISRTPEVTCVVVDVSHEDPEVKFNWYVDGVEVHNAKTKPCEEQYGSTY
RCVSVLTVLHQDWLNGKEYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALHNHYTQKSLSLSPGKGGGGS
GGGGSGGGGSGGGGSGGGGSGGGGSDKTHTCPPCPAPELLGGPSVFLFPPKPKDTLMISRTPEVTCVVVDVSHEDP
EVKFNWYVDGVEVHNAKTKPCEEQYGSTYRCVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVF
SCSVMHEALHNHYTQKSLSLSPGK
```

What is claimed is:

1. A bispecific binding construct comprising a polypeptide chain comprising an amino acid sequence having the formula VH1-L1-VH2-L2-VL1-L3-VL2, wherein VH1 and VH2 comprise immunoglobulin heavy chain variable regions, VL1 and VL2 comprise immunoglobulin light chain variable regions, and L1, L2 and L3 are linkers, wherein L1 is at least 10 amino acids, L2 is at least 15 amino acids and L3 is at least 10 amino acids, wherein L1 or L3 comprises a protease cleavage site, and wherein the bispecific binding construct can bind to an immune effector cell and a target cell.

2. A bispecific binding construct comprising a polypeptide chain comprising an amino acid sequence having the formula VH1-L1-scFc_{Subdomain1}-L2-VH2-L3-VL1-L4-scFc_{Subdomain2}-L5-VL2, wherein VH1 and VH2 comprise immunoglobulin heavy chain variable regions, VL1 and VL2 comprise immunoglobulin light chain variable regions, scFc comprises subdomain 1 or subdomain 2 of an immunoglobulin heavy chain constant domain-2 and an immunoglobulin heavy chain constant domain-3, and L1, L2, L3, L4, and L5 are linkers, wherein L1 is at least 10 amino acids, L2 is at least 10 amino acids, L3 is at least 15 amino acids, L4 is at least 10 amino acids, and L5 is at least 10 amino acids, and wherein L1, L2, L4 and L5 further comprise a protease cleavage site of at least 5 amino acids, and wherein the bispecific binding construct can bind to an immune effector cell and a target cell.

3. The bispecific binding construct of claim 1, wherein the protease cleavage site is present in both L1 and L3.

4. The bispecific binding construct of claim 1, further comprising at least one cysteine clamp.

5. The bispecific binding construct of claim 4, wherein the cysteine clamp is located in a position to facilitate linkage between the VH1 and VL1 subunits, the VH2 and VL2 subunits, or the scFc subunits.

6. The bispecific binding construct of claim 2, further comprising at least one cysteine clamp.

7. The bispecific binding construct of claim 6, wherein the cysteine clamp is located in a position to facilitate linkage between the VH1 and VL1 subunits, the VH2 and VL2 subunits, or the scFc subunits.

8. The bispecific binding construct of claim 1, further comprising a half-life extending moiety.

9. The bispecific binding construct of claim 8, wherein the half-life extending moiety comprises an additional linker and a single chain immunoglobulin Fc region (scFc) encoding a human IgG1, IgG2, or IgG4 antibody.

10. The bispecific binding construct of claim 9, wherein the additional linker comprises a protease cleavage site.

11. The bispecific binding construct of claim 10, wherein the scFc polypeptide chain comprises one or more alterations that inhibit Fc gamma receptor (FcγR) binding and/or one or more alterations that extends half-life.

12. The bispecific binding construct of claim 1 or 2, wherein the VH1, VH2, VL1, and VL2 all have different sequences.

13. The bispecific binding construct of claim 1 or 2, wherein
a. the VH1 sequence comprises SEQ ID NO: 65 or 67, and the VL1 sequence comprises SEQ ID NO: 66 or 68, and the VH2 sequence comprises SEQ ID NO: 75 or 77, and the VL2 sequence comprises SEQ ID NO: 76 or 78, or
b. the VH1 sequence comprises SEQ ID NO: 75 or 77, and the VL1 sequence comprises SEQ ID NO: 76 or 78, and the VH2 sequence comprises SEQ ID NO: 65 or 67, and the VL2 sequence comprises SEQ ID NO: 66 or 68.

14. The bispecific binding construct of claim 1 or 2, further comprising an additional moiety linked to the VH1 with an additional linker (L0), wherein L0 is at least 5 amino acids in length.

15. The bispecific binding construct of claim 14, wherein the additional moiety is a CDR, or a human serum albumin-linker-CD3_{(a.a. 1-6)}, or a human serum albumin-linker-CD3_{(a.a. 1-27)}, or an scFc-linker-CD3ϵ.

16. The bispecific binding construct of claim 14 or 15, wherein L0 further comprises a protease site.

17. The bispecific binding construct of claim 1 or 2, wherein the linkers are different lengths.

18. The bispecific binding construct of claim 1 or 2, wherein the linkers are the same length.

19. The bispecific binding construct of claim 1, wherein L1 and L2 are the same length.

20. The bispecific binding construct of claim 1, wherein L1 and L3 are the same length.

21. The bispecific binding construct of claim 1, wherein L2 and L3 are the same length.

22. The bispecific binding construct of claim 1, wherein the amino acid sequence of L1 is at least 10 amino acids long, the amino acid sequence of L2 is at least 15 amino acids long, and the amino acid sequence of L3 is at least 15 amino acids long.

23. The bispecific binding construct of claim 1 or 2, wherein the effector cell expresses an effector cell protein that is part of a human T cell receptor (TCR)-CD3 complex.

24. The bispecific binding construct of claim 1 or 2, wherein the effector cell protein is the CD3ϵ chain

25. A nucleic acid encoding the bispecific binding construct of claim 1 or 2.

26. A vector comprising the nucleic acid of claim 25.

27. A host cell comprising the vector of claim 26.

28. A method of manufacturing the bispecific binding construct of claim 1 or 2 comprising (1) culturing a host cell under conditions so as to express the bispecific binding construct and (2) recovering the bispecific binding construct from the cell mass or cell culture supernatant, wherein the host cell comprises one or more nucleic acid(s) encoding bispecific binding construct of claim 1 or 2.

29. A method of treating a cancer patient comprising administering to the patient a therapeutically effective amount of the bispecific binding construct of claim 1 or 2.

30. The method of claim 29, wherein a chemotherapeutic agent, a non-chemotherapeutic anti-neoplastic agent, and/or radiation is administered to the patient concurrently with, before, or after administration of the bispecific binding construct.

31. A pharmaceutical composition comprising the bispecific binding construct of claim 1 or 2.

32. The use of the bispecific binding construct of claim 1 or 2 in the manufacture of a medicament for the prevention, treatment or amelioration of a disease.


SEQUENCE LISTING (110 sequences)

SEQ ID NO: 1 - protein, 5 aa, Artificial Sequence
  Description of Artificial Sequence Synthetic peptide
GGGGS

SEQ ID NO: 2 - protein, 10 aa, Artificial Sequence
  Description of Artificial Sequence Synthetic peptide
GGGGSGGGGS

SEQ ID NO: 3 - protein, 15 aa, Artificial Sequence
  Description of Artificial Sequence Synthetic peptide
GGGGSGGGGSGGGGS

SEQ ID NO: 4 - protein, 20 aa, Artificial Sequence
  Description of Artificial Sequence Synthetic peptide
GGGGSGGGGSGGGGSGGGGS

SEQ ID NO: 5 - protein, 25 aa, Artificial Sequence
  Description of Artificial Sequence Synthetic peptide
GGGGSGGGGSGGGGSGGGGSGGGGS

SEQ ID NO: 6 - protein, 5 aa, Artificial Sequence
  Description of Artificial Sequence Synthetic peptide
GGGGQ

SEQ ID NO: 7 - protein, 10 aa, Artificial Sequence
  Description of Artificial Sequence Synthetic peptide
GGGGQGGGGQ

SEQ ID NO: 8 - protein, 15 aa, Artificial Sequence
  Description of Artificial Sequence Synthetic peptide
GGGGQGGGGQGGGGQ

SEQ ID NO: 9 - protein, 20 aa, Artificial Sequence
  Description of Artificial Sequence Synthetic peptide
GGGGQGGGGQGGGGQGGGGQ

SEQ ID NO: 10 - protein, 25 aa, Artificial Sequence
  Description of Artificial Sequence Synthetic peptide
GGGGQGGGGQGGGGQGGGGQGGGGQ

SEQ ID NO: 11 - protein, 8 aa, Artificial Sequence
  Description of Artificial Sequence Synthetic peptide
GGGGSAAA

SEQ ID NO: 12 - protein, 5 aa, Artificial Sequence
  Description of Artificial Sequence Synthetic peptide
TVAAP

SEQ ID NO: 13 - protein, 6 aa, Artificial Sequence
  Description of Artificial Sequence Synthetic peptide
ASTKGP

SEQ ID NO: 14 - protein, 3 aa, Artificial Sequence
  Description of Artificial Sequence Synthetic peptide
AAA

SEQ ID NO: 15 - protein, 5 aa, Artificial Sequence
  Description of Artificial Sequence Synthetic peptide
GGNGT

SEQ ID NO: 16 - protein, 5 aa, Artificial Sequence
  Description of Artificial Sequence Synthetic peptide
YGNGT

SEQ ID NO: 17 - protein, 8 aa, Artificial Sequence
  Description of Artificial Sequence Synthetic peptide
APMAEGGG

SEQ ID NO: 18 - protein, 8 aa, Artificial Sequence
  Description of Artificial Sequence Synthetic peptide
EAQGDKII

SEQ ID NO: 19 - protein, 8 aa, Artificial Sequence
  Description of Artificial Sequence Synthetic peptide
LAFSDAGP

SEQ ID NO: 20 - protein, 7 aa, Artificial Sequence
  Description of Artificial Sequence Synthetic peptide
YVADAPK

SEQ ID NO: 21 - protein, 5 aa, Artificial Sequence
  Description of Artificial Sequence Synthetic peptide
SGRSA

SEQ ID NO: 22 - protein, 6 aa, Artificial Sequence
  Description of Artificial Sequence Synthetic peptide
GSGRSA

SEQ ID NO: 23 - protein, 5 aa, Artificial Sequence
  Description of Artificial Sequence Synthetic peptide
SGKSA

SEQ ID NO: 24 - protein, 5 aa, Artificial Sequence
  Description of Artificial Sequence Synthetic peptide
SGRSS

SEQ ID NO: 25 - protein, 5 aa, Artificial Sequence
  Description of Artificial Sequence Synthetic peptide
SGRRA

SEQ ID NO: 26 - protein, 5 aa, Artificial Sequence
  Description of Artificial Sequence Synthetic peptide
SGRNA

SEQ ID NO: 27 - protein, 5 aa, Artificial Sequence
  Description of Artificial Sequence Synthetic peptide
SGRKA

SEQ ID NO: 28 - protein, 6 aa, Artificial Sequence
  Description of Artificial Sequence Synthetic peptide
QRGRSA

SEQ ID NO: 29 - protein, 6 aa, Artificial Sequence
  Description of Artificial Sequence Synthetic peptide
TQGAAA

SEQ ID NO: 30 - protein, 6 aa, Artificial Sequence
  Description of Artificial Sequence Synthetic peptide
GAAAAA

SEQ ID NO: 31 - protein, 6 aa, Artificial Sequence
  Description of Artificial Sequence Synthetic peptide
GAGAAG

SEQ ID NO: 32 - protein, 6 aa, Artificial Sequence
  Description of Artificial Sequence Synthetic peptide
AAAAAG

SEQ ID NO: 33 - protein, 6 aa, Artificial Sequence
  Description of Artificial Sequence Synthetic peptide
LCGAAI

SEQ ID NO: 34 - protein, 6 aa, Artificial Sequence
  Description of Artificial Sequence Synthetic peptide
FAQALG

SEQ ID NO: 35 - protein, 6 aa, Artificial Sequence
  Description of Artificial Sequence Synthetic peptide
LAAANP

SEQ ID NO: 36 - protein, 6 aa, Artificial Sequence
  Description of Artificial Sequence Synthetic peptide
LLQANP

SEQ ID NO: 37 - protein, 6 aa, Artificial Sequence
  Description of Artificial Sequence Synthetic peptide
LAAANP

SEQ ID NO: 38 - protein, 6 aa, Artificial Sequence
  Description of Artificial Sequence Synthetic peptide
LYGAQF

SEQ ID NO: 39 - protein, 6 aa, Artificial Sequence
  Description of Artificial Sequence Synthetic peptide
LSQAQG

SEQ ID NO: 40 - protein, 6 aa, Artificial Sequence
  Description of Artificial Sequence Synthetic peptide
ASAASG

SEQ ID NO: 41 - protein, 6 aa, Artificial Sequence
  Description of Artificial Sequence Synthetic peptide
FLGASL

SEQ ID NO: 42 - protein, 6 aa, Artificial Sequence
  Description of Artificial Sequence Synthetic peptide
AYGATG

SEQ ID NO: 43 - protein, 6 aa, Artificial Sequence
  Description of Artificial Sequence Synthetic peptide
LAQATG

SEQ ID NO: 44 - protein, 8 aa, Artificial Sequence
  Description of Artificial Sequence Synthetic peptide
GPLGIAGQ

SEQ ID NO: 45 - protein, 10 aa, Artificial Sequence
  Description of Artificial Sequence Synthetic peptide
GGPLGMLSQS

SEQ ID NO: 46 - protein, 6 aa, Artificial Sequence
  Description of Artificial Sequence Synthetic peptide
PLGLAG

SEQ ID NO: 47 - protein, 6 aa, Artificial Sequence
  Description of Artificial Sequence Synthetic peptide
AANLRN

SEQ ID NO: 48 - protein, 6 aa, Artificial Sequence
  Description of Artificial Sequence Synthetic peptide
AQAYVK

SEQ ID NO: 49 - protein, 6 aa, Artificial Sequence
  Description of Artificial Sequence Synthetic peptide
AANYMR

SEQ ID NO: 50 - protein, 6 aa, Artificial Sequence
  Description of Artificial Sequence Synthetic peptide
AAALTR

SEQ ID NO: 51 - protein, 6 aa, Artificial Sequence
  Description of Artificial Sequence Synthetic peptide
AQNLMR

SEQ ID NO: 52 - protein, 6 aa, Artificial Sequence
  Description of Artificial Sequence Synthetic peptide
AANYTK

SEQ ID NO: 53 - protein, 5 aa, Artificial Sequence
  Description of Artificial Sequence Synthetic peptide
RRRRR

SEQ ID NO: 54 - protein, 6 aa, Artificial Sequence
  Description of Artificial Sequence Synthetic peptide
RRRRRR

SEQ ID NO: 55 - protein, 10 aa, Artificial Sequence
  Description of Artificial Sequence Synthetic peptide
GQSSRHRRAL

SEQ ID NO: 56 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 228 aa, Homo sapiens
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 59 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 61 - protein, 177 aa, Macaca fascicularis
QDGNEEMGSITQTPYQVSISGTTVILTCSQHLGSEAQWQHNGKNKGDSGDQLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 62 - protein, 108 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMS

SEQ ID NO: 63 - protein, 27 aa, Homo sapiens
QDGNEEMGGITQTPYKVSISGTTVILT

SEQ ID NO: 64 - protein, 6 aa, Homo sapiens
QDGNEE

SEQ ID NO: 65 - protein, 125 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 66 - protein, 109 aa, Artificial Sequence
  Description of Artificial Sequence Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 67 - protein, 125 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSS

SEQ ID NO: 68 - protein, 109 aa, Artificial Sequence
  Description of Artificial Sequence Synthetic polypeptide
QTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 69 - protein, 5 aa, Artificial Sequence
  Description of Artificial Sequence Synthetic peptide
KYAMN

SEQ ID NO: 70 - protein, 19 aa, Artificial Sequence
  Description of Artificial Sequence Synthetic peptide
RIRSKYNNYATYYADSVKD

SEQ ID NO: 71 - protein, 14 aa, Artificial Sequence
  Description of Artificial Sequence Synthetic peptide
HGNFGNSYISYWAY

SEQ ID NO: 72 - protein, 14 aa, Artificial Sequence
  Description of Artificial Sequence Synthetic peptide
GSSTGAVTSGNYPN

SEQ ID NO: 73 - protein, 7 aa, Artificial Sequence
  Description of Artificial Sequence Synthetic peptide
GTKFLAP

SEQ ID NO: 74 - protein, 9 aa, Artificial Sequence
  Description of Artificial Sequence Synthetic peptide
VLWYSNRWV

SEQ ID NO: 75 - protein, 117 aa, Artificial Sequence
  Description of Artificial Sequence Synthetic polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 76 - protein, 107 aa, Artificial Sequence
  Description of Artificial Sequence Synthetic polypeptide
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 77 - protein, 117 aa, Artificial Sequence
  Description of Artificial Sequence Synthetic polypeptide
QVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSS

SEQ ID NO: 78 - protein, 107 aa, Artificial Sequence
  Description of Artificial Sequence Synthetic polypeptide
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIK

SEQ ID NO: 79 - protein, 5 aa, Artificial Sequence
  Description of Artificial Sequence Synthetic peptide
DYYMT

SEQ ID NO: 80 - protein, 17 aa, Artificial Sequence
  Description of Artificial Sequence Synthetic peptide
YISSSGSTIYYADSVKG

SEQ ID NO: 81 - protein, 8 aa, Artificial Sequence
  Description of Artificial Sequence Synthetic peptide
DRNSHFDY

SEQ ID NO: 82 - protein, 11 aa, Artificial Sequence
  Description of Artificial Sequence Synthetic peptide
RASQGINTWLA

SEQ ID NO: 83 - protein, 7 aa, Artificial Sequence
  Description of Artificial Sequence Synthetic peptide
GASGLQS

SEQ ID NO: 84 - protein, 9 aa, Artificial Sequence
  Description of Artificial Sequence Synthetic peptide
QQAKSFPRT

SEQ ID NO: 85 - protein, 484 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 227 aa, Artificial Sequence; identical to SEQ ID NO: 86
  Description of Artificial Sequence Synthetic polypeptide

SEQ ID NO: 88 - protein, 982 aa, Artificial Sequence
  Description of Artificial Sequence Synthetic polypeptide
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 1010 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSGGGGSQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGPLGMLSQSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 1011 aa, Artificial Sequence
  Description of Artificial Sequence Synthetic polypeptide
QDGNEEGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGGPLGMLSQSGGGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIKSGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 1008 aa, Artificial Sequence
  Description of Artificial Sequence Synthetic polypeptide
QDGNEESGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSSGGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGPLGMLSQSGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIK

SEQ ID NO: 92 - protein, 1038 aa, Artificial Sequence
  Description of Artificial Sequence Synthetic polypeptide
QDGNEESGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSSGGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSGGGGSGGGGSGGGGSGGGGSGGGGSGGGGSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGPLGMLSQSGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIK

SEQ ID NO: 93 - protein, 993 aa, Artificial Sequence
  Description of Artificial Sequence Synthetic polypeptide
QDGNEESGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSSGGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGPLGMLSQSGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIK

SEQ ID NO: 94 - protein, 1628 aa, Artificial Sequence
  Description of Artificial Sequence Synthetic polypeptide
QDGNEESGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGGPLGMLSQSGGGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIKSGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 1649 aa, Artificial Sequence
  Description of Artificial Sequence Synthetic polypeptide
QDGNEEMGGITQTPYKVSISGTTVILTSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGGPLGMLSQSGGGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIKSGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 50 aa, Artificial Sequence
  Description of Artificial Sequence Synthetic polypeptide
  MISC_FEATURE (1)..(50): This sequence may encompass 2-10 “Gly Gly Gly Gly Ser” repeating units
GGGGSGGGGSGGGGSGGGGSGGGGSGGGGSGGGGSGGGGSGGGGSGGGGS

SEQ ID NO: 97 - protein, 50 aa, Artificial Sequence
  Description of Artificial Sequence Synthetic polypeptide
  MISC_FEATURE (1)..(50): This sequence may encompass 2-10 “Gly Gly Gly Gly Gln” repeating units
GGGGQGGGGQGGGGQGGGGQGGGGQGGGGQGGGGQGGGGQGGGGQGGGGQ

SEQ ID NO: 98 - protein, 50 aa, Artificial Sequence; identical to SEQ ID NO: 96
  Description of Artificial Sequence Synthetic polypeptide
  MISC_FEATURE (1)..(50): This sequence may encompass 3-10 “Gly Gly Gly Gly Ser” repeating units

SEQ ID NO: 99 - protein, 50 aa, Artificial Sequence; identical to SEQ ID NO: 97
  Description of Artificial Sequence Synthetic polypeptide
  MISC_FEATURE (1)..(50): This sequence may encompass 3-10 “Gly Gly Gly Gly Gln” repeating units

SEQ ID NO: 100 - protein, 4 aa, Artificial Sequence
  Description of Artificial Sequence Synthetic peptide
GGGG

SEQ ID NO: 101 - protein, 40 aa, Artificial Sequence
  Description of Artificial Sequence Synthetic polypeptide
  MISC_FEATURE (1)..(40): This sequence may encompass 1-10 “Gly Gly Gly Gly” repeating units
GGGGGGGGGGGGGGGGGGGGGGGGGGGGGGGGGGGGGGGG

SEQ ID NO: 102 - protein, 8 aa, Artificial Sequence
  Description of Artificial Sequence Synthetic peptide
AVRWLLTA

SEQ ID NO: 103 - protein, 6 aa, Artificial Sequence
  Description of Artificial Sequence Synthetic 6xHis tag
HHHHHH

SEQ ID NO: 104 - protein, 5 aa, Unknown
  Description of Unknown CD3-epsilon chain epitope sequence
QDGNE

SEQ ID NO: 105 - protein, 30 aa, Artificial Sequence
  Description of Artificial Sequence Synthetic polypeptide
GGGGSGGGGSGGGGSGGGGSGGGGSGGGGS

SEQ ID NO: 106 - protein, 60 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGSGGGGS

SEQ ID NO: 107 - protein, 1032 aa, Artificial Sequence
  Description of Artificial Sequence Synthetic polypeptide
QDGNEESGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGGPLGMLSQSGGGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIK

SEQ ID NO: 108 - protein, 1024 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGGPLGMLSQSGGGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIK

SEQ ID NO: 109 - protein, 1620 aa, Artificial Sequence
  Description of Artificial Sequence Synthetic polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSGGPLGMLSQSGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGPLGMLSQSGQVQLVESGGGLVKPGGSLRLSCAASGFTFSDYYMTWIRQAPGKCLEWLSYISSSGSTIYYADSVKGRFTISRDNAKNSLFLQMNSLRAEDTAVYYCARDRNSHFDYWGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLSGGGPLGMLSQSGGGDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGCGTKVEIKSGPLGMLSQSGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 40 aa, Artificial Sequence
  Description of Artificial Sequence Synthetic polypeptide
GGGGSGGGGSGGGGSGGGGSGGGGSGGGGSGGGGSGGGGS